(19) United States
(12) Patent Application Publication
(10) Pub. No.: US 2025/0305036 A1
(43) Pub. Date: Oct. 2, 2025

(54) METHODS, COMPOSITONS AND SYSTEMS FOR ANALYTE DETECTION

(71) Applicant: Stellaromics, Inc., Boston, MA (US)

(72) Inventor: Ye FU, Natick, MA (US)

(21) Appl. No.: 19/236,720

(22) Filed: Jun. 12, 2025

Related U.S. Application Data
(63) Continuation of application No. PCT/US2024/036943, filed on Jul. 5, 2024.
(60) Provisional application No. 63/512,502, filed on Jul. 7, 2023.

(51) Int. Cl. C12Q 1/6841; C12Q 1/6804; C12Q 1/6806; C12Q 1/6809; C12Q 1/682; C12Q 1/6827; G01N 33/68

(52) U.S. Cl. CPC C12Q 1/6841; C12Q 1/6809; C12Q 1/6827; G01N 33/6875; C12Q 1/6804; C12Q 1/6806; C12Q 1/682

(57) ABSTRACT

Provided herein are methods and systems for identifying a proximity of analytes in a sample. The method may comprise contacting one or more analytes with one or more probes. The proximity of the one or more analytes to each other may cause a ligation event between the one or more probes. An amplification reaction may be performed comprising one or more copies of a barcode or derivative thereof. The one or more copies of the barcode or derivative thereof may be detected to identify a proximity between the one or more analytes.

### CROSS-REFERENCE

This application is a continuation of International Application No. PCT/US2024/036943, filed Jul. 5, 2024, which claims priority to U.S. Provisional Patent Application No. 63/512,502, filed Jul. 7, 2023, which is entirely incorporated herein by reference.

### BACKGROUND

The proximity of analytes within a sample have been analyzed and used for determining the state of the sample. Methods have been developed for analyzing the proximity of analytes within a sample.

### SUMMARY

Aspects disclosed herein provide methods of detecting analytes in a sample, the method comprising: a) providing a first probe and a second probe, wherein the first probe comprises: (i) a first binding site configured to couple to a first analyte at a first portion of the first analyte; (ii) a second binding site configured to couple to the first analyte at a second portion of the first analyte, wherein the first portion of the first analyte is adjacent to the second portion of the first analyte; (iii) a third binding site configured to couple to the second probe; (iv) a barcode; (v) a first end; and (vi) a second end; wherein the second probe comprises: (i) a fourth binding site configured to couple to the first probe; and (ii) a fifth binding site configured to couple to the second analyte; b) contacting a sample comprising a plurality of analytes comprising the first analyte and the second analyte with the first probe and the second probe, such that: (i) the first probe is coupled to the first analyte; (ii) the second probe is coupled to the second analyte; and (iii) the first probe is coupled to the second probe; c) ligating the first end and the second end to form a circular oligonucleotide; d) amplifying the circular oligonucleotide to generate an amplicon, wherein the amplicon comprises a complement of the barcode; and e) detecting the complement of the barcode or a derivative thereof using a plurality of detection probes, thereby determining a proximity of the first analyte to the second analyte.

In some embodiments, the sample is a tissue sample. In some embodiments, the tissue sample is a fresh-frozen tissue sample. In some embodiments, the tissue sample is a formalin-fixed paraffin embedded tissue sample. In some embodiments, the sample is 5-250 μm thick. In some embodiments, the sample is 10-200 μm thick. In some embodiments, the sample is 25-150 μm thick.

In some embodiments, the first analyte comprises a nucleic acid. In some embodiments, the nucleic acid is a ribonucleic acid. In some embodiments, the ribonucleic acid is a messenger ribonucleic acid. In some embodiments, the ribonucleic acid is a ribosomal ribonucleic acid. In some embodiments, the nucleic acid is a deoxyribonucleic acid. In some embodiments, the first analyte comprises a polypeptide. In some embodiments, the comprises a ribosomal protein. In some embodiments, the first analyte comprises a chemical modification. In some embodiments, the second analyte comprises a nucleic acid. In some embodiments, the nucleic acid is a ribonucleic acid. In some embodiments, the ribonucleic acid is a messenger ribonucleic acid. In some embodiments, the ribonucleic acid is a ribosomal ribonucleic acid. In some embodiments, the nucleic acid is a deoxyribonucleic acid. In some embodiments, the second analyte comprises a polypeptide. In some embodiments, the polypeptide comprises a ribosomal protein.

In some embodiments, the first probe comprises a nucleic acid. In some embodiments, the nucleic acid comprises an oligonucleotide. In some embodiments, the oligonucleotide comprises one or more modifications. In some embodiments, the one or more modifications comprise a 5′ phosphate modification. In some embodiments, the one or more modification comprises an internucleotide linkage. In some embodiments, the internucleotide linkage is a phosphorothioate. In some embodiments, the internucleotide linkage is a phosphodiester. In some embodiments, the first probe recognizes a ribonucleic acid. In some embodiments, the nucleic acid comprises a single nucleotide polymorphism. In some embodiments, the first probe recognizes the single nucleotide polymorphism and wherein the ligating in c) does not occur if the first analyte does not comprise the single nucleotide polymorphism. In some embodiments, the first probe does not recognize the single nucleotide polymorphism and wherein the ligating in c) does not occur if the first analyte comprises the single nucleotide polymorphism. In some embodiments, the nucleic acid comprises a modification. In some embodiments, the modification is selected from the group consisting of N 6-methyladenosine, 5-methylcytosine, N 1-methyladenosine, N 7-methylguanosine, N 4-acetylcytosine, pseudouridine, and N1-methylpseudouridine. In some embodiments, the first probe recognizes the modification and wherein the ligating in c) does not occur if the first analyte does not comprise the modification. In some embodiments, the first probe does not recognize the modification and wherein the ligating in c) does not occur if the first analyte comprises the modification. In some embodiments, the first probe recognizes a deoxyribonucleic acid. In some embodiments, the first probe recognizes a deoxyribonucleic acid modification. In some embodiments, the deoxyribonucleic acid modification is a methyl modification. In some embodiments, the first probe comprises a first reactive chemical moiety at the first end and a second reactive chemical moiety at the second end. In some embodiments, the first reactive chemical moiety is selected from the group consisting of tetrazine, alkyne, azide, trans-cyclooctene, maleimide, N-hydroxysuccinimide ester, amine, carboxylic acid, hydroxyl, cyclopropenone, and thiol, norbornene. In some embodiments, the second reactive chemical moiety is selected from the group consisting of tetrazine, alkyne, azide, trans-cyclooctene, maleimide, N-hydroxysuccinimide ester, amine, carboxylic acid, hydroxyl, cyclopropenone, and thiol, norbornene. In some embodiments, the ligating in c) comprises a reaction between the first reactive chemical moiety and the second reactive chemical moiety.

In some embodiments, the ligating in c) comprises performing a ligation reaction with a ligase. In some embodiments, the ligase is a T4 ligase. In some embodiments, the second probe comprises a nucleic acid. In some embodiments, the nucleic acid comprises an oligonucleotide. In some embodiments, the oligonucleotide comprises one or more modifications. In some embodiments, the one or more modifications comprise a 5′ phosphate modification. In some embodiments, the one or more modification comprises an internucleotide linkage. In some embodiments, the internucleotide linkage is a phosphorothioate. In some embodiments, the internucleotide linkage is a phosphodiester. In some embodiments, the nucleic acid comprises an aptamer. In some embodiments, the second probe comprises a polypeptide. In some embodiments, the polypeptide comprises an antibody or antibody fragment. In some embodiments, the polypeptide comprises an affimer. In some embodiments, the polypeptide comprises a nanobody. In some embodiments, the second probe recognizes a ribonucleic acid. In some embodiments, the second probe recognizes a ribonucleic acid modification. In some embodiments, the ribonucleic acid modification is selected from the group consisting ofN 6-methyladenosine, 5-methylcytosine, N 1-methyladenosine, N 7-methylguanosine, N 4-acetylcytosine, pseudouridine, and N1-methylpseudouridine. In some embodiments, the second probe recognizes a deoxyribonucleic acid. In some embodiments, the probe recognizes a deoxyribonucleic acid modification. In some embodiments, the deoxyribonucleic acid modification is a methyl modification. In some embodiments, the second probe recognizes a polypeptide. In some embodiments, the polypeptide is a protein. In some embodiments, the protein is a transcription factor. In some embodiments, the protein is a ribosomal protein. In some embodiments, the protein is a histone. In some embodiments, the protein is a polymerase. In some embodiments, the protein is a helicase. In some embodiments, the protein is a restriction enzyme. In some embodiments, the protein is a ribonucleic acid binding protein. In some embodiments, the second probe recognizes a post-translational modification of the protein.

In some embodiments, the barcode comprises a nucleic acid. In some embodiments, the nucleic acid is deoxyribonucleic acid. In some embodiments, the nucleic acid is ribonucleic acid. In some embodiments, the nucleic acid is at least 4 nucleotides in length. In some embodiments, the nucleic acid is at least 6 nucleotides in length. In some embodiments, the nucleic acid is at least 8 nucleotides in length. In some embodiments, the nucleic acid is at least 10 nucleotides in length. In some embodiments, the barcode corresponds to the first analyte. In some embodiments, the barcode corresponds to the second analyte. In some embodiments, the barcode corresponds to the first analyte being proximal to the second analyte. In some embodiments, the first probe further comprises a second barcode. In some embodiments, the second barcode comprises a nucleic acid. In some embodiments, the nucleic acid is deoxyribonucleic acid. In some embodiments, the nucleic acid is ribonucleic acid. In some embodiments, the nucleic acid is at least 4 nucleotides in length. In some embodiments, the nucleic acid is at least 6 nucleotides in length. In some embodiments, the nucleic acid is at least 8 nucleotides in length. In some embodiments, the nucleic acid is at least 10 nucleotides in length. In some embodiments, the second barcode corresponds to the first analyte. In some embodiments, the second barcode corresponds to the second analyte. In some embodiments, the second barcode corresponds to the first analyte being proximal to the second analyte.

In some embodiments, the first binding site comprises a nucleic acid and wherein the nucleic acid is at least 6 nucleotides in length. In some embodiments, the first binding site comprises a nucleic acid and wherein the nucleic acid is at least 10 nucleotides in length. In some embodiments, the first binding site comprises a nucleic acid and wherein the nucleic acid is at least 14 nucleotides in length. In some embodiments, the first binding site comprises a nucleic acid and wherein the nucleic acid is at least 20 nucleotides in length. In some embodiments, the second binding site comprises a nucleic acid and wherein the nucleic acid is at least 4 nucleotides in length. In some embodiments, the second binding site comprises a nucleic acid and wherein the nucleic acid is at least 10 nucleotides in length. In some embodiments, the second binding site comprises a nucleic acid and wherein the nucleic acid is at least 14 nucleotides in length. In some embodiments, the second binding site comprises a nucleic acid and wherein the nucleic acid is at least 20 nucleotides in length. In some embodiments, the third binding site comprises a nucleic acid and wherein the nucleic acid is at least 4 nucleotides in length. In some embodiments, the third binding site comprises a nucleic acid and wherein the nucleic acid is at least 8 nucleotides in length. In some embodiments, the third binding site comprises a nucleic acid and wherein the nucleic acid is at least 10 nucleotides in length. In some embodiments, the third binding site comprises a nucleic acid and wherein the nucleic acid is at least 12 nucleotides in length. In some embodiments, the fourth binding site comprises a nucleic acid and wherein the nucleic acid is at least 4 nucleotides in length. In some embodiments, the fourth binding site comprises a nucleic acid and wherein the nucleic acid is at least 8 nucleotides in length. In some embodiments, the fourth binding site comprises a nucleic acid and wherein the nucleic acid is at least 10 nucleotides in length. In some embodiments, the fourth binding site comprises a nucleic acid and wherein the nucleic acid is at least 12 nucleotides in length. In some embodiments, the fifth binding site comprises a nucleic acid and wherein the nucleic acid is at least 6 nucleotides in length. In some embodiments, the fifth binding site comprises a nucleic acid and wherein the nucleic acid is at least 8 nucleotides in length. In some embodiments, the fifth binding site comprises a nucleic acid and wherein the nucleic acid is at least 10 nucleotides in length. In some embodiments, the fifth binding site comprises a nucleic acid and wherein the nucleic acid is at least 12 nucleotides in length.

In some embodiments, (c) involves performing rolling circle amplification. In some embodiments, (d) comprises hybridizing a detection probe and an anchor probe of the plurality of detection probes to the amplicon. In some embodiments, the detection probe and the section detection probe are ligated. In some embodiments, the detection probe comprises a label. In some embodiments, the label comprises a fluorescent molecule. In some embodiments, the label comprises a quantum dot. In some embodiments, the label comprises an enzyme. In some embodiments, the enzyme generates a signal indicative of the label. In some embodiments, (e) comprises detecting the label. In some embodiments, (e) comprises in situ sequencing using the plurality of detection probes. In some embodiments, (e) comprises imaging the sample. In some embodiments, the first probe recognizes a messenger ribonucleic acid, and the second probe recognizes a ribosomal protein. In some embodiments, the second probe comprises and antibody or antibody fragment. In some embodiments, the first probe recognizes a messenger ribonucleic acid, and the second probe recognizes a ribosomal ribonucleic acid. In some embodiments, the first probe recognizes a messenger ribonucleic acid, and the second probe recognizes a messenger ribonucleic acid modification. In some embodiments, the second probe comprises a reactive chemical moiety. In some embodiments, the second probe comprises an antibody or antibody fragment. In some embodiments, the first probe recognizes a deoxyribonucleic acid, and the second probe recognizes a deoxyribonucleic acid modification. In some embodiments, the second probe comprises a reactive chemical moiety. In some embodiments, the second probe comprises an antibody or antibody fragment. In some embodiments, the sample is embedded in a hydrogel. In some embodiments, the ligating in c) comprises ligating the first end to the second end. In some embodiments, the first end and the second end are separated by at least one nucleotide when coupled after (b). In some embodiments, the method further comprises performing a gap filling reaction after (b) and prior to (c), such that the first end and the third end are directly adjacent to each other.

Aspects disclosed herein provide methods of detecting analytes in a sample, the method comprising: a) providing a first probe, wherein the first probe comprises: (i) a first binding site configured to couple to a first analyte at a first portion of the first analyte; (ii) a second binding site configured to couple to the first analyte at a second portion, wherein the first portion of the first analyte is adjacent to the second portion of the first analyte; (iii) a third binding site configured to couple to the second probe; (iv) a barcode; (v) a first end; and (vi) a second end b) contacting a sample comprising a plurality of analytes comprising the first analyte and the second analyte with the first probe, such that the first probe couples to the first analyte, wherein the first end and the second end are separated by a gap; c) performing a gap-filling reaction to fill the gap; d) ligating the first end and the second end to form a circular oligonucleotide, e) contacting the circular oligonucleotide with a second probe, wherein the second probe comprises: (i) a fourth binding site that couples to the first probe; and (ii) a fifth binding site that couples to the second analyte; f) amplifying the circular oligonucleotide to generate an amplicon, wherein the amplicon comprises a complement of the barcode; and g) detecting the complement of the barcode or a derivative thereof using at least one detection probe, thereby determining a proximity of the first analyte to the second analyte.

Aspects disclosed herein provide methods of detecting analytes in a sample, the method comprising: a) Providing a first probe, a second probe, and a third probe, wherein the first probe comprises: (i) a first binding site configured to couple to a first analyte; (ii) a second binding site configured to couple to the second probe; (iii) a barcode; (iv) a first end; and (v) a second end; wherein the second probe comprises: (i) a third binding site configured to couple to the first probe; (ii) a fourth binding site configured to couple to the third probe; and (iii) a fifth binding site configured to couple to a second analyte; and wherein the third probe comprises: (i) a sixth binding site configured to couple to the second probe; (ii) a seventh binding site configured to couple to the first analyte; (iii) a third end, wherein the third end is adjacent to the first end; and (iv) a fourth end, wherein the fourth end is adjacent to the second end; b) contacting a sample comprising a plurality of analytes comprising the first analyte and the second analyte with the first probe, the second probe, and the third probe such that: (i) the first probe is coupled to the first analyte; (ii) the second probe is coupled to the second analyte; (iii) the third probe is coupled to the first analyte; (iv) the first probe is coupled to the second probe; and (v) the third probe is coupled to the second probe; c) ligating the first end and the third end and ligating the second end the fourth end to form a circular oligonucleotide; d) amplifying the circular oligonucleotide to generate an amplicon, wherein the amplicon comprises a complement of the barcode; and e) detecting the complement of the barcode or a derivative thereof using a plurality of detection probes, thereby determining a proximity of the first analyte to the second analyte.

In some embodiments, the sample is a tissue sample. In some embodiments, the tissue sample is a fresh-frozen tissue sample. In some embodiments, the tissue sample is a formalin-fixed paraffin embedded tissue sample. In some embodiments, the sample is 5-250 μm thick. In some embodiments, the sample is 10-200 μm thick. In some embodiments, the sample is 25-150 μm thick. In some embodiments, the first analyte comprises a nucleic acid. In some embodiments, the nucleic acid is a ribonucleic acid. In some embodiments, the messenger ribonucleic acid is a messenger ribonucleic acid. In some embodiments, the messenger ribonucleic acid is a ribosomal messenger ribonucleic acid. In some embodiments, the nucleic acid is a deoxyribonucleic acid. In some embodiments, the nucleic acid comprises a single nucleotide polymorphism. In some embodiments, the first probe recognizes the single nucleotide polymorphism and wherein the ligating in c) does not occur if the first analyte does not comprise the single nucleotide polymorphism. In some embodiments, the first probe does not recognize the single nucleotide polymorphism and wherein the ligating in c) does not occur if the first analyte comprises the single nucleotide polymorphism. In some embodiments, the third probe recognizes the single nucleotide polymorphism and wherein the ligating in c) does not occur if the first analyte does not comprise the single nucleotide polymorphism. In some embodiments, the third probe does not recognize the single nucleotide polymorphism and wherein the ligating in c) does not occur if the first analyte comprises the single nucleotide polymorphism. In some embodiments, the nucleic acid comprises a modification. In some embodiments, the modification is selected from the group consisting of N 6-methyladenosine, 5-methylcytosine, N 1-methyladenosine, N 7-methylguanosine, N 4-acetylcytosine, pseudouridine, and N1-methylpseudouridine. In some embodiments, the first probe recognizes the modification and wherein the ligating in c) does not occur if the first analyte does not comprise the modification. In some embodiments, the first probe does not recognize the modification and wherein the ligating in c) does not occur if the first analyte comprises the modification. In some embodiments, the third probe recognizes the modification and wherein the ligating in c) does not occur if the first analyte does not comprise the modification. In some embodiments, the third probe does not recognize the modification and wherein the ligating in c) does not occur if the first analyte comprises the modification. In some embodiments, the first analyte comprises a polypeptide. In some embodiments, the polypeptide comprises a ribosomal protein. In some embodiments, the first analyte comprises a chemical modification.

In some embodiments, the second analyte comprises a nucleic acid. In some embodiments, the nucleic acid is a ribonucleic acid. In some embodiments, the ribonucleic acid is a messenger ribonucleic acid. In some embodiments, the ribonucleic acid is a ribosomal ribonucleic acid. In some embodiments, the nucleic acid is a deoxyribonucleic acid. In some embodiments, the second analyte comprises a polypeptide. In some embodiments, the polypeptide comprises a ribosomal protein.

In some embodiments, the first probe comprises a nucleic acid. In some embodiments, the nucleic acid comprises an oligonucleotide. In some embodiments, the oligonucleotide comprises one or more modifications. In some embodiments, the one or more modifications comprise a 5′ phosphate modification. In some embodiments, the one or more modification comprises an internucleotide linkage. In some embodiments, the internucleotide linkage is a phosphorothioate. In some embodiments, the internucleotide linkage is a phosphodiester. In some embodiments, the first probe recognizes a ribonucleic acid. In some embodiments, the first probe recognizes a ribonucleic acid modification. In some embodiments, the ribonucleic acid modification is selected from the group consisting of N 6-methyladenosine, 5-methylcytosine, N 1-methyladenosine, N 7-methylguanosine, N 4-acetylcytosine, pseudouridine, and N1-methylpseudouridine. In some embodiments, the first probe recognizes a deoxyribonucleic acid. In some embodiments, the first probe recognizes a deoxyribonucleic acid modification. In some embodiments, the deoxyribonucleic acid modification is a methyl modification. In some embodiments, the first probe comprises a first reactive chemical moiety at the first end and the third probe comprises a second reactive chemical moiety at the third end. In some embodiments, the first reactive chemical moiety is selected from the group consisting of tetrazine, alkyne, azide, trans-cyclooctene, maleimide, N-hydroxysuccinimide ester, amine, carboxylic acid, hydroxyl, cyclopropenone, and thiol, norbornene. In some embodiments, the second reactive chemical moiety is selected from the group consisting of tetrazine, alkyne, azide, trans-cyclooctene, maleimide, N-hydroxysuccinimide ester, amine, carboxylic acid, hydroxyl, cyclopropenone, and thiol, norbornene. In some embodiments, the ligating in c) comprises a reaction between the first reactive chemical moiety and the second reactive chemical moiety. In some embodiments, the first probe comprises a third reactive chemical moiety at the second end and the third probe comprises a fourth reactive chemical moiety at the fourth end. In some embodiments, the third reactive chemical moiety is selected from the group consisting of tetrazine, alkyne, azide, trans-cyclooctene, maleimide, N-hydroxysuccinimide ester, amine, carboxylic acid, hydroxyl, cyclopropenone, and thiol, norbornene. In some embodiments, the fourth reactive chemical moiety is selected from the group consisting of tetrazine, alkyne, azide, trans-cyclooctene, maleimide, N-hydroxysuccinimide ester, amine, carboxylic acid, hydroxyl, cyclopropenone, and thiol, norbornene. In some embodiments, the ligating in c) comprises a reaction between the first reactive chemical moiety and the second reactive chemical moiety. In some embodiments, the ligating in c) comprises performing a ligation reaction with a ligase. In some embodiments, the ligase is a T4 ligase.

In some embodiments, the second probe comprises a nucleic acid. In some embodiments, the nucleic acid comprises an oligonucleotide. In some embodiments, the oligonucleotide comprises one or more modifications. In some embodiments, the one or more modifications comprise a 5′ phosphate modification. In some embodiments, the one or more modification comprises an internucleotide linkage. In some embodiments, the internucleotide linkage is a phosphorothioate. In some embodiments, the internucleotide linkage is a phosphodiester. In some embodiments, the nucleic acid comprises an aptamer. In some embodiments, the second probe comprises a polypeptide. In some embodiments, the polypeptide comprises an antibody or antibody fragment. In some embodiments, the polypeptide comprises an affimer. In some embodiments, the polypeptide comprises a nanobody. In some embodiments, the second probe recognizes a ribonucleic acid. In some embodiments, the second probe recognizes a ribonucleic acid modification. In some embodiments, the ribonucleic acid modification is selected from the group consisting of N 6-methyladenosine, 5-methylcytosine, N 1-methyladenosine, N 7-methylguanosine, N 4-acetylcytosine, pseudouridine, and N1-methylpseudouridine. In some embodiments, the second probe recognizes a deoxyribonucleic acid. In some embodiments, the second probe recognizes a deoxyribonucleic acid modification. In some embodiments, the deoxyribonucleic acid modification is a methyl modification. In some embodiments, the second probe recognizes a polypeptide. In some embodiments, the polypeptide is a protein. In some embodiments, the protein is a transcription factor. In some embodiments, the protein is a ribosomal protein. In some embodiments, the protein is a histone. In some embodiments, the protein is a polymerase. In some embodiments, the protein is a helicase. In some embodiments, the protein is a restriction enzyme. In some embodiments, the protein is a ribonucleic acid binding protein. In some embodiments, the second probe recognizes a post-translational modification of the protein. In some embodiments, the second probe comprises a reactive chemical moiety. In some embodiments, the third reactive chemical moiety is selected from the list consisting of tetrazine, alkyne, azide, trans-cyclooctene, maleimide, N-hydroxysuccinimide ester, amine, carboxylic acid, hydroxyl, cyclopropenone, and thiol, norbornene. In some embodiments, the reactive chemical moiety reacts with the second analyte. In some embodiments, the reactive chemical moiety reacts with the first analyte.

In some embodiments, the third probe comprises a nucleic acid. In some embodiments, the nucleic acid comprises an oligonucleotide. In some embodiments, the oligonucleotide comprises one or more modifications. In some embodiments, the one or more modifications comprise a 5′ phosphate modification. In some embodiments, the one or more modification comprises an internucleotide linkage. In some embodiments, the internucleotide linkage is a phosphorothioate. In some embodiments, the internucleotide linkage is a phosphodiester. In some embodiments, the third probe recognizes a ribonucleic acid. In some embodiments, the third probe recognizes a ribonucleic acid modification. In some embodiments, the ribonucleic acid modification is selected from the group consisting of N 6-methyladenosine, 5-methylcytosine, N 1-methyladenosine, N 7-methylguanosine, N 4-acetylcytosine, pseudouridine, and N1-methylpseudouridine. In some embodiments, the third probe recognizes a deoxyribonucleic acid. In some embodiments, the third probe recognizes a deoxyribonucleic acid modification. In some embodiments, the deoxyribonucleic acid modification is a methyl modification. In some embodiments, the third probe comprises a reactive chemical moiety. In some embodiments, the reactive chemical moiety reacts with the third probe.

In some embodiments, one or more barcodes comprises a nucleic acid. In some embodiments, the nucleic acid is deoxyribonucleic acid. In some embodiments, the nucleic acid is ribonucleic acid. In some embodiments, the nucleic acid is at least 4 nucleotides in length. In some embodiments, the nucleic acid is at least 6 nucleotides in length. In some embodiments, the nucleic acid is at least 8 nucleotides in length. In some embodiments, the nucleic acid is at least 10 nucleotides in length. In some embodiments, the barcode corresponds to the first analyte. In some embodiments, the barcode corresponds to the second analyte. In some embodiments, the barcode corresponds to the first analyte being proximal to the second analyte. In some embodiments, the first probe further comprises a second barcode. In some embodiments, the second barcode comprises a nucleic acid. In some embodiments, the nucleic acid is deoxyribonucleic acid. In some embodiments, the nucleic acid is ribonucleic acid. In some embodiments, the nucleic acid is at least 4 nucleotides in length. In some embodiments, the nucleic acid is at least 6 nucleotides in length. In some embodiments, the nucleic acid is at least 8 nucleotides in length. In some embodiments, the nucleic acid is at least 10 nucleotides in length. In some embodiments, the second barcode corresponds to the first analyte. In some embodiments, the second barcode corresponds to the second analyte. In some embodiments, the second barcode corresponds to the first analyte being proximal to the second analyte. In some embodiments, the third probe comprises a third barcode. In some embodiments, the third barcode comprises a nucleic acid. In some embodiments, the nucleic acid is deoxyribonucleic acid. In some embodiments, the nucleic acid is ribonucleic acid. In some embodiments, the nucleic acid is at least 4 nucleotides in length. In some embodiments, the nucleic acid is at least 6 nucleotides in length. In some embodiments, the nucleic acid is at least 8 nucleotides in length. In some embodiments, the nucleic acid is at least 10 nucleotides in length. In some embodiments, the third barcode corresponds to the first analyte. In some embodiments, the third barcode corresponds to the second analyte. In some embodiments, the third barcode corresponds to the first analyte being proximal to the second analyte. In some embodiments, the first binding site comprises a nucleic acid and wherein the nucleic acid is at least 6 nucleotides in length. In some embodiments, the first binding site comprises a nucleic acid and wherein the nucleic acid is at least 10 nucleotides in length. In some embodiments, the first binding site comprises a nucleic acid and wherein the nucleic acid is at least 14 nucleotides in length. In some embodiments, the first binding site comprises a nucleic acid and wherein the nucleic acid is at least 20 nucleotides in length. In some embodiments, the second binding site comprises a nucleic acid and wherein the nucleic acid is at least 2 nucleotides in length. In some embodiments, the second binding site comprises a nucleic acid and wherein the nucleic acid is at least 4 nucleotides in length. In some embodiments, the second binding site comprises a nucleic acid and wherein the nucleic acid is at least 8 nucleotides in length. In some embodiments, the second binding site comprises a nucleic acid and wherein the nucleic acid is at least 10 nucleotides in length. In some embodiments, the third binding site comprises a nucleic acid and wherein the nucleic acid is at least 2 nucleotides in length. In some embodiments, the third binding site comprises a nucleic acid and wherein the nucleic acid is at least 4 nucleotides in length. In some embodiments, the third binding site comprises a nucleic acid and wherein the nucleic acid is at least 8 nucleotides in length. In some embodiments, the third binding site comprises a nucleic acid and wherein the nucleic acid is at least 12 nucleotides in length. In some embodiments, the fourth binding site comprises a nucleic acid and wherein the nucleic acid is at least 2 nucleotides in length. In some embodiments, the fourth binding site comprises a nucleic acid and wherein the nucleic acid is at least 4 nucleotides in length. In some embodiments, the fourth binding site comprises a nucleic acid and wherein the nucleic acid is at least 8 nucleotides in length. In some embodiments, the fourth binding site comprises a nucleic acid and wherein the nucleic acid is at least 12 nucleotides in length. In some embodiments, the fifth binding site comprises a nucleic acid and wherein the nucleic acid is at least 6 nucleotides in length. In some embodiments, the fifth binding site comprises a nucleic acid and wherein the nucleic acid is at least 10 nucleotides in length. In some embodiments, the fifth binding site comprises a nucleic acid and wherein the nucleic acid is at least 14 nucleotides in length. In some embodiments, the fifth binding site comprises a nucleic acid and wherein the nucleic acid is at least 20 nucleotides in length. In some embodiments, the sixth binding site comprises a nucleic acid and wherein the nucleic acid is at least 2 nucleotides in length. In some embodiments, the sixth binding site comprises a nucleic acid and wherein the nucleic acid is at least 4 nucleotides in length. In some embodiments, the sixth binding site comprises a nucleic acid and wherein the nucleic acid is at least 8 nucleotides in length. In some embodiments, the sixth binding site comprises a nucleic acid and wherein the nucleic acid is at least 12 nucleotides in length. In some embodiments, the seventh binding site comprises a nucleic acid and wherein the nucleic acid is at least 4 nucleotides in length. In some embodiments, the seventh binding site comprises a nucleic acid and wherein the nucleic acid is at least 10 nucleotides in length. In some embodiments, the seventh binding site comprises a nucleic acid and wherein the nucleic acid is at least 14 nucleotides in length. In some embodiments, the seventh binding site comprises a nucleic acid and wherein the nucleic acid is at least 20 nucleotides in length.

In some embodiments, (c) involves performing rolling circle amplification. In some embodiments, (d) comprises hybridizing a detection probe and an anchor probe of the plurality of detection probes to the amplicon. In some embodiments, the detection probe and the section detection probe are ligated. In some embodiments, the detection probe comprises a label. In some embodiments, the label comprises a fluorescent molecule. In some embodiments, the label comprises a quantum dot. In some embodiments, the label comprises an enzyme. In some embodiments, the enzyme generates a signal indicative of the label. In some embodiments, (e) comprises detecting the label. In some embodiments, (e) comprises in situ sequencing using the plurality of detection probes. In some embodiments, (e) comprises imaging the sample. In some embodiments, the first probe and third probe recognize a messenger ribonucleic acid and the second probe recognizes a ribosomal ribonucleic acid. In some embodiments, the first probe and third probe recognize a messenger ribonucleic acid and the second probe recognizes a messenger ribonucleic acid modification. In some embodiments, the second probe comprises a reactive chemical moiety. In some embodiments, the second probe comprises an antibody or antibody fragment. In some embodiments, the first probe and third probe recognize a deoxyribonucleic acid and the second probe recognizes a deoxyribonucleic acid modification. In some embodiments, the second probe comprises a reactive chemical moiety. In some embodiments, the second probe comprises an antibody or antibody fragment. In some embodiments, the sample is embedded in a hydrogel. In some embodiments, the ligating in c) comprises ligating the first end to the third end. In some embodiments, the ligating in c) comprises ligating the second end to the fourth end. In some embodiments, the first end and the third end are separated by at least one nucleotide when coupled after (b). In some embodiments, the method further comprises performing a gap filling reaction after (b) and prior to (c), such that the first end and the third end are directly adjacent to each other. In some embodiments, the second end and the fourth end are separated by at least one nucleotide when coupled after (b). In some embodiments, the method further comprises performing a gap filling reaction after (b) and prior to (c), such that the second end and the fourth end are directly adjacent to each other.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1A and FIG. 1B show a two-probe system to detect mRNA translation in situ with SplintR Ligase.

FIG. 2A and FIG. 2B show a two-probe system to detect modified RNA, or RNA/protein/DNA bound to mRNA in situ with SplintR Ligase.

FIG. 3A and FIG. 3B show a three-probe system to detect mRNA translation in situ with SplintR Ligase and DNA Ligase.

FIG. 4A and FIG. 4B show a three-probe system to detect modified RNA, or RNA/protein/DNA bound to mRNA in situ with SplintR Ligase and DNA Ligase.

FIG. 5A and FIG. 5B show a three-probe system to detect mRNA translation in situ with SplintR Ligase.

FIG. 6A and FIG. 6B show a three-probe system to detect modified RNA, or RNA/protein/DNA bound to mRNA in situ with SplintR Ligase.

FIG. 7A and FIG. 7B show a two-probe system to detect DNA modification, analytes bound to DNA, or analytes proximal to DNA.

FIG. 8A and FIG. 8B show a three-probe system to detect DNA modification, analytes bound to DNA, or analytes proximal to DNA.

FIG. 9 shows an illustration of detecting amplicon signals in single cells.

FIG. 10 shows a schematic of the binding sites within a two-probe system.

FIG. 11 shows a schematic of the binding sites within a three-probe system.

FIG. 12 shows a process for detecting a proximity of a first analyte to a second analyte.

FIG. 13 shows a process for detecting a proximity of a first analyte to a second analyte.

FIG. 14 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the singular forms “a,” “an,” and “the” include plural references unless the context clearly dictates otherwise. Any reference to “or” herein is intended to encompass “and/or” unless otherwise stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, the terms “hydrogel” may refer to a network of polymer chains that are water-insoluble, sometimes found as a colloidal gel in which water is the dispersion medium. In other words, hydrogels may be a class of polymeric materials that can absorb large amounts of water without dissolving. Hydrogels can contain over 99% water and may include natural or synthetic polymers, or a combination thereof. Hydrogels may also possess a degree of flexibility very similar to natural tissue, due to their significant water content. A detailed description of suitable hydrogels may be found in published U.S. patent application 20100055733, herein specifically incorporated by reference. As used herein, the terms “hydrogel subunits” or “hydrogel precursors” may mean hydrophilic monomers, prepolymers, or polymers that can be crosslinked, or “polymerized”, to form a three-dimensional (3D) hydrogel network. Without being bound by any scientific theory, it is believed that this fixation of the biological specimen in the presence of hydrogel subunits cross slinks the components of the specimen to the hydrogel subunits, thereby securing molecular components in place, preserving the tissue architecture and cell morphology.

As used herein, a “peptide,” “oligopeptide,” or “polypeptide” may refer to two or more amino acids joined together by an amide bond (that is, a “peptide bond”). Peptides may be linear or cyclic. Peptides may be cc, J3, γ, δ, or higher, or mixed. Peptides may comprise any mixture of amino acids as defined herein, such as comprising any combination of D, L, cc, J3, γ, δ, or higher amino acids.

As used herein, a “protein” may refer to an amino acid sequence having multiple linked amino acids. A protein may be a peptide having secondary and/or tertiary structures. A histone may be one type of protein that binds DNA and regulates its activity. Histone modifications include H3K4me1, H3K4me3, H3K36me3, H3K79me2, H3K9Ac, H3K27Ac, H4K16Ac, H3K27me3, H3K9me3, Gamma H2A.X, H3S10P or analogs thereof.

As used herein, a “nucleotide” may comprise a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. The phosphate groups may be in the mono¬, di-, or tri-phosphate form. These nucleotides are natural nucleotides, but it is to be further understood that non-natural nucleotides, modified nucleotides or analogs of the aforementioned nucleotides can also be used.

As used herein, “nucleobase” may be a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N6-methyladenine, 2-aminoadenine, 7-deazaxanthine, 7-deazaguanine, 7-deaza-adenine, N4-ethanocytosine, 2,6-diaminopurine, N6-ethano-2,6-diaminopurine, 5-methylcytosine, 5-(C₃-C₆)-alkynylcytosine, 5-alkynyluracil, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and other non-naturally occurring nucleobases.

The term “nucleic acid” or “polynucleotide” may refer to a deoxyribonucleotide or ribonucleotide polymer in either single- double-stranded form, or a combination thereof, and unless otherwise limited, encompasses known analogs of natural nucleotides that hybridize to nucleic acids in manner similar to naturally occurring nucleotides, such as peptide nucleic acids (PNAs) and phosphorothiolate DNA. Unless otherwise indicated, a particular nucleic acid sequence comprises the complementary sequence thereof. Nucleotides include, but are not limited to, ATP, dATP, CTP, dCTP, GTP, dGTP, UTP, TTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, and 2-thiocytidine, as well as the alphathiotriphosphates for all of the above, and 2′-O-methyl-ribonucleotide triphosphates for all the above bases. Modified bases include, but are not limited to, 5-Br-UTP, 5-Br-dUTP, 5-F-UTP, 5-F-dUTP, 5-propynyl dCTP, and 5-propynyl-dUTP.

As used herein, the term “SEDAL” may refer to Sequencing with Error-Correction by Dynamic Annealing and Ligation (SEDAL), a method to decode DNA sequences into multi-colored fluorescence signals that can be imaged. See Wang, Xiao, et al. “Three-dimensional intact-tissue sequencing of single-cell transcriptional states.” Science 361.6400 (2018).

As used herein, the term “complementary” may refer to two oligonucleotide sequences comprising nucleotides capable of hydrogen bonding. Sequences may be complementary at one or more bases, and/or at one or more contiguous positions.

As used herein, the term “transcript,” or “RNA transcript,” may refer to the cellular output generated from RNA polymerase-catalyzed transcription of DNA. The term “mRNA transcript” may refer to an RNA transcript with further post-transcriptional processing to remove introns. An mRNA transcript can be translated into a polypeptide.

As used herein, the term “padlock” or “padlock probe” may refer to one or more oligonucleotides specific for a target nucleic acid sequence. Padlock probes can further be complementary to a secondary nucleic acid sequence bound directly or indirectly to a protein of interest, as well as additional nucleic acid sequences. Padlock probes can contain other signaling elements, including barcode nucleic acid sequences.

As used herein, the term “amplicon” may refer to amplified nucleic acids, including amplified nucleotide sequences, wherein amplification copies and/or generates replicates of the target nucleic acid. Amplicons may be generated via isothermal amplification, rolling circle amplification, or repeated steps of denaturing, annealing, and extending to multiply a target nucleic acid.

As used herein, the term “probe” may refer to an oligonucleotide sequence complementary to specific sequences of DNA or RNA. A probe may comprise multiple sub-units, each complementary to one or more specific sequences of DNA or RNA. Probe shape and conformation can be manipulated. By placing a normally continuous complementary sequence at distal ends of a probe, it is possible to that when annealing, the probe attaches in a substantially circular shape.

As used herein, “detection-moiety” may refer to an antibody or fragments of an antibody such as Fab, probe, aptamer, or chemical group that can covalently or non-covalently bind to the analytes of interest.

The terms comprising, including, containing and various forms of these terms are synonymous with each other and are meant to be equally broad. Moreover, unless explicitly stated to the contrary, examples comprising, including, or having an element or a plurality of elements having a particular property may include additional elements, whether or not the additional elements have that property.

Provided herein are methods, compositions and systems for the detecting the proximity of analytes within a sample using probes. The detection methods described herein can detect the relationship between one or more analyte within a sample with high accuracy, high specificity, high sensitivity, or a combination thereof. A variety of types of analytes can be detected using these methods. Additionally, a variety of probe types can be used to detect one or more analyte as part of the methods described herein.

The compositions, methods, and systems provided herein may be used for measuring ribosomal activity. The compositions may include probes designed to detect mRNA translation, nucleic acid modifications, nucleic acid-interacting analytes, or a combination thereof, in the presence of a probe. The probe may be conjugated to a label. The probe may function as a primer and/or ligation template.

An aspect of the disclosure provides a method for detecting analytes in a sample. The method may comprise providing a first probe and/or a second probe. The first probe may comprise one or more binding sites. The first probe may comprise a first binding site (e.g. a first binding site of the first probe), a second binding site (e.g. a second binding site of the first probe), a third binding site (e.g. a third binding site of the first probe), or any combination thereof. The first binding site may be configured to couple to a first analyte at a first portion of the first analyte. The second binding site of the first probe (e.g. the second binding site of the first probe) may be configured to couple to the first analyte at a second portion of the first analyte. The third binding site (e.g. the third binding site of the first probe) may be configured to couple to the second probe. The first probe may comprise one or more barcodes. The first portion of the first analyte may be adjacent to the second portion of the first analyte. The second probe may comprise a first binding site (e.g. a fourth binding site of the second probe) and/or a second binding site (e.g. a fifth binding site of the second probe). The first binding site of the second probe (e.g. the fourth binding site of the second probe) may be configured to couple to the first probe. The second binding site of the second probe (e.g. the fifth binding site of the second probe) may be configured to couple to the second analyte. In some cases, a sample comprising one or more analytes may be contacted with a first probe and/or a second probe. The one or more analytes may comprise the first analyte and the second analyte Upon contacting the sample with the first probe and/or second probe, the first probe may be coupled to the first analyte. Upon contacting the sample with the first probe and/or second probe, the second probe may be coupled to the second analyte. Upon contacting the sample with the first probe and/or second probe, the first probe may be coupled to the second probe. The first probe may comprise a first end (e.g. a first end of the first probe) and/or a second end (e.g. a second end of the second probe). The first end of the first probe (e.g. the first end of the first probe) may be ligated to the second end of the second probe (e.g. the second end of the second probe) to form a circular oligonucleotide. The circular oligonucleotide may be amplified to generate an amplicon. The amplicon may comprise a complement of the barcode. In some cases, the complement of the barcode or a derivative thereof may be detected using a plurality of detection probes. The detection may determine a proximity of the first analyte to the second analyte.

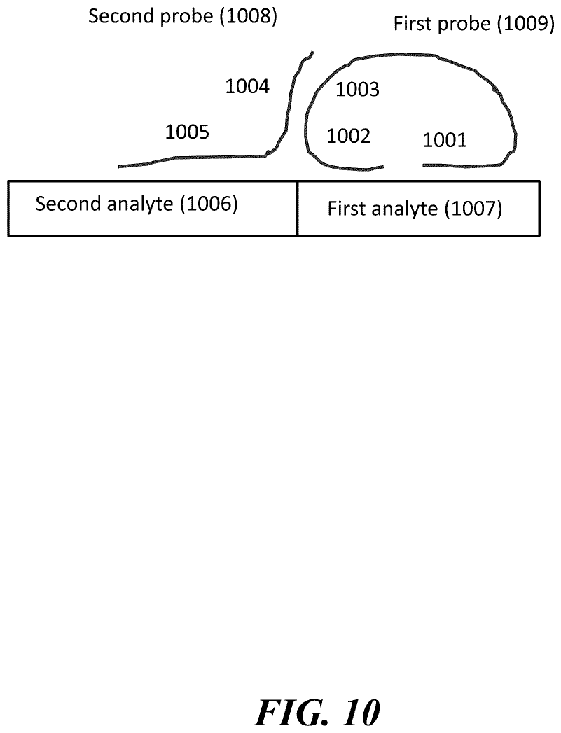

An additional aspect of the disclosure provides a method for detecting analytes in a sample using the components shown in FIG. 10. The method comprises: (a) providing a first probe (1009) and a second probe (1008), wherein the first probe (1009) comprises: (i) a first binding site (1001) configured to couple to a first analyte (1007) at a first portion of the first analyte; (ii) a second binding site (1002) configured to couple to the first analyte (1007) at a second portion of the first analyte, wherein the first portion of the first analyte is adjacent to the second portion of the first analyte; (iii) a third binding site (1003) configured to couple to the second probe (1008); (iv) a barcode; (v) a first end; and (vi) a second end; wherein the second probe (1008) comprises: (i) a fourth binding site (1004) configured to couple to the first probe (1009); and (ii) a fifth binding site (1005) configured to couple to the second analyte (1006); (b) contacting a sample comprising a plurality of analytes comprising the first analyte (1007) and the second analyte (1006) with the first probe (1009) and the second probe (1008), such that: (i) the first probe (1009) is coupled to the first analyte (1007); (ii) the second probe (1008) is coupled to the second analyte (1006); and (iii) the first probe (1009) is coupled to the second probe (1008); (c) ligating the first end and the second end to form a circular oligonucleotide; (d) amplifying the circular oligonucleotide to generate an amplicon, wherein the amplicon comprises a complement of the barcode; and (e) detecting the complement of the barcode or a derivative thereof using a plurality of detection probes, thereby determining a proximity of the first analyte (1006) to the second analyte (1007).

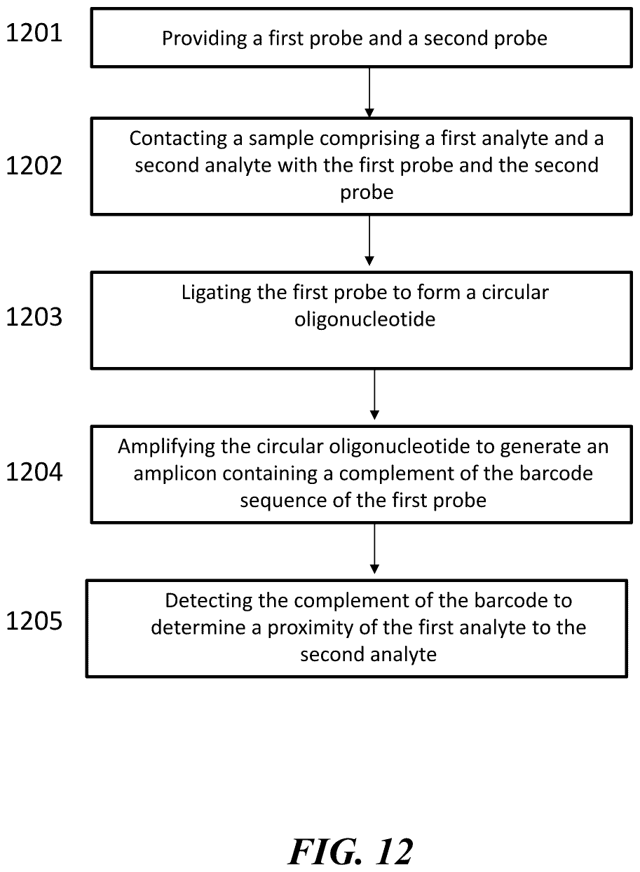

FIG. 12 schematically illustrates an example of detecting the proximity of a first analyte and a second analyte using a first probe and a second probe. In this example, a first probe and second probe are provided (1201). The first probe and second probe may comprise nucleic acid, for example. A sample comprising the first analyte and second analyte may be contacted with the first probe and the second probe (1202). The first probe may bind the first analyte and the second probe may bind the second analyte, for example. One end of the first probe may be ligated to another end of the first probe to form a circular oligonucleotide (1203). The circular oligonucleotide may be amplified to generate one or more amplicons containing a complement of a barcode sequence of the first probe (1204). The complement of the barcode sequence of the first probe may be the reverse complement of the barcode sequence, for example. The complement of the barcode may be detected to determine a proximity of the first analyte to the second analyte (1205). For example, a plurality of detection probes may be added to the sample and a subset of the plurality of the detection probes may bind to the one or more amplicons to reveal at least a portion of the complement of the barcode of the first probe.

Additional aspects of the disclosure provide a method for detecting analytes in a sample. The method may comprise providing a first probe. The first probe may comprise one or more binding sites. The first probe may comprise a first binding site, a second binding site, a third binding site, or any combination thereof. The first probe may be configured to couple to a first analyte. The first probe may comprise a first binding site configured to couple to a first analyte at a first portion of the first analyte. (ii) The second binding site may be configured to couple to the first analyte at a second portion of the first analyte. The first portion of the first analyte may be adjacent to the second portion of the first analyte. The third binding site may be configured to couple to the second probe. The first probe may comprise (iv) a barcode. The first probe may comprise a first end. The first probe may comprise a second end. In some cases, a sample comprising one or more analytes, including the first analyte and/or the second analyte may be contacted with the first probe, such that the first probe may couple to the first analyte. The first end of the first probe and the second end of the first probe may be separated by a gap (e.g. a single-stranded region of the first analyte not hybridized to the first probe and adjacent to the first end and second end of the first probe). A gap-filling reaction may be performed to fill the gap (e.g. extend the nucleic acid sequence of the first probe from one end to the other end according to the nucleic acid sequence of the single-stranded region of the first analyte not hybridized to the first probe). The first end may be ligated to the second end after the gap-filling reaction to form a circular oligonucleotide. The circular oligonucleotide may be contacted with a second probe. The second probe may comprise a fourth binding site that couples to the first probe. The second probe may comprise a fifth binding site that couples to said second analyte. The circular oligonucleotide may be amplified to generate an amplicon. The amplicon may comprise a complement of the barcode. The complement of the barcode or a derivative thereof may be detected using at least one detection probe, thereby determining a proximity of the first analyte to the second analyte.

Additional aspects disclosed herein provide methods of detecting analytes in a sample using the components shown in FIG. 10, the method comprising: a) providing a first probe (1009), wherein the first probe comprises: (i) a first binding site (1001) configured to couple to a first analyte (1007) at a first portion of the first analyte; (ii) a second binding site (1002) configured to couple to the first analyte at a second (1006), wherein the first portion of the first analyte is adjacent to the second portion of the first analyte; (iii) a third binding site (1003) configured to couple to the second probe (1008); (iv) a barcode; (v) a first end; and (vi) a second end b) contacting a sample comprising a plurality of analytes comprising the first analyte (1007) and the second analyte (1006) with the first probe (1009), such that the first probe (1009) couples to the first analyte (1007), wherein the first end and the second end are separated by a gap; c) performing a gap-filling reaction to fill the gap; d) ligating the first end and the second end to form a circular oligonucleotide, e) contacting the circular oligonucleotide with a second probe (1006), wherein the second probe comprises: (i) a fourth binding site (1004) that couples to the first probe; and (ii) a fifth binding site (1005) that couples to the second analyte (1006); f) amplifying the circular oligonucleotide to generate an amplicon, wherein the amplicon comprises a complement of the barcode; and g) detecting the complement of the barcode or a derivative thereof using at least one detection probe, thereby determining a proximity of the first analyte (1007) to the second analyte (1006).

The first probe may comprise at least one binding site configured to couple to an analyte, a probe, or a combination thereof. In some cases, the at least one binding site of the first probe may be configured to couple to an analyte. The analyte may be the first analyte. In some cases, the at least one binding site of the first probe may be configured to couple to a probe. In some cases, the probe that the first probe is configured to bind to may be the second probe. The at least one binding site of the first probe may comprise a nucleic acid. The nucleic acid of the at least one binding site of the first probe may have a length of at least about 1, at least about 2, at least about 3, at least about 4, at least about 5, at least about 6, at least about 7, at least about 8, at least about 9, at least about 10, at least about 15, at least about 20, at least about 25, at least about 30, at least about 40, at least about 50, at least about 60, at least about 70, at least about 80, at least about 90, at least about 100, or more nucleotides. The nucleic acid of the at least one binding site of the first probe may have a length of at most about 1, at most about 2, at most about 3, at most about 4, at most about 5, at most about 6, at most about 7, at most about 8, at most about 9, at most about 10, at most about 15, at most about 20, at most about 25, at most about 30, at most about 40, at most about 50, at most about 60, at most about 70, at most about 80, at most about 90, at most about 100, or fewer nucleotides. The nucleic acid may have a length of about 1 to about 100, about 2 to about 90, about 3 to about 80, about 4 to about 70, about 5 to about 60, about 6 to about 50, about 7 to about 40, about 8 to about 30, about 9 to about 25, or about 10-20 nucleotides. The nucleic acid of the at least one binding site of the first probe may comprise a variety of nucleotides, including but not limited to adenine (A), guanine (G), thymine (T), cytosine (C), uracil, or a combination thereof. In some cases, the nucleic acid may comprise a modification. The modification of the first binding site of the first probe may comprise a methylation modification, a phosphate modification, or a combination thereof. The modification may comprise a sugar modification, a sugar/backbone modification, a backbone modification, a base modification, an unnatural base pair, or a combination thereof. In some cases the sugar modification may comprise a 2′-fluoro, a 2′-O-methyl, a 2′-fluoro arabinose nucleic acid, a hexitol nucleic acid, a, 2′-O-methoxyethyl, a (1′-3′)-β-L-ribulo nucleic acid, a α-L-threose nucleic acid, a 3′-2′ phosphonomethyl-threosyl nucleic acid, a 2′-deoxyxylonucleic acid, a phosphorothioate, an alkyl phosphonate nucleic acid, a peptide nucleic acid, or a combination thereof.

The first probe may bind to an analyte at one or more portions. In some cases, the first probe may bind to one or more portion of an analyte. In some cases, the one or more portions of the analyte may be overlapping. For example, in some cases, the first probe may bind to a first portion on an analyte that comprises at least part of a second portion of the first analyte of the analyte that the first probe binds to. In cases where the first analyte comprises a nucleic acid, the first portion of the analyte and second portion of the first analyte as described herein may comprise part of the same nucleic acid sequence (e.g. part of the same nucleic acid molecule). In some cases, the one or more portions of an analyte may not be overlapping. For example, in cases where the first analyte comprises a nucleic acid, the first portion of the first analyte and second portion of the first analyte as described herein may comprise two different portions of the nucleic acid that are adjacent to each other or separated by one or more nucleotides. In some cases the one or more portions of the analyte may be separated by a distance relative to the analyte. For example, in some cases, the first analyte may comprise a nucleic acid and bind to a first portion of the first analyte and a second portion of the first analyte where the first portion of the first analyte and second portion of the first analyte are separated by at least one nucleotide. In the cases where the analyte comprises a nucleic acid, the distance between the one or more portions may be at least about 1, at least about 2, at least about 3, at least about 4, at least about 5, at least about 6, at least about 7, at least about 8, at least about 9, at least about 10, at least about 15, at least about 20, at least about 25, at least about 30, at least about 40, at least about 50, at least about 60, at least about 70, at least about 80, at least about 90, at least about 100 nucleotides. In cases where the analyte comprises a nucleic acid, the distance between the one or more portion may be at most about 1, at most about 2, at most about 3, at most about 4, at most about 5, at most about 6, at most about 7, at most about 8, at most about 9, at most about 10, at most about 15, at most about 20, at most about 25, at most about 30, at most about 40, at most about 50, at most about 60, at most about 70, at most about 80, at most about 90, at most about 100 nucleotides. In cases where the analyte comprises a nucleic acid, the distance between the one or more portion may be about 1 to about 100, about 2 to about 90, about 3 to about 80, about 4 to about 70, about 5 to about 60, about 6 to about 50, about 7 to about 40, about 8 to about 30, about 9 to about 25, or about 10 to about 20 nucleotides. In some cases, the first analyte may comprise a polypeptide. In cases where the first analyte comprises a polypeptide, the distance between the one or more portion may be at least about 1, at least about 2, at least about 3, at least about 4, at least about 5, at least about 6, at least about 7, at least about 8, at least about 9, at least about 10, at least about 15, at least about 20, at least about 25, at least about 30, at least about 40, at least about 50, at least about 60, at least about 70, at least about 80, at least about 90, at least about 100, about 1-100, about 2-90, about 3-80, about 4-70, about 5-60, about 6-50, about 7-40, about 8-30, about 9-25, or about 10-20 amino acids. In cases where the first analyte comprises a polypeptide, the distance between the one or more portion may be at most about 1, at most about 2, at most about 3, at most about 4, at most about 5, at most about 6, at most about 7, at most about 8, at most about 9, at most about 10, at most about 15, at most about 20, at most about 25, at most about 30, at most about 40, at most about 50, at most about 60, at most about 70, at most about 80, at most about 90, at most about 100, about 1 to about 100, about 2 to about 90, about 3 to about 80, about 4 to about 70, about 5 to about 60, about 6 to about 50, about 7 to about 40, about 8 to about 30, about 9 to about 25, or about 10 to about 20 amino acids.

The first probe may comprise a first binding site. The first binding site may be configured to bind to the first analyte. The first binding site may comprise a nucleic acid. The nucleic acid of the first binding site may be at least about 2, at least about 4, at least about 6, at least about 8, at least about 10, at least about 12, at least about 14, at least about 16, at least about 18, at least about 20, at least about 22, at least about 24, at least about 26, at least about 28, at least about 30, at least about 32, at least about 34, at least about 36, at least about 38, at least about 40, at least about 42, at least about 44, at least about 46, at least about 48, at least about 50, or more nucleotides in length. The nucleic acid of the first binding site may be at most about 2, at most about 4, at most about 6, at most about 8, at most about 10, at most about 12, at most about 14, at most about 16, at most about 18, at most about 20, at most about 22, at most about 24, at most about 26, at most about 28, at most about 30, at most about 32, at most about 34, at most about 36, at most about 38, at most about 40, at most about 42, at most about 44, at most about 46, at most about 48, at most about 50, or less nucleotides in length. The nucleic acid of the first binding site may be about 2-50, about 4 to about 48, about 6 to about 46, about 8 to about 44, about 10 to about 42, about 12 to about 40, about 14 to about 38, about 16 to about 36, about 18 to about 34, about 20 to about 32, about 22 to about 30, or about 24 to about 28 nucleotides in length.

The first probe may comprise a binding site (e.g. a third binding site of the first probe). The first binding site may be configured to bind to the first analyte. In some cases, the first analyte may comprise a nucleic acid. In some cases, the binding site (e.g. a third binding site of the third probe) may comprise a nucleic acid and the nucleic acid of the binding site (e.g. a third binding site of the first probe) may hybridize to at least a portion of the nucleic acid of the first analyte. The nucleic acid of the binding site (e.g. a third binding site of the first probe) may be at least about 2, at least about 4, at least about 6, at least about 8, at least about 10, at least about 12, at least about 14, at least about 16, at least about 18, at least about 20, at least about 22, at least about 24, at least about 26, at least about 28, at least about 30, at least about 32, at least about 34, at least about 36, at least about 38, at least about 40, at least about 42, at least about 44, at least about 46, at least about 48, at least about 50, or more nucleotides in length. The nucleic acid of the binding site (e.g. a third binding site of the first probe) may be at most about 2, at most about 4, at most about 6, at most about 8, at most about 10, at most about 12, at most about 14, at most about 16, at most about 18, at most about 20, at most about 22, at most about 24, at most about 26, at most about 28, at most about 30, at most about 32, at most about 34, at most about 36, at most about 38, at most about 40, at most about 42, at most about 44, at most about 46, at most about 48, at most about 50, or less nucleotides in length. The nucleic acid of the binding site (e.g. a third binding site of the first probe) may be about 2 to about 50, about 4 to about 48, about 6 to about 46, about 8 to about 44, about 10 to about 42, about 12 to about 40, about 14 to about 38, about 16 to about 36, about 18 to about 34, about 20 to about 32, about 22 to about 30, or about 24 to about 28 nucleotides in length.

The first probe may comprise a third binding site. The third binding site may be configured to bind to the second probe. For example, the third binding site may comprise a nucleic acid and the second probe may comprise a nucleic acid, and the nucleic acid of the third binding site may hybridize to the nucleic acid of the second probe. The nucleic acid of the third binding site of the first probe may be at least about 2, at least about 4, at least about 6, at least about 8, at least about 10, at least about 12, at least about 14, at least about 16, at least about 18, at least about 20, at least about 22, at least about 24, at least about 26, at least about 28, at least about 30, at least about 32, at least about 34, at least about 36, at least about 38, at least about 40, at least about 42, at least about 44, at least about 46, at least about 48, at least about 50, or more nucleotides in length. The nucleic acid of the third binding site of the first probe may be at most about 2, at most about 4, at most about 6, at most about 8, at most about 10, at most about 12, at most about 14, at most about 16, at most about 18, at most about 20, at most about 22, at most about 24, at most about 26, at most about 28, at most about 30, at most about 32, at most about 34, at most about 36, at most about 38, at most about 40, at most about 42, at most about 44, at most about 46, at most about 48, at most about 50, or less nucleotides in length. The nucleic acid of the third binding site of the first probe may be about 2 to about 50, about 4 to about 48, about 6 to about 46, about 8 to about 44, about 10 to about 42, about 12 to about 40, about 14 to about 38, about 16 to about 36, about 18 to about 34, about 20 to about 32, about 22 to about 30, or about 24 to about 28 nucleotides in length.

The first probe may ligate to itself. For example, one end of the first probe may be connected to (e.g. ligated to) another end of the first probe. The first probe may comprise a nucleic acid. The nucleic acid of the first probe may comprise one or more single-stranded regions, one or more double-stranded regions, or a combination thereof. The nucleic acid of the first probe may comprise at least one modification to enable a ligation event. In some cases, the ligation event may comprise a ligation event that connects one end of the first probe to another end of the first probe. The ligation event may comprise a chemical reaction to form a covalent bond. The ligation event may comprise the formation of a noncovalent interaction between a first end and a second end of the first probe. The first end of the first probe may be ligated to the second end of the first probe.

The chemical reaction that forms a covalent bond between one end of the first probe to another end of the first probe may be facilitated by a protein. In some cases, the protein may be an enzyme. The enzyme may be a ligase, a polymerase, a transposase, or a combination thereof. In some cases, the enzyme may be a ligase. In cases where the enzyme that facilitates the chemical reaction that forms a covalent bond between one end of the first probe to another end of the first probe is a ligase, the ligase may be one or more ligases. In some cases, the ligase may comprise a mammalian ligase. In some cases, the ligase may be a bacterial ligase. The ligase may be a DNA ligase I, DNA ligase II, DNA ligase III, DNA ligase IV, or a combination thereof. In some cases, the ligase may comprise an RNA ligase. In some cases, the ligase may ligate a 3′nucleotide of one nucleic acid to a 5′ nucleotide of a different nucleic acid. In some cases, the ligase may ligate a 3′ end of a nucleic acid to a 5′ end of the same nucleic acid. For example, a nucleic acid molecule may comprise a 5′ end and a 3′ end and the 5′ and the 3′ of the nucleic acid molecule may be ligated to form a circular oligonucleotide. The ligase may ligate two nucleotides that are part of a double-stranded nucleic acid. In some cases, the double-stranded nucleic acid may comprise a nick, and the portion of the nick may be ligated by the ligase. In some embodiments, the double-stranded nucleic acid may comprise a DNA/DNA duplex. In some embodiments, the double-stranded nucleic acid may comprise an RNA/DNA duplex. The ligase may comprise one or more of the following: T4 DNA ligase, SplintR ligase, T3 DNA ligase, T7 DNA ligase, E. coli DNA ligase, Taq ligase, RtcB ligase, or a combination thereof.

The chemical reaction to form a covalent bond between one end of a probe and another end of a probe (e.g. could be two different ends of the same probe) may comprise a reaction between one or more chemical reactive moieties. The one or more chemical reactive moieties may comprise a chemoselective reagent. The one or more chemical reactive moieties may comprise of a tetrazine, an alkyne, an azide, a trans-cyclooctene, a maleimide, an N-hydroxysuccinimide ester, an amine, a carboxylic acid, a hydroxyl, a cyclopropenone, a thiol, a norbornene, or a combination thereof. The firs probe may comprise one or more chemical reactive moieties. The first probe may comprise a first reactive chemical moiety at the first end and a second reactive chemical moiety at the second end. The first chemical reactive moiety may comprise a tetrazine, an alkyne, an azide, a trans-cyclooctene, a maleimide, an N-hydroxysuccinimide ester, an amine, a carboxylic acid, a hydroxyl, a cyclopropenone, a thiol, a norbornene, or a combination thereof. The second chemical reactive moiety may comprise a tetrazine, an alkyne, an azide, a trans-cyclooctene, a maleimide, an N-hydroxysuccinimide ester, an amine, a carboxylic acid, a hydroxyl, a cyclopropenone, a thiol, a norbornene, or a combination thereof. The ligating step may comprise a reaction between the first chemical reactive moiety and the second chemical reactive moiety.

The first end and the second end of the first probe may be directly adjacent to each other when the first probe is coupled to the sample (e.g. the first end and second end of the first probe may be ligated without any gap filling reaction). For example, in cases where the sample comprises a nucleic acid analyte that binds to the first probe, the first end of the first probe may hybridize to a portion of the nucleic acid analyte and the second end of the first probe may hybridize to another portion of the nucleic acid analyte and there may be no gaps (i.e. singled stranded region on the nucleic acid analyte) between the 5′ of the first end and 3′ of the second end, or no gaps between the 3′ end of the first end and the 5′ end of the second end. In some cases, the first end and the second end may be separated by one or more nucleotides. For example, in cases where the sample comprises a nucleic acid analyte that binds to the first probe, the first end of the first probe may hybridize to a portion of the nucleic acid analyte and the second end of the first probe may hybridize to another portion of the nucleic acid analyte and there may be a gap (i.e. a singled stranded region on the nucleic acid analyte) between the 5′ of the first end and 3′ of the second end, or a gap between the 3′ end of the first end and the 5′ end of the second end. A gap-filling reaction may be performed to enable the first end and second end of a probe to be directly adjacent to one another (e.g. the 5′ end of the first end is directly ligatable to the 3′ end of the second end, or the 3′ end of the first end is directly ligatable to the 5′ end of the second end using a ligase). The gap-filling reaction may comprise using a polymerase and/or transcriptase to extend one or two ends of the first probe by incorporation of nucleotides according to the sequence of a nucleic acid bound by the first probe.

The first probe may ligate to itself to form a circular probe. The circular probe may be a circular nucleic acid. The circular nucleic acid may be single-stranded, double-stranded, or a combination thereof. The circular nucleic acid may bind to an analyte. In some cases, the analyte that the circular nucleic acid binds to may be the first analyte. The circular nucleic acid may bind to a probe. In some cases, the circular nucleic acid may bind to the second probe. The circular nucleic acid may comprise at least one barcode. In cases where the circular nucleic acid comprises one or more barcodes. Each of the one or more barcodes may be the same or different. For example, each of the barcodes may comprise a nucleic acid and the nucleic acid may comprise the same sequence. In other cases, each of the barcodes may comprise a nucleic acid and one or more nucleotide of the nucleic acid of each barcode may differ across the one or more barcodes.

The second probe may comprise at least one binding site configured to couple to an analyte, a probe, or a combination thereof. In some cases, the at least one binding site of the second probe may be configured to couple to one or more portions of an analyte. The one or more portions of the analyte may comprise the second analyte. In some cases, the at least one binding site of the second probe may be configured to couple to one or more portions of a probe. In some cases, the one or more portions of the probe may one or more portions of the first probe. For example, a binding site of a second probe can be configured to couple to a portion of the analyte. The binding site of the second probe may comprise a nucleic acid. The at least one binding site of the second probe may comprise a nucleic acid sequence. The nucleic acid sequence of the at least one binding site of the second probe may have a length of at least about 1, at least about 2, at least about 3, at least about 4, at least about 5, at least about 6, at least about 7, at least about 8, at least about 9, at least about 10, at least about 15, at least about 20, at least about 25, at least about 30, at least about 40, at least about 50, at least about 60, at least about 70, at least about 80, at least about 90, at least about 100, or more nucleotides. The nucleic acid of the at least one binding site of the second probe may have a length of at most about 1, at most about 2, at most about 3, at most about 4, at most about 5, at most about 6, at most about 7, at most about 8, at most about 9, at most about 10, at most about 15, at most about 20, at most about 25, at most about 30, at most about 40, at most about 50, at most about 60, at most about 70, at most about 80, at most about 90, at most about 100, or less nucleotides. The nucleic acid may have a length of about 1 to about 100, about 2 to about 90, about 3 to about 80, about 4 to about 70, about 5 to about 60, about 6 to about 50, about 7 to about 40, about 8 to about 30, about 9 to about 25, or about 10 to about 20 nucleotides. The nucleic acid of the at least one binding site of the second probe may comprise a variety of nucleotides, including but not limited to A, G, C, T, U, or a combination thereof. In some cases, the nucleic acid may comprise a modification. The modification may comprise a methylation modification, a phosphate modification, or a combination thereof. The modification may comprise a sugar modification, a sugar/backbone modification, a backbone modification, a base modification, an unnatural base pair, or a combination thereof. In some cases the sugar modification may comprise a 2′-fluoro, a 2′-O-methyl, a 2′-fluoro arabinose nucleic acid, a hexitol nucleic acid, a, 2′-O-methoxyethyl, a (1′-3′)-β-L-ribulo nucleic acid, a α-L-threose nucleic acid, a 3′-2′ phosphonomethyl-threosyl nucleic acid, a 2′-deoxyxylonucleic acid, a phosphorothioate, an alkyl phosphonate nucleic acid, a peptide nucleic acid, or a combination thereof.

The second probe may comprise a binding site (e.g. a fourth binding site of the second probe of the second probe). The binding site (e.g. a fourth binding site of the second probe) may be configured to bind to the first probe. The binding site (e.g. a fourth binding site of the second probe) of the second probe may comprise a nucleic acid sequence. The nucleic acid sequence of the binding site (e.g. a fourth binding site of the second probe) of the second probe may be at least about 2, at least about 4, at least about 6, at least about 8, at least about 10, at least about 12, at least about 14, at least about 16, at least about 18, at least about 20, at least about 22, at least about 24, at least about 26, at least about 28, at least about 30, at least about 32, at least about 34, at least about 36, at least about 38, at least about 40, at least about 42, at least about 44, at least about 46, at least about 48, at least about 50, or more nucleotides in length. The nucleic acid of the binding site (e.g. a fourth binding site of the second probe) of the second probe may be at most about 2, at most about 4, at most about 6, at most about 8, at most about 10, at most about 12, at most about 14, at most about 16, at most about 18, at most about 20, at most about 22, at most about 24, at most about 26, at most about 28, at most about 30, at most about 32, at most about 34, at most about 36, at most about 38, at most about 40, at most about 42, at most about 44, at most about 46, at most about 48, at most about 50, or less nucleotides in length. The nucleic acid of the binding site (e.g. a fourth binding site of the second probe) of the second probe may be about 2 to about 50, about 4 to about 48, about 6 to about 46, about 8 to about 44, about 10 to about 42, about 12 to about 40, about 14 to about 38, about 16 to about 36, about 18 to about 34, about 20 to about 32, about 22 to about 30, or about 24 to about 28 nucleotides in length

The second probe may comprise a binding site (e.g. a fifth binding site of the second probe). The binding site (e.g. a fifth binding site of the second probe) of the second probe may be configured to bind to the second analyte. The binding site (e.g. a fifth binding site of the second probe) may comprise a nucleic acid. The nucleic acid of the binding site (e.g. a fifth binding site of the second probe) of the second probe may be at least about 2, at least about 4, at least about 6, at least about 8, at least about 10, at least about 12, at least about 14, at least about 16, at least about 18, at least about 20, at least about 22, at least about 24, at least about 26, at least about 28, at least about 30, at least about 32, at least about 34, at least about 36, at least about 38, at least about 40, at least about 42, at least about 44, at least about 46, at least about 48, at least about 50, or more nucleotides in length. The nucleic acid of the binding site (e.g. a fifth binding site of the second probe) of the second probe may be at most about 2, at most about 4, at most about 6, at most about 8, at most about 10, at most about 12, at most about 14, at most about 16, at most about 18, at most about 20, at most about 22, at most about 24, at most about 26, at most about 28, at most about 30, at most about 32, at most about 34, at most about 36, at most about 38, at most about 40, at most about 42, at most about 44, at most about 46, at most about 48, at most about 50, or less nucleotides in length. The nucleic acid of the binding site (e.g. a fifth binding site of the second probe) of the second probe may be about 2 to about 50, about 4 to about 48, about 6 to about 46, about 8 to about 44, about 10 to about 42, about 12 to about 40, about 14 to about 38, about 16 to about 36, about 18 to about 34, about 20 to about 32, about 22 to about 30, or about 24 to about 28 nucleotides in length.

In some embodiments, the methods described herein may comprise a gap filing reaction. The gap filling reaction may be facilitated by a binding event between the first probe and the first analyte, where two ends of the first probe are separated by a distance when bound to the first analyte. For example, the first probe and first analyte may both comprise a nucleic acid, and the first probe may hybridize to the first analyte at two portions such that two ends of the first probe form double-stranded regions upon hybridizing to the first analyte. The double-stranded regions may be separated by a single-stranded region. The single-stranded region may comprise a portion of the first analyte that is not hybridized to the first probe. The single-stranded region of the first analyte may be considered a gap, and the gap-filling reaction may extend one or both ends of the first probe by adding nucleotides to the nucleic acid of the first probe according to the sequence of the first analyte not bound by the first probe. In the gap filling reactions, the first analyte may serve as a template for extending and filling in the first probe. The first probe may comprise a nucleic acid sequence and the gap may be a nucleic acid sequence comprising a single-stranded portion of the first analyte not bound by the first probe and in between a first and second end of the first probe. In some cases, the gap may comprise at least one nucleotide of the first analyte. In some cases, the gap between one end of the first probe and another end of the first probe when hybridized to the first analyte may be one or more nucleotides. In some cases the gap distance between one end of the first probe and another end of the first probe when bound to the first analyte may be at least about 1, at least about 2, at least about 3, at least about 4, at least about 5, at least about 6, at least about 7, at least about 8, at least about 9, at least about 10, at least about 15, at least about 20, at least about 25, at least about 30, at least about 40, at least about 50, at least about 60, at least about 70, at least about 80, at least about 90, at least about 100, or more nucleotides. In some cases the gap distance between one end of the first probe and another end of the first probe when bound to the first analyte may be at most about 1, at most about 2, at most about 3, at most about 4, at most about 5, at most about 6, at most about 7, at most about 8, at most about 9, at most about 10, at most about 15, at most about 20, at most about 25, at most about 30, at most about 40, at most about 50, at most about 60, at most about 70, at most about 80, at most about 90, at most about 100, or more nucleotides. In some cases, the gap distance between one end of the first probe and another end of the first probe when bound to the first analyte may be about 1 to about 100, about 2 to about 90, about 3 to about 80, about 4 to about 70, about 5 to about 60, about 6 to about 50, about 7 to about 40, about 8 to about 30, about 9 to about 25, or about 10 to about 20 nucleotides. The gap filling reaction may involve using an enzyme to connect the two ends of the first probe. The enzyme may comprise a DNA polymerase, an RNA polymerase, or a combination thereof. The DNA polymerase may comprise Q5 High-Fidelity DNA Polymerase, Q5U Hot Start High-Fidelity DNA Polymerase, Phusion High-Fidelity DNA Polymerase*, Routine PCR, OneTaq DNA Polymerase, Taq DNA Polymerase, LongAmp Taq DNA Polymerase, Hemo KlenTaq, Epimark Hot Start Taq DNA Polymerase, Isothermal Amplification and Strand Displacement, Bst DNA Polymerase, Bst DNA Polymerase, Bst 2.0 DNA Polymerase, Bst 3.0 DNA Polymerase, Bsu DNA Polymerase, Large Fragment, phi29 DNA Polymerase, phi29-XT DNA Polymerase, T7 DNA Polymerase (unmodified), Sulfolobus DNA Polymerase IV, Therminator™ DNA Polymerase, DNA Polymerase I (E. coli), DNA Polymerase I, Large (Klenow) Fragment′, Klenow Fragment (3′→5′ exo-), T4 DNA Polymerase, Legacy Polymerases, Vent DNA Polymerase, Vent (exo-) DNA Polymerase, Deep Vent DNA Polymerase, Deep Vent (exo-) DNA Polymerase, or a combination thereof.

Another aspect of the disclosure provides a method for detection analytes in a sample. The method may comprises providing a first probe, a second probe, a third probe, or any combination thereof. The first probe may comprise a first binding site (e.g. a first binding site of the first probe) configured to couple to a first analyte. The first probe may comprise a second binding site (e.g. a second binding site of the first probe) configured to couple to the second probe. The first probe may comprise one or more barcodes. The first probe may comprise a first end (e.g. a first end of the first probe). The first probe may comprise a second end (e.g. a second end of the second probe). The second probe may comprise a third binding site (e.g. a third binding site of the first probe) configured to couple to the first probe. The second probe may comprise a first binding site (e.g. a fourth binding of the second probe) configured to couple to the third probe. The second probe may comprise a second binding site (e.g. a fifth binding site of the second probe) configured to couple to a second analyte. The third probe may comprise a first binding site (e.g. a sixth binding site of the third probe) configured to couple to the second probe. The third probe may comprise a second binding site (e.g. a seventh binding site of the third probe) configured to couple to the first analyte. The third probe may comprise a first end (e.g. a third end of the third probe), wherein the first end of the third probe (e.g. the third end of the third probe) may be adjacent to the first end of the first probe (e.g. the first end of the first probe). The third probe may comprise a second end (e.g. a fourth end of the third end). The second end of the third probe (e.g. the fourth end of the third probe) may be adjacent to the second end of the first probe (e.g. the second end of the second probe). The sample comprising a plurality of analytes, comprising the first analyte and/or second analyte, may be contacted with the first probe, the second probe, and/or the third probe such that: (i) the first probe may be coupled to the first analyte; (ii) the second probe may be coupled to the second analyte; (iii) the third probe may be coupled to the first analyte; (iv) the first probe may be coupled to the second probe; and (v) the third probe may be coupled to the second probe. The first end of the first probe (e.g. the first end of the first probe) may be ligated to the first end of the third probe (e.g. the third end of the third probe) and the second end of the second probe (e.g. the second end of the first probe) may be ligated to the second end of the third probe (e.g. the fourth end of the third probe) to form a circular oligonucleotide. The circular oligonucleotide may be amplified to generate one or more amplicons. The one or more amplicons may comprise a complement of the barcode. The complement of the barcode or derivative thereof may be detected using a plurality of detection probes, thereby determining a proximity of the first analyte to the second analyte.

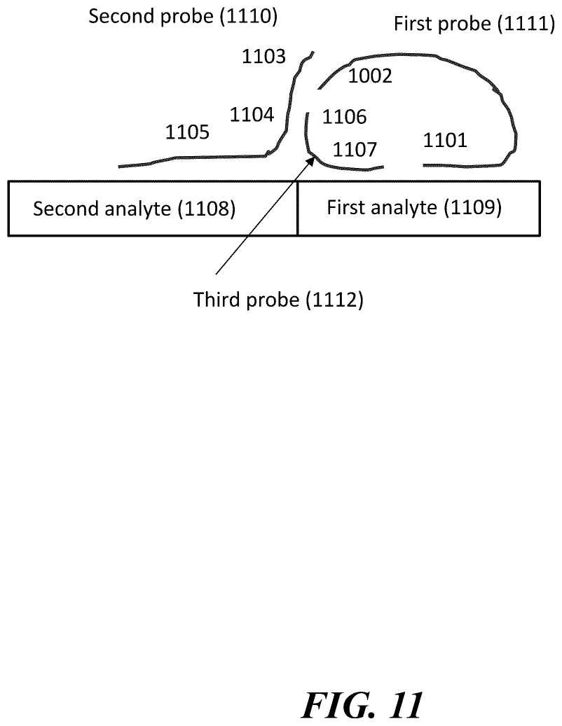

Another aspect of the disclosure provides a method for detection analytes in a sample using the components in FIG. 11. The method comprises: (a) providing a first probe (1111), a second probe (1110), and a third probe (1112), wherein the first probe (1111) comprises: (i) a first binding site (1101) configured to couple to a first analyte (1109); (ii) a second binding site (1102) configured to couple to the second probe (1110); (iii) a barcode; (iv) a first end; and (v) a second end; wherein the second probe (1110) comprises: (i) a third binding site (1103) configured to couple to the first probe (1111); (ii) a fourth binding site (1104) configured to couple to the third probe (1112); and (iii) a fifth binding site (1105) configured to couple to a second analyte (1108); and wherein the third probe (1112) comprises: (i) a sixth binding site (1106) configured to couple to the second probe (1110); (ii) a seventh binding site (1107) configured to couple to the first analyte (1109); (iii) a third end, wherein the third end is adjacent to the first end; and (iv) a fourth end, wherein the fourth end is adjacent to the second end; (b) contacting a sample comprising a plurality of analytes comprising the first analyte (1109) and the second analyte (1108) with the first probe (1111), the second probe (1110), and the third probe (1112) such that: (i) the first probe (1111) is coupled to the first analyte (1109); (ii) the second probe (1110) is coupled to the second analyte (1108); (iii) the third probe (1112) is coupled to the first analyte (1109); (iv) the first probe (1111) is coupled to the second probe (1110); and (v) the third probe (1112) is coupled to the second probe (1110); (c) ligating the first end and the third end and ligating the second end the fourth end to form a circular oligonucleotide; (d) amplifying the circular oligonucleotide to generate an amplicon, wherein the amplicon comprises a complement of the barcode; and € detecting the complement of the barcode or a derivative thereof using a plurality of detection probes, thereby determining a proximity of the first analyte (1109) to the second analyte (1108).

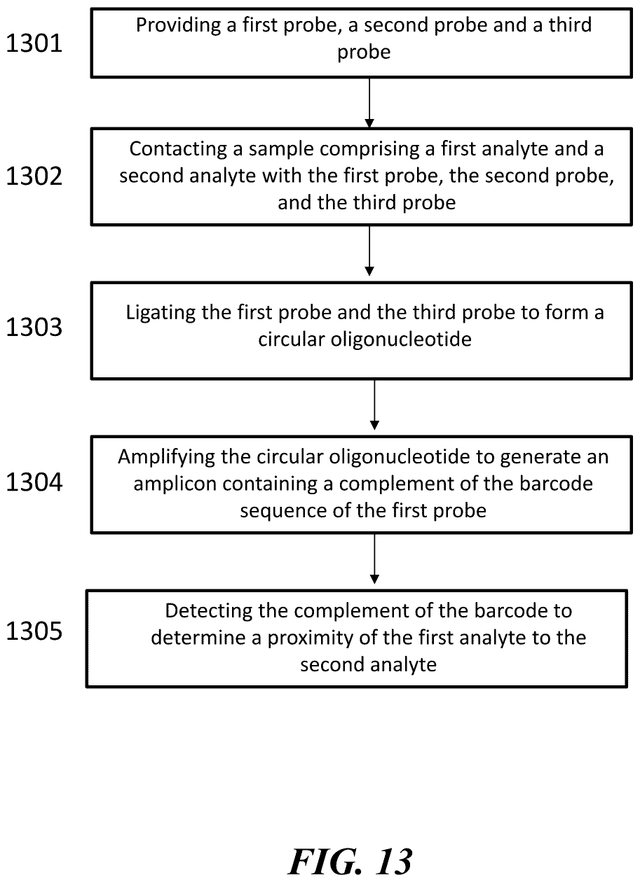

FIG. 13 schematically illustrates an example of detecting the proximity of a first analyte and a second analyte using a first probe, a second probe, and a third probe. In this example, a first probe, a second probe, and a third probe are provided (1301). The first probe, second probe, and third probe may comprise nucleic acid, for example. A sample comprising the first analyte and second analyte may be contacted with the first probe, the second probe, and the third probe (1302). The first probe may bind the first analyte, the second probe may bind the second analyte, and the third probe may bind the first analyte for example. One end of the first probe may be ligated to one end of the third probe and another end of the first probe may be ligated to another end of the third probe to form a circular oligonucleotide (1303). The circular oligonucleotide may be amplified to generate one or more amplicons containing a complement of a barcode sequence of the first probe (1304). The complement of the barcode sequence of the first probe may be the reverse complement of the barcode sequence, for example. The complement of the barcode may be detected to determine a proximity of the first analyte to the second analyte (1305). For example, a plurality of detection probes may be added to the sample and a subset of the plurality of the detection probes may bind to the one or more amplicons to reveal at least a portion of the complement of the barcode of the first probe.

In some cases, the first probe may comprise a variety of binding sites configured for a variety of purposes. The first probe may comprise at least one binding site configured to bind to at least a portion of at least one analyte, at least one probe, or a combination thereof. For example, the first probe may comprise a binding site configured to bind to the first analyte. The first binding site of the first probe may be adjacent to a binding site within the third probe. The first probe may comprise one or more binding sites configured to bind to one or more portions of the first analyte. The first probe may comprise a binding site configured to bind to one or more portions of the second probe. The first binding site of the first probe may be adjacent to a binding site within the third probe. The first probe may comprise one or more binding site configured to bind to the second probe.

The first probe may comprise a barcode. The barcode may comprise a nucleic acid. The nucleic acid of the barcode may comprise a combination of nucleotides including but not limited to A, C, G, T, U, or a combination thereof. The barcode may provide information related to the first analyte, the second analyte, or a combination thereof. For example, the information related to the first analyte, the second analyte, or a combination thereof may be sequence information, location information, expression level information, proximity information, or a combination thereof. The barcode may denote the proximity of the first analyte to the second analyte. For example, detection of the barcode may indicate that the first analyte is within 100-1000 nm of the second analyte. The barcode may be adjacent to the first binding site, the second binding site, or a combination thereof. For example, the barcode of the first probe may comprise nucleic acid and the sequence of the nucleic acid may be within 0-25 nucleotides of the first binding site of the first probe. The first binding site may comprise the barcode or a portion thereof. For example, the barcode of the first probe may comprise a nucleic acid sequence and the first binding site of the first probe may comprise another nucleic acid sequence. A portion of the nucleic acid sequence of the barcode and the nucleic acid sequence of the first binding site may be the same. For example, the nucleic acid sequence of the first binding site of the first probe may also be the barcode sequence. The second binding site may comprise the barcode or a portion thereof. For example, the barcode of the first probe may comprise a nucleic acid sequence and the second binding site of the first probe may comprise another nucleic acid sequence. A portion of the nucleic acid sequence of the barcode and the nucleic acid sequence of the second binding site may be the same. For example, the nucleic acid sequence of the second binding site of the first probe may also be the barcode sequence

The first probe may comprise one or more modifications. The one or more modifications of the first probe may be configured to ligate the first probe to the third probe. For example, one or more modification may be at an end of the first probe and one or more modifications may be at an end of the third probe. The one or more modifications of the first probe and the one or more modifications of the third probe may react with each other. In some cases, the first probe may comprise a nucleic acid, and the one or more modifications may comprise one or more nucleic acid modifications. The one or more nucleic acid modifications may comprise a methylation modification, a phosphate modification, or a combination thereof. The modification may comprise a sugar modification, a sugar/backbone modification, a backbone modification, a base modification, an unnatural base pair, or a combination thereof. In some cases the sugar modification may comprise a 2′-fluoro, a 2′-O-methyl, a 2′-fluoro arabinose nucleic acid, a hexitol nucleic acid, a, 2′-O-methoxyethyl, a (1′-3′)-β-L-ribulo nucleic acid, a α-L-threose nucleic acid, a 3′-2′ phosphonomethyl-threosyl nucleic acid, a 2′-deoxyxylonucleic acid, a phosphorothioate, an alkyl phosphonate nucleic acid, a peptide nucleic acid, or a combination thereof.

The one or more modifications of the nucleic acid of the first probe may comprise one or more chemical reactive moieties. The one or more chemical reactive moieties of the nucleic acid of the first probe may comprise a chemoselective reagent. The one or more chemical reactive moieties may comprise of a tetrazine, an alkyne, an azide, a trans-cyclooctene, a maleimide, an N-hydroxysuccinimide ester, an amine, a carboxylic acid, a hydroxyl, a cyclopropenone, a thiol, a norbornene, or a combination thereof.

The one or more modifications of the nucleic acid of the first probe may be located at one or both ends of the first probe. The one or more modifications of the nucleic acid of the first probe may be located within the first probe. The first probe may comprise a nucleic acid, and the nucleic acid may comprise a modification at the 5′ end, the 3′end, or a combination thereof. In cases where the first probe comprises one or more modifications, each of the one or more modifications may be the same or may be different. In some cases, some of the one or more modifications are the same and some are different. For example, in cases where the first probe comprises a nucleic acid and the nucleic acid comprises two phosphorylation modification, the modifications may be considered the same. In some cases, where the first probe comprises a nucleic acid and the nucleic acid comprises one phosphorylation modification and one hydroxyl modification, the modifications may be considered the different. The one or more modifications of the nucleic acid of the first probe may comprise a 5′ phosphate group.

The first probe may be ligated to the third probe. The first probe may be ligated to the third probe at one or more locations. For example, one end of the first probe may be ligated to one end of the third probe and another end of the first probe may be ligated to another end of the third probe. The first probe may be ligated to the third probe as a result of the first probe and third probe binding to the first analyte. For example, the first and the third probe may bind to the first analyte and an end of the first probe may be directly adjacent to an end of the third probe, thereby enabling a ligation reaction between the first end of the first probe and the end of the third probe to occur. The first probe may be ligated to the third probe as a result of the first probe and third probe binding to the second probe. For example, the first and the third probe may bind to the second probe and an end of the first probe may be directly adjacent to an end of the third probe, thereby enabling a ligation reaction between the end of the first probe and end of the third to occur. In some cases, the first probe may comprise a nucleic acid, wherein the nucleic acid may comprise a 5′ end and a 3′ end. In some cases, the third probe may comprise a nucleic acid, wherein the nucleic acid may comprise a 5′ end and a 3′ end. The 3′ end of the first probe and the 5′ end of the third probe may be ligated to each other, the 5′ end of the first probe and the 3′ end of the third probe may be ligated to each other, or a combination thereof.

The first probe may be ligated to the third probe using a ligase. The ligase may be one or more ligases. In some cases, the ligase may comprise a mammalian ligase. In some cases, the ligase may be a bacterial ligase. The ligase may be a DNA ligase I, DNA ligase II, DNA ligase III, DNA ligase IV, or a combination thereof. In some cases, the ligase may comprise an RNA ligase. In some cases, the ligase may ligate a 3′nucleotide of one nucleic acid to a 5′ nucleotide of a different nucleic acid. In some cases, the ligase may ligate a 3′ end of a nucleic acid to a 5′ end of the same nucleic acid. For example, a nucleic acid may comprise a 5′ end and a 3′ end and the 5′ end and 3′ may be ligated to each other to form a circular nucleic acid. The ligase may ligate two nucleotides that are part of a double-stranded nucleic acid. In some cases, the double-stranded nucleic acid may comprise a nick, and the location of the nick may be ligated by the ligase. In some embodiments, the double-stranded nucleic acid may comprise a DNA/DNA duplex. In some embodiments, the double-stranded nucleic acid may comprise an RNA/DNA duplex. The ligase may comprise one or more of the following: T4 DNA ligase, SplintR ligase, T3 DNA ligase, T7 DNA ligase, E. coli DNA ligase, Taq ligase, RtcB ligase, or a combination thereof.

One or more ends of the first probe may be ligated to one or more ends of the third probe. An end of the first probe (e.g. a first end of the first probe) may be ligated to an end of the third probe (e.g. a third end of the third probe). The first end may be ligated to the third end. The first end may be ligated to another end of the third probe (e.g. a fourth end of the third probe). Another end of the first probe (e.g. a second end of the first probe) may be ligated to the third end. The second end may be ligated to the fourth end. The first end of the first probe may be ligated to the second end of the first probe.

An end of the first probe may be directly proximal to an end of the third probe when hybridized to an analyte within the sample. In some cases, the first probe may be hybridized to an analyte to form a double-stranded region comprising an end of the first probe. In some cases, the third probe may be hybridized to the same analyte as that bound by the first probe to form another double-stranded region comprising an end of the third probe. The double stranded region comprising the end of the first probe may be separated from the double stranded region comprising the end of the third probe by a gap comprising one or more nucleotides of a single-stranded portion of the first analyte. For example, the first probe and third probe may each comprise a nucleic acid and bind the first analyte to generate a first double-stranded region formed between the first probe and the first analyte and a second double-stranded region formed between the first analyte and the third probe. The first double-stranded region and second double-stranded region may be separated by a single-stranded region comprising a nucleic acid sequence of the first analyte that is not bound by either the first probe or the third probe. In this case, the single-stranded region would be considered a gap that separates the first probe from the third probe by at least one nucleotide. In some cases, one or more ends of the first probe are directly adjacent to one more ends of the third probe (e.g. the first probe and third probe may be ligated without any gap filling reaction). For example, the first probe and third probe may each comprise a nucleic acid and bind the first analyte to generate a first double-stranded region formed between the first probe and a second double-stranded region formed between the first analyte and the third probe. The first double-stranded region and second double-stranded region may not be separated by a single-stranded region comprising a nucleic acid sequence of the first analyte that is not bound by either the first probe or the third probe. In this case, the single-stranded region would comprise not comprise a gap that separates the first probe from the third probe by at least one nucleotide and may be considered directly adjacent (e.g. the first probe and third probe may be ligated without any gap filling reaction). In some cases, one or more ends of the first probe are separated by one or more nucleotides of a single-stranded region of the first analyte from one or more ends of the third probe when bound to the sample. A gap-filling reaction may be performed to fill in the nucleotides separating an end of the first probe and an end of the third probe. In some cases, the first end and the third end are not separated by any nucleotides of a single-stranded region of the first analyte when bound to the sample. In some cases, the first end and the third end are separated by one or more any nucleotides of a single-stranded region of the first analyte when bound to the sample. In some cases, the second end and the fourth end are not separated by any nucleotides of a single-stranded region of the first analyte when hybridized to the first analyte. In some cases, the second end and the fourth end are separated by one or more any nucleotides of a single-stranded region of the first analyte when bound to the sample.

The first probe may be ligated to the third probe using one or more chemical reactive moieties. In some cases, the third probe may comprise one or more chemical reactive moieties and the first probe may comprise one or more chemical reactive moieties. The one or more chemical reactive moieties of the third probe may react with the one or more chemical reactive moieties of the first probe, thereby ligating the third probe and the first probe. The first probe may comprise a first chemical reactive moiety at the first end. The third probe may comprise a second chemical reactive moiety at the third end. The first and/or second chemical reactive moieties may comprise a tetrazine, an alkyne, an azide, a trans-cyclooctene, a maleimide, an N-hydroxysuccinimide ester, an amine, a carboxylic acid, a hydroxyl, a cyclopropenone, a thiol, a norbornene, or a combination thereof. The ligating step may comprise the first chemical reactive moiety reacting with the second chemical reactive moiety. The first probe may comprise a third chemical reactive moiety at the second end. The third probe may comprise a fourth chemical reactive moiety at the fourth end. The third and/or fourth chemical reactive moieties may comprise a tetrazine, an alkyne, an azide, a trans-cyclooctene, a maleimide, an N-hydroxysuccinimide ester, an amine, a carboxylic acid, a hydroxyl, a cyclopropenone, a thiol, a norbornene, or a combination thereof. The ligating step may comprise the third chemical reactive moiety reacting with the fourth chemical reactive moiety.

The first probe may be ligated to the third probe to form a circular oligonucleotide. The circular oligonucleotide may comprise a circular nucleic acid. The circular nucleic acid may be single-stranded, double-stranded, or a combination thereof. The circular nucleic acid may bind to an analyte. In some cases, the analyte may be the first analyte. The circular nucleic acid may bind to a second probe. The circular nucleic acid may comprise at least one barcode. In some cases, the circular nucleic acid may comprise one or more barcodes. Each of the one or more barcodes of the circular nucleic acid may be the same or different. For example, a circular nucleic acid may comprise two barcodes, and the two barcodes may comprise the same nucleic acid sequence and be considered the same. In some cases, the circular nucleic acid may comprise two barcodes, and the sequences of the two barcodes may differ by one or more nucleotide and be considered different.

The first probe may comprise a binding site (e.g. a first binding site of the first probe). The binding site (e.g. a first binding site of the first probe) may be configured to bind to the first analyte. The binding site (e.g. a first binding site of the first probe) of the first probe may comprise a nucleic acid sequence. The nucleic acid sequence of the binding site (e.g. a first binding site of the first probe) of the first probe may be at least about 2, at least about 4, at least about 6, at least about 8, at least about 10, at least about 12, at least about 14, at least about 16, at least about 18, at least about 20, at least about 22, at least about 24, at least about 26, at least about 28, at least about 30, at least about 32, at least about 34, at least about 36, at least about 38, at least about 40, at least about 42, at least about 44, at least about 46, at least about 48, at least about 50, or more nucleotides in length. The nucleic acid sequence may be at most about 2, at most about 4, at most about 6, at most about 8, at most about 10, at most about 12, at most about 14, at most about 16, at most about 18, at most about 20, at most about 22, at most about 24, at most about 26, at most about 28, at most about 30, at most about 32, at most about 34, at most about 36, at most about 38, at most about 40, at most about 42, at most about 44, at most about 46, at most about 48, at most about 50, or less nucleotides in length. The nucleic acid sequence may be about 2 to about 50, about 4 to about 48, about 6 to about 46, about 8 to about 44, about 10 to about 42, about 12 to about 40, about 14 to about 38, about 16 to about 36, about 18 to about 34, about 20 to about 32, about 22 to about 30, or about 24 to about 28 nucleotides in length.

The first probe may comprise a second binding site. The second binding site may be configured to bind to the second probe. The second binding site of the first probe may comprise a nucleic acid. The nucleic acid of the second binding site of the first probe may be at least about 2, at least about 4, at least about 6, at least about 8, at least about 10, at least about 12, at least about 14, at least about 16, at least about 18, at least about 20, at least about 22, at least about 24, at least about 26, at least about 28, at least about 30, at least about 32, at least about 34, at least about 36, at least about 38, at least about 40, at least about 42, at least about 44, at least about 46, at least about 48, at least about 50, or more nucleotides in length. The nucleic acid may be at most about 2, at most about 4, at most about 6, at most about 8, at most about 10, at most about 12, at most about 14, at most about 16, at most about 18, at most about 20, at most about 22, at most about 24, at most about 26, at most about 28, at most about 30, at most about 32, at most about 34, at most about 36, at most about 38, at most about 40, at most about 42, at most about 44, at most about 46, at most about 48, at most about 50, or less nucleotides in length. The nucleic acid may be about 2 to about 50, about 4 to about 48, about 6 to about 46, about 8 to about 44, about 10 to about 42, about 12 to about 40, about 14 to about 38, about 16 to about 36, about 18 to about 34, about 20 to about 32, about 22 to about 30, or about 24 to about 28 nucleotides in length.

The second probe may comprise a binding site (e.g. a third binding site of the first probe). The binding site (e.g. a third binding site of the first probe) may be configured to bind to the first probe. The binding site (e.g. a third binding site of the first probe) may comprise a nucleic acid sequence. The nucleic acid sequence may be at least about 2, at least about 4, at least about 6, at least about 8, at least about 10, at least about 12, at least about 14, at least about 16, at least about 18, at least about 20, at least about 22, at least about 24, at least about 26, at least about 28, at least about 30, at least about 32, at least about 34, at least about 36, at least about 38, at least about 40, at least about 42, at least about 44, at least about 46, at least about 48, at least about 50, or more nucleotides in length. The nucleic acid sequence may be at most about 2, at most about 4, at most about 6, at most about 8, at most about 10, at most about 12, at most about 14, at most about 16, at most about 18, at most about 20, at most about 22, at most about 24, at most about 26, at most about 28, at most about 30, at most about 32, at most about 34, at most about 36, at most about 38, at most about 40, at most about 42, at most about 44, at most about 46, at most about 48, at most about 50, or less nucleotides in length. The nucleic acid sequence may be about 2 to about 50, about 4 to about 48, about 6 to about 46, about 8 to about 44, about 10 to about 42, about 12 to about 40, about 14 to about 38, about 16 to about 36, about 18 to about 34, about 20 to about 32, about 22 to about 30, or about 24 to about 28 nucleotides in length.

The second probe may comprise a fourth binding site. The fourth binding site may be configured to bind to the third probe. The fourth binding site may comprise a nucleic acid sequence. The nucleic acid sequence of the fourth binding site of the second probe may be at least about 2, at least about 4, at least about 6, at least about 8, at least about 10, at least about 12, at least about 14, at least about 16, at least about 18, at least about 20, at least about 22, at least about 24, at least about 26, at least about 28, at least about 30, at least about 32, at least about 34, at least about 36, at least about 38, at least about 40, at least about 42, at least about 44, at least about 46, at least about 48, at least about 50, or more nucleotides in length. The nucleic acid sequence may be at most about 2, at most about 4, at most about 6, at most about 8, at most about 10, at most about 12, at most about 14, at most about 16, at most about 18, at most about 20, at most about 22, at most about 24, at most about 26, at most about 28, at most about 30, at most about 32, at most about 34, at most about 36, at most about 38, at most about 40, at most about 42, at most about 44, at most about 46, at most about 48, at most about 50, or less nucleotides in length. The nucleic acid sequence may be about 2 to about 50, about 4 to about 48, about 6 to about 46, about 8 to about 44, about 10 to about 42, about 12 to about 40, about 14 to about 38, about 16 to about 36, about 18 to about 34, about 20 to about 32, about 22 to about 30, or about 24 to about 28 nucleotides in length.

The second probe may comprise a binding site (e.g. a fifth binding site of the second probe). The binding site (e.g. a fifth binding site of the second probe) may be configured to bind to the second analyte. The binding site (e.g. a fifth binding site of the second probe) may comprise a nucleic acid sequence. The nucleic acid sequence of the binding site (e.g. a fifth binding site of the second probe) of the second probe may be at least about 2, at least about 4, at least about 6, at least about 8, at least about 10, at least about 12, at least about 14, at least about 16, at least about 18, at least about 20, at least about 22, at least about 24, at least about 26, at least about 28, at least about 30, at least about 32, at least about 34, at least about 36, at least about 38, at least about 40, at least about 42, at least about 44, at least about 46, at least about 48, at least about 50, or more nucleotides in length. The nucleic acid sequence may be at most about 2, at most about 4, at most about 6, at most about 8, at most about 10, at most about 12, at most about 14, at most about 16, at most about 18, at most about 20, at most about 22, at most about 24, at most about 26, at most about 28, at most about 30, at most about 32, at most about 34, at most about 36, at most about 38, at most about 40, at most about 42, at most about 44, at most about 46, at most about 48, at most about 50, or less nucleotides in length. The nucleic acid sequence may be about 2 to about 50, about 4 to about 48, about 6 to about 46, about 8 to about 44, about 10 to about 42, about 12 to about 40, about 14 to about 38, about 16 to about 36, about 18 to about 34, about 20 to about 32, about 22 to about 30, or about 24 to about 28 nucleotides in length.

The binding site (e.g. a fifth binding site of the second probe) may comprise a polypeptide. The polypeptide of the binding site (e.g. a fifth binding site of the second probe) may comprise a protein or a portion thereof. In some cases, the polypeptide may comprise an antibody or antibody fragment. In some cases, the polypeptide may comprise a portion of an antibody or antibody fragment. In cases where the binding site (e.g. a fifth binding site of the second probe) of the second probe may comprise a polypeptide, the binding site (e.g. a fifth binding site of the second probe) may be connected to the fourth binding site of the second probe via a linker. The linker may comprise a variety of chemical groups including one or more ethylene groups, one or more methylene groups, one or more poly-ethylene glycol groups, or a combination thereof. The linker may connect the binding site (e.g. a fifth binding site of the second probe) of the second probe to the fourth binding site of the second probe through one or more covalent bonds. The linker may connect the binding site (e.g. a fifth binding site of the second probe) of the second probe to the fourth binding site of the second probe through one or more non-covalent bonds. In some cases, binding site (e.g. a fifth binding site of the second probe) of the second probe may be connected to the fourth binding site of the second probe through a combination of one or more covalent interactions and one or more non-covalent interactions. For example, in some cases, the binding site (e.g. a fifth binding site of the second probe) of the second probe may comprise a nucleic acid and the fourth binding site of the second probe may comprise a nucleic acid and the binding site (e.g. a fifth binding site of the second probe) and fourth binding site may be connected by one or more phosphodiester linkages. In some cases, the second probe may comprise an antibody and a nucleic acid, wherein the antibody may be connected to the nucleic acid through a linker. In some cases, the second probe may comprise an antibody that is configured to bind to the second analyte, and antibody that is configured to bind the antibody the binds the second analyte and a nucleic acid that is linked to the antibody that is configured to bind to the antibody that binds to the second analyte. In cases where the binding site (e.g. a fifth binding site of the second probe) of the second probe comprises a polypeptide, the polypeptide may be at least about 2, at least about 4, at least about 6, at least about 8, at least about 10, at least about 12, at least about 14, at least about 16, at least about 18, at least about 20, at least about 22, at least about 24, at least about 26, at least about 28, at least about 30, at least about 32, at least about 34, at least about 36, at least about 38, at least about 40, at least about 42, at least about 44, at least about 46, at least about 48, at least about 50, or more amino acids in length. The polypeptide may be at most about 2, at most about 4, at most about 6, at most about 8, at most about 10, at most about 12, at most about 14, at most about 16, at most about 18, at most about 20, at most about 22, at most about 24, at most about 26, at most about 28, at most about 30, at most about 32, at most about 34, at most about 36, at most about 38, at most about 40, at most about 42, at most about 44, at most about 46, at most about 48, at most about 50, or less amino acids in length. The polypeptide may be about 2 to about 50, about 4 to about 48, about 6 to about 46, about 8 to about 44, about 10 to about 42, about 12 to about 40, about 14 to about 38, about 16 to about 36, about 18 to about 34, about 20 to about 32, about 22 to about 30, or about 24 to about 28 amino acids in length.

The third probe may be configured to bind to a variety of components. In some cases, the third probe may bind to one or more analytes, one or more probes, or a combination thereof. The third probe may be configured to bind to the first analyte at one or more binding sites (e.g. the seventh binding site of the third probe). The third probe may be configured to bind to the second probe at one or more binding sites (e.g. the sixth binding site of the third probe).

The third probe may comprise a nucleic acid. The nucleic acid of the third probe may comprise DNA, RNA, or a combination thereof. The nucleic acid of the third probe may comprise single-stranded regions, double-stranded regions, or a combination thereof. The third probe may comprise one or more modifications. The one or more modifications of the third probe may be one or more ends of the third probe, internal of the third probe, or a combination thereof. In cases where the third probe comprises a nucleic acid, the one or more modifications may comprise one or more nucleic acid modifications. The one or more modifications of the nucleic acid of the third probe may comprise a sugar modification, a sugar/backbone modification, a backbone modification, a base modification, an unnatural base pair, or a combination thereof. In some cases the sugar modification may comprise a 2′-fluoro, a 2′-O-methyl, a 2′-fluoro arabinose nucleic acid, a hexitol nucleic acid, a, 2′-O-methoxyethyl, a (1′-3′)-β-L-ribulo nucleic acid, a α-L-threose nucleic acid, a 3′-2′ phosphonomethyl-threosyl nucleic acid, a 2′-deoxyxylonucleic acid, a phosphorothioate, an alkyl phosphonate nucleic acid, a peptide nucleic acid, or a combination thereof.

The third probe may recognize, couple to and/or bind to the first analyte. The first analyte may comprise a variety of analyte types and depending on the analyte type, the third probe may recognize a specific feature of the first analyte. For example, the third probe may recognize and bind to a ribonucleic acid and a specific feature of the ribonucleic acid may comprise a sequence, a secondary structure of the ribonucleic acid, a tertiary structure of the ribonucleic acid, or a combination thereof. The first analyte may comprise the ribonucleic acid. The ribonucleic acid may comprise a variety of types of RNA, including but not limited to messenger RNA (mRNA), ribosomal RNA (rRNA), transfer RNA (tRNA), micro RNA (miRNA), or a combination thereof. The RNA may be endogenous to the sample or exogenous to the sample. The RNA may have been introduced to the sample using variety of means, including but not limited to use of an AAV or rAAV. In cases where the second analyte comprises an RNA, the RNA may comprise one or more modifications. The one or more modifications of the first analyte may comprise an N 6-methyladenosine, a 5-methylcytosine, an N 1-methyladenosine, an N 7-methylguanosine, an N 4-acetylcytosine, a pseudouridine, an N1-methylpseudouridine, or a combination thereof. The RNA may comprise single-stranded regions, double-stranded regions, or a combination thereof. The RNA may comprise a plurality of nucleotides. The plurality of nucleotides may comprise A, C, U, G, or a combination thereof. In some cases, the RNA may comprise a secondary structure, a tertiary structure, or a combination thereof.

The third probe may recognize the first analyte. The first analyte that may be recognized by the third probe may comprises a nucleic acid. The nucleic acid of the first analyte may comprise one or more single nucleotide polymorphisms. The third probe may bind to the nucleic acid comprising the one or more single nucleotide polymorphisms. For example, the third probe may comprise a nucleic acid and hybridize to the nucleic acid comprising one or more single nucleotide polymorphisms. The binding of the third probe to the nucleic acid comprising the one or more single nucleotide polymorphisms may enable ligation of the third probe to the first probe. For example, binding of the third probe to the nucleic acid comprising the one or more single nucleotide polymorphisms may result in an end of the third probe being adjacent to an end of the first probe and the end of the third probe and end of the first probe may be ligated in the presence of a ligase. In some cases, the ligation of the third probe may comprise ligation of one end of the third probe to another end of the third probe. In some cases, the ligation of the third probe may comprise ligation of one end of the third probe to one end of the first probe.

In some cases, the third probe may not recognize an analyte (e.g. may not bind an analyte) when the analyte comprises a nucleic acid and the nucleic acid may comprise one or more single nucleotide polymorphism. The third probe may not bind to the nucleic acid comprising the one or more single nucleotide polymorphisms because the sequence of the one or more single nucleotide polymorphisms is different than the complement or reverse complement of the third probe. The third probe may not be ligated in the presence of the analyte described herein because the third probe may not bind the nucleic acid comprising one or more single nucleotide polymorphisms. In some cases, the third probe may not recognize (e.g. bind to) an analyte comprising a nucleic acid comprising one or more single nucleotide polymorphisms and because the third probe may not recognize the analyte, the third probe may not be ligated to the first probe if the analyte comprises the one or more single nucleotide polymorphism. The analyte may be the first analyte.

In some cases, the third probe may recognize the first analyte when the first analyte comprises a nucleic acid and the nucleic acid may comprise one or more modifications. The one or more modifications of the nucleic acid of the first analyte may comprise one or more methyl modifications. The one or more modifications of the nucleic acid of the first analyte may comprise a N 6-methyladenosine, a 5-methylcytosine, a N 1-methyladenosine, a N 7-methylguanosine, a N 4-acetylcytosine, a pseudouridine, a N1-methylpseudouridine, or a combination thereof. The third probe may bind to the nucleic acid comprising the one or more modifications. The binding of the third probe to the nucleic acid comprising the one or more modifications may enable ligation of the third probe to the first probe. For example, binding of the third probe to the nucleic acid comprising the one or more modifications may result in an end of the third probe being adjacent to an end of the first probe and the end of the third probe and end of the first probe may be ligated in the presence of a ligase. In some cases, the ligation of the third probe may comprise ligation of one end of the third probe to another end of the third probe. In some cases, the ligation of the third probe may comprise ligation of one end of the third probe to one end of the third probe.

In some cases, the third probe may not recognize an analyte when the analyte comprises a nucleic acid and the nucleic acid may comprise one or more modifications. The third probe may not bind to the nucleic acid comprising the one or more modifications because the sequence of the one or more modifications is different than what the third probe recognizes. The third probe may not be ligated in the presence of the analyte described herein. In some cases the third probe may not recognize an analyte comprising a nucleic acid comprising one or more modifications and the third probe is not ligated if the analyte comprises the one or more modifications. The analyte may be the first analyte.

For example, the third probe may recognize a DNA. The first probe may recognize, bind to, and/or couple to a DNA. The first analyte may comprise the DNA recognized by the third probe, the first probe, or a combination thereof. The first probe may recognize, bind to, and/or couple to a DNA. The DNA of the first analyte may be associated with one or more histone molecules (e.g. the DNA may form a complex with one or more histones). The DNA of the first analyte may be associated with one or more nucleosomes (e.g. the DNA may be wrapped around one or more histone molecules to form one or more nucleosomes). The DNA of the first analyte may be associated with a polymerase (e.g. the DNA may be interacting with a polymerase). The DNA may be associated with a DNA polymerase. The DNA of the first analyte may be endogenous to the sample (e.g. the DNA may be synthesized within one or more cells of the sample). The DNA of the first analyte may be exogenous to the sample (e.g. the DNA may be inserted into one or more cells of the sample from an external source). The DNA of the first analyte may be a combination of endogenous and exogenous DNA. The DNA of the first analyte may be single-stranded, double-stranded or a combination thereof. The DNA of the first analyte may a plurality of nucleotides. The plurality of nucleotides may comprise A, C, T, G, or a combination thereof. The DNA of the first analyte may comprise an oligonucleotide. The oligonucleotide may be single-stranded, double-stranded, or a combination thereof. In some cases, the oligonucleotide may comprise a secondary structure. In some cases, the DNA of the first analyte may comprise a secondary structure, a tertiary structure or a combination thereof. The DNA of the first analyte may comprise a modification. The Modification may comprise a methyl modification.

The third probe may comprise one or more barcodes. The one or more barcodes may comprise a nucleic acid. The nucleic acid of the third probe may comprise a combination of nucleotides including but not limited to A, C, G, T, U, or a combination thereof.

The one or more barcodes described herein may provide information related to one or more analytes. For example, the information related to the first analyte, the second analyte, or a combination thereof may be sequence information, location information, expression level information, proximity information, or a combination thereof. The barcode may denote the proximity of the first analyte to the second analyte. For example, detection of the barcode may indicate that the first analyte is within 100-1000 nm of the second analyte. The barcode may be adjacent to the binding site (e.g. a first binding site of the first probe), the second binding site, or a combination thereof. For example, the barcode of the first probe may comprise nucleic acid and the sequence of the nucleic acid may be within 0-25 nucleotides of the binding site (e.g. a first binding site of the first probe) of the first probe. The binding site (e.g. a first binding site of the first probe) may comprise the barcode or a portion thereof. For example, the barcode of the first probe may comprise a nucleic acid sequence and the binding site (e.g. a first binding site of the first probe) of the first probe may comprise a nucleic acid sequence and a portion of the nucleic acid sequence of the barcode and the nucleic acid sequence of the binding site (e.g. a first binding site of the first probe) may be the same nucleic acid sequence. The second binding site may comprise the barcode or a portion thereof. For example, the barcode of the first probe may comprise a nucleic acid sequence and the second binding site of the first probe may comprise a nucleic acid sequence and a portion of the nucleic acid sequence of the barcode and the nucleic acid sequence of the second binding site may be the same nucleic acid sequence.

The one or more barcodes of the third probe may provide information related to the first analyte, the second analyte, a third analyte or a combination thereof. For example, the information related to the first analyte, the second analyte, or a combination thereof may be sequence information, location information, expression level information, proximity information, or a combination thereof. The barcode of the third probe may denote the proximity of the first analyte to the second analyte. For example, detection of the barcode may indicate that the first analyte is within 100-1000 nm of the second analyte. The one or more barcodes may be adjacent to the sixth binding site, the seventh binding site, or a combination thereof. For example, the barcode of the third probe may comprise a nucleic acid and the sequence of the nucleic acid of the barcode of the third probe may be within 0-25 nucleotides of the sixth binding site of the first probe. The sixth binding site of the third probe may comprise the barcode or a portion thereof. For example, a portion of the sequence of a nucleic acid of the barcode may be the same sequence as a portion of the sequence of a nucleic acid of the sixth binding site. The seventh binding site may comprise the barcode or a portion thereof. Each of the one or more barcodes may comprise a variety of lengths, including but not limited to at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 51, at least about 52, at least about 53, at least about 54, at least about 55, at least about 56, at least about 57, at least about 58, at least about 59, at least about 60, at least about 61, at least about 62, at least about 63, at least about 64, at least about 65, at least about 66, at least about 67, at least about 68, at least about 69, at least about 70, at least about 71, at least about 72, at least about 73, at least about 74, at least about 75, at least about 76, at least about 77, at least about 78, at least about 79, at least about 80, at least about 81, at least about 82, at least about 83, at least about 84, at least about 85, at least about 86, at least about 87, at least about 88, at least about 89, at least about 90, at least about 91, at least about 92, at least about 93, at least about 94, at least about 95, at least about 96, at least about 97, at least about 98, at least about 99, at least about 100, at least about 105, at least about 110, at least about 115, at least about 120, at least about 125, at least about 130, at least about 135, at least about 140, at least about 145, at least about 150, at least about 155, at least about 160, at least about 165, at least about 170, at least about 175, at least about 180, at least about 185, at least about 190, at least about 195, at least about 200, or more nucleotides. The one or more barcodes may have a length of at most about 1, at most about 2, at most about 3, at most about 4, at most about 5, at most about 6, at most about 7, at most about 8, at most about 9, at most about 10, at most about 11, at most about 12, at most about 13, at most about 14, at most about 15, at most about 16, at most about 17, at most about 18, at most about 19, at most about 20, at most about 21, at most about 22, at most about 23, at most about 24, at most about 25, at most about 26, at most about 27, at most about 28, at most about 29, at most about 30, at most about 31, at most about 32, at most about 33, at most about 34, at most about 35, at most about 36, at most about 37, at most about 38, at most about 39, at most about 40, at most about 41, at most about 42, at most about 43, at most about 44, at most about 45, at most about 46, at most about 47, at most about 48, at most about 49, at most about 50, at most about 51, at most about 52, at most about 53, at most about 54, at most about 55, at most about 56, at most about 57, at most about 58, at most about 59, at most about 60, at most about 61, at most about 62, at most about 63, at most about 64, at most about 65, at most about 66, at most about 67, at most about 68, at most about 69, at most about 70, at most about 71, at most about 72, at most about 73, at most about 74, at most about 75, at most about 76, at most about 77, at most about 78, at most about 79, at most about 80, at most about 81, at most about 82, at most about 83, at most about 84, at most about 85, at most about 86, at most about 87, at most about 88, at most about 89, at most about 90, at most about 91, at most about 92, at most about 93, at most about 94, at most about 95, at most about 96, at most about 97, at most about 98, at most about 99, at most about 100, at most about 105, at most about 110, at most about 115, at most about 120, at most about 125, at most about 130, at most about 135, at most about 140, at most about 145, at most about 150, at most about 155, at most about 160, at most about 165, at most about 170, at most about 175, at most about 180, at most about 185, at most about 190, at most about 195, at most about 200, or fewer nucleotides. The one or more barcodes may have a length of about 1 to about 200, about 2 to about 195, about 3 to about 190, about 4 to about 185, about 5 to about 180, about 6 to about 175, about 7 to about 170, about 8 to about 165, about 9 to about 160, about 10 to about 155, about 11 to about 150, about 12 to about 145, about 13 to about 140, about 14 to about 135, about 15 to about 130, about 16 to about 125, about 17 to about 120, about 18 to about 115, about 19 to about 110, about 20 to about 105, about 21 to about 100, about 22 to about 99, about 23 to about 98, about 24 to about 97, about 25 to about 96, about 26 to about 95, about 27 to about 94, about 28 to about 93, about 29 to about 92, about 30 to about 91, about 31 to about 90, about 32 to about 89, about 33 to about 88, about 34 to about 87, about 35 to about 86, about 36 to about 85, about 37 to about 84, about 38 to about 83, about 39 to about 82, about 40 to about 81, about 41 to about 80, about 42 to about 79, about 43 to about 78, about 44 to about 77, about 45 to about 76, about 46 to about 75, about 47 to about 74, about 48 to about 73, about 49 to about 72, about 50 to about 71, about 51 to about 70, about 52 to about 69, about 53 to about 68, about 54 to about 67, about 55 to about 66, about 56 to about 65, about 57 to about 64, about 58 to about 63, about 59 to about 62, or about 60 to about 61 nucleotides.

The third probe may comprise a binding site (e.g. a sixth binding site of the third probe). The binding site (e.g. a sixth binding site of the third probe) may be configured to bind to the second probe (e.g. hybridize to). The binding site (e.g. a sixth binding site of the third probe) of the third probe may comprise a nucleic acid sequence. The nucleic acid sequence of the binding site (e.g. a sixth binding site of the third probe) of the third probe may be at least about 2, at least about 4, at least about 6, at least about 8, at least about 10, at least about 12, at least about 14, at least about 16, at least about 18, at least about 20, at least about 22, at least about 24, at least about 26, at least about 28, at least about 30, at least about 32, at least about 34, at least about 36, at least about 38, at least about 40, at least about 42, at least about 44, at least about 46, at least about 48, at least about 50, or more nucleotides in length. The nucleic acid sequence may be at most about 2, at most about 4, at most about 6, at most about 8, at most about 10, at most about 12, at most about 14, at most about 16, at most about 18, at most about 20, at most about 22, at most about 24, at most about 26, at most about 28, at most about 30, at most about 32, at most about 34, at most about 36, at most about 38, at most about 40, at most about 42, at most about 44, at most about 46, at most about 48, at most about 50, or less nucleotides in length. The nucleic acid sequence may be about 2 to about 50, about 4 to about 48, about 6 to about 46, about 8 to about 44, about 10 to about 42, about 12 to about 40, about 14 to about 38, about 16 to about 36, about 18 to about 34, about 20 to about 32, about 22 to about 30, or about 24 to about 28 nucleotides in length.

The third probe may comprise a binding site (e.g. a seventh binding site of the third probe). The binding site (e.g. a seventh binding site of the third probe) of the third probe may be configured to bind to the first analyte or a potion thereof. The binding site (e.g. a seventh binding site of the third probe) of the third probe may comprise a nucleic acid sequence. The nucleic acid sequence of the binding site (e.g. a seventh binding site of the third probe) of the third probe may be at least about 2, at least about 4, at least about 6, at least about 8, at least about 10, at least about 12, at least about 14, at least about 16, at least about 18, at least about 20, at least about 22, at least about 24, at least about 26, at least about 28, at least about 30, at least about 32, at least about 34, at least about 36, at least about 38, at least about 40, at least about 42, at least about 44, at least about 46, at least about 48, at least about 50, or more nucleotides in length. The nucleic acid sequence may be at most about 2, at most about 4, at most about 6, at most about 8, at most about 10, at most about 12, at most about 14, at most about 16, at most about 18, at most about 20, at most about 22, at most about 24, at most about 26, at most about 28, at most about 30, at most about 32, at most about 34, at most about 36, at most about 38, at most about 40, at most about 42, at most about 44, at most about 46, at most about 48, at most about 50, or less nucleotides in length. The nucleic acid sequence may be about 2 to about 50, about 4 to about 48, about 6 to about 46, about 8 to about 44, about 10 to about 42, about 12 to about 40, about 14 to about 38, about 16 to about 36, about 18 to about 34, about 20 to about 32, about 22 to about 30, or about 24 to about 28 nucleotides in length.

Another aspect of the disclosure provides a method for detecting analytes in a sample. The method may comprise providing the sample. The sample may comprise a first analyte and/or a second analyte. The first analyte may comprise a genetic aberration. The first analyte may be contacted with a first binding agent. The first binding agent may comprise a barcode. A reverse complement of the barcode may be detected with greater than 90% accuracy. The reverse complement of the barcode may be produced only when the first analyte is proximal to the second analyte.

The methods described herein also may have certain advantages that result in a high accuracy of detection. For example, the first probe and second probe need to bind to each other as well as the first probe binding the first analyte and the second probe binding the second analyte. The combination of these binding events may enable a ligation event between one end of the first probe to another end of the first probe. The ligation event may enable an amplification reaction to be performed. The amplification reaction may result in the formation of an amplicon with multiple copies of a barcode or reverse complement of a barcode that may be detected. Detection of the signal associated with the proximity of one analyte to another analyte may require multiple steps to be performed and minimize the chances of spurious or non-specific signal to be generated. As a result, the accuracy of detection may be higher than other methods. The accuracy the detection methods described herein also may have high accuracy for differentiating between analytes that have a modification or do not have a modification due to the specificity of the ligation reaction. The accuracy the detection methods described herein also may have high accuracy for differentiating between analytes that have a single nucleotide polymorphism or do not have a single nucleotide polymorphism due to the specificity of the ligation reaction. The accuracy of detection may be measure by a variety of metrics, including but not limited to specificity, sensitivity, detecting the correct barcode or complement thereof, detecting the correct barcode or complement thereof in comparison to detecting incorrect barcodes or complements thereof, or a combination thereof. In some cases, the accuracy of detection may be at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100%. In some cases, the accuracy of detection may be at most about 50%, at most about 51%, at most about 52%, at most about 53%, at most about 54%, at most about 55%, at most about 56%, at most about 57%, at most about 58%, at most about 59%, at most about 60%, at most about 61%, at most about 62%, at most about 63%, at most about 64%, at most about 65%, at most about 66%, at most about 67%, at most about 68%, at most about 69%, at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 96%, at most about 97%, at most about 98%, at most about 99%, or at most about 100% accurate.

The first analyte may comprise one or more genetic aberrations. Each of the one or more genetic aberrations of the first analyte may be the same or different. For example, the first analyte may comprise two single nucleotide polymorphisms, and one single nucleotide polymorphisms may comprise an A to T substitution and the other may comprise an A to C substitution. These single nucleotide polymorphisms may be considered different. The one or more genetic aberration may comprise one or more insertions, one or more deletions, one or more single nucleotide polymorphisms, one or more single nucleotide variations, one or more copy number variations, or a combination thereof.

The first analyte may comprise a nucleic acid. The nucleic acid may comprise RNA, DNA, or a combination thereof. In some cases, the first analyte may comprise RNA and the genetic aberration may comprise one or more RNA modifications. The one or more RNA modifications may comprise N 6-methyladenosine, 5-methylcytosine, N 1-methyladenosine, N 7-methylguanosine, N 4-acetylcytosine, pseudouridine, N1-methylpseudouridine, or a combination thereof. In some cases, the first analyte may comprise DNA and the genetic aberration may comprise one or more DNA modifications. The one or more DNA modifications may comprise a methyl modification.

The nucleic acid of the first analyte may comprise one or more single nucleotide polymorphisms. The one or more single nucleotide polymorphisms of the nucleic acid of the first analyte may comprise one or more variants relative to a reference genome. The reference genome may comprise a human reference genome, a mouse reference genome, a synthetic reference genome or a combination thereof. The human reference genome may comprise the NCBI Build 34 sequence, the NCBI Build 35 sequence, the NCBI Build 36.1 sequence, the GRCh37 sequence, the GRCh38 sequence, the T2T-CHM13 sequence, the GRCh39 sequence, the hg16 sequence, the hg17 sequence, the hg18 sequence, the hg19 sequence, the hs l sequence, or a combination thereof. The one or more single nucleotide polymorphisms of the nucleic acid of the first analyte may comprise an A to T substitution, an A to C substitution, an A to G substitution, a T to A substitution, a T to C substitution, a T to G substitution, a C to A substitution, a C to T substitution, a C to G substitution, a G to A substitution, a G to C substitution, a G to T substitution, or a combination thereof.

The nucleic acid of the first analyte may comprise one or more single nucleotide variants. The one or more single nucleotide variants of the nucleic acid of the first analyte may comprise one or more variants relative to a reference genome. The reference genome may comprise a human reference genome, a mouse reference genome, a synthetic reference genome or a combination thereof. The human reference genome may comprise the NCBI Build 34 sequence, the NCBI Build 35 sequence, the NCBI Build 36.1 sequence, the GRCh37 sequence, the GRCh38 sequence, the T2T-CHM13 sequence, the GRCh39 sequence, the hg16 sequence, the hg17 sequence, the hg18 sequence, the hg19 sequence, the hsl sequence, or a combination thereof. The one or more single nucleotide variants of the nucleic acid of the first analyte may comprise an A to T substitution, an A to C substitution, an A to G substitution, a T to A substitution, a T to C substitution, a T to G substitution, a C to A substitution, a C to T substitution, a C to G substitution, a G to A substitution, a G to C substitution, a G to T substitution, or a combination thereof.

The reverse complement of the barcode may be formed from an amplification reaction of the barcode to form an amplicon. The amplification reaction may comprise generating one or more copy of the barcode or the reverse complement of the barcode. The amplification reaction may comprise rolling circle amplification. The rolling circle amplification may be performed using a circular nucleic acid and the circular nucleic acid may comprise one or more copies of the barcode. The amplicon formed from an amplification reaction may have one or more copies of a reverse complement of the one or more barcodes of the circular oligonucleotide. The amplicon formed from an amplification reaction may have at least 1 copy, at least 2 copies, at least 3 copies, at least about 4 copies, at least about 5 copies, at least about 6 copies, at least about 7 copies, at least about 8 copies, at least about 9 copies, at least about 10 copies, at least about 20 copies, at least about 30 copies, at least about 40 copies, at least about 50 copies, at least about 60 copies, at least about 70 copies, at least about 80 copies, at least about 90 copies, at least about 100 copies, at least about 135 copies, at least about 150 copies, at least about 175 copies, at least about 200 copies, at least about 300 copies, at least about 400 copies, at least about 500 copies, at least about 600 copies, at least about 700 copies, at least about 800 copies, at least about 900 copies, at least about 1000 copies, at least about 2000 copies, at least about 3000 copies, at least about 4000 copies, at least about 5000 copies, at least about 6000 copies, at least about 7000 copies, at least about 8000 copies, at least about 9000 copies, at least about 10000 copies, at least about 50000 copies, at least about 100000 copies, at least about 500000 copies, at least about 1000000, or more copies of a reverse complement of the one or more barcodes of the circular oligonucleotide. The amplicon formed from an amplification reaction may have one or more copies of a reverse complement of the one or more barcodes of the circular oligonucleotide. The amplicon formed from an amplification reaction may have at most 1 copy, at most 2 copies, at most 3 copies, at most about 4 copies, at most about 5 copies, at most about 6 copies, at most about 7 copies, at most about 8 copies, at most about 9 copies, at most about 10 copies, at most about 20 copies, at most about 30 copies, at most about 40 copies, at most about 50 copies, at most about 60 copies, at most about 70 copies, at most about 80 copies, at most about 90 copies, at most about 100 copies, at most about 135 copies, at most about 150 copies, at most about 175 copies, at most about 200 copies, at most about 300 copies, at most about 400 copies, at most about 500 copies, at most about 600 copies, at most about 700 copies, at most about 800 copies, at most about 900 copies, at most about 1000 copies, at most about 2000 copies, at most about 3000 copies, at most about 4000 copies, at most about 5000 copies, at most about 6000 copies, at most about 7000 copies, at most about 8000 copies, at most about 9000 copies, at most about 10000 copies, at most about 50000 copies, at most about 100000 copies, at most about 500000 copies, at most about 1000000, or fewer copies of a reverse complement of the one or more barcodes of the circular oligonucleotide. The amplicon formed from an amplification reaction may have about 1 to about 1000000 copies, about 2 to about 500000 copies, about 3 to about 100000 copies, about 4 to about 50000 copies, about 5 to about 10000 copies, about 6 to about 9000 copies, about 7 to about 8000 copies, about 8 to about 7000 copies, about 9 to about 6000 copies, about 10 to about 5000 copies, about 20 to about 4000 copies, about 30 to about 3000 copies, about 40 to about 2000 copies, about 50 to about 1000 copies, about 60 to about 900 copies, about 70 to about 800 copies, about 80 to about 700 copies, about 90 to about 600 copies, about 100 to about 500 copies, about 135 to about 400 copies, about 150 to about 300 copies, about 175 to about 200 copies of a reverse complement of the one or more barcodes of the circular oligonucleotide.

The present disclosure relates to detecting the proximity of one or more analytes to each other. The proximity of the one or more analytes may refer to one analyte being spatially close to another analyte. The proximity of one analyte relative to another analyte may refer to a subcellular co-localization. The proximity of one analyte relative to another analyte may refer to an extracellular co-localization. The proximity of the one or more analytes enables a ligation event to occur between one end of one probe to another end of either the same probe or another probe. The ligation event and downstream steps may allow for detection of the proximity of one analyte relative to another analyte. In some cases, on analyte may be too far from another analyte to enable a ligation event to occur between one end of one probe to another end of either the same probe or another probe. The proximity of analyte interactions described here may refer to interactions on the scale of at most about 1 nanometer (nm), at most about 2 nm, at most about 3 nm, at most about 4 nm, at most about 5 nm, at most about 6 nm, at most about 7 nm, at most about 8 nm, at most about 9 nm, at most about 10 nm, at most about 15 nm, at most about 20 nm, at most about 25 nm, at most about 30 nm, at most about 35 nm, at most about 40 nm, at most about 45 nm, at most about 50 nm, at most about 60 nm, at most about 70 nm, at most about 80 nm, at most about 90 nm, at most about 100 nm, at most about 110 nm, at most about 120 nm, at most about 130 nm, at most about 140 nm, at most about 150 nm, at most about 160 nm, at most about 170 nm, at most about 180 nm, at most about 190 nm, at most about 200 nm, at most about 300 nm, at most about 400 nm, at most about 500 nm, at most about 600 nm, at most about 700 nm, at most about 800 nm, at most about 900 nm, at most about 1000 nm, or less. The proximity of analyte interactions described here may refer to interactions on the scale of at least about 1 nm, at least about 2 nm, at least about 3 nm, at least about 4 nm, at least about 5 nm, at least about 6 nm, at least about 7 nm, at least about 8 nm, at least about 9 nm, at least about 10 nm, at least about 15 nm, at least about 20 nm, at least about 25 nm, at least about 30 nm, at least about 35 nm, at least about 40 nm, at least about 45 nm, at least about 50 nm, at least about 60 nm, at least about 70 nm, at least about 80 nm, at least about 90 nm, at least about 100 nm, at least about 110 nm, at least about 120 nm, at least about 130 nm, at least about 140 nm, at least about 150 nm, at least about 160 nm, at least about 170 nm, at least about 180 nm, at least about 190 nm, at least about 200 nm, at least about 300 nm, at least about 400 nm, at least about 500 nm, at least about 600 nm, at least about 700 nm, at least about 800 nm, at least about 900 nm, at least about 1000 nm, or more.

The methods described herein also relate to detecting the proximity of analytes within a sample. The sample may be a variety of formats and may comprise a variety or features and/or characteristics. The sample may be extracted from a subject. The subject that the sample may be extracted from may be a human subject. The subject that the sample may be extracted from may be a non-human subject. The non-human subject may be a rat, a mouse, a non-human primate, a fruit fly, a zebrafish, or a combination thereof. The sample may comprise one or more cells. In some embodiments, the sample may comprise one or more cells, one or more tissue samples, one or more bodily fluids, or a combination thereof. The cells of the sample may be cultured cells. The cultured cells may be cultured in vivo, ex vivo or in vitro. The sample may comprise a tissue sample. The tissue sample may be fresh, fresh-frozen, fixed, fixed-frozen, formalin-fixed, paraffin embedded, or a combination thereof. In some cases, the tissue sample may be fixed using a cross-linking reagent. In some cases, the tissue sample may be fixed using a preservative. In some cases, the cross-linking reagent may comprise formaldehyde, formalin, glutaraldehyde, or a combination thereof.

The sample may comprise a tissue sample that has been sliced from a tissue block. The tissue sample may be immobilized onto a substrate. The substrate may be a well-plate, a slide, a coverslip, a well, a surface, a flow cell, or a combination thereof. The slide may be a microscope slide. The tissue sample may have a variety of thicknesses. The tissue sample may be at least at least about 1 μm thick, at least about 2 μm thick, at least about 3 μm thick, at least about 4 μm thick, at least about 5 μm thick, at least about 6 μm thick, at least about 7 μm thick, at least about 8 μm thick, at least about 9 μm thick, at least about 10 μm thick, at least about 11 μm thick, at least about 12 μm thick, at least about 13 μm thick, at least about 14 μm thick, at least about 15 μm thick, at least about 16 μm thick, at least about 17 μm thick, at least about 18 μm thick, at least about 19 μm thick, at least about 20 μm thick, at least about 21 μm thick, at least about 22 μm thick, at least about 23 μm thick, at least about 24 μm thick, at least about 25 μm thick, at least about 26 μm thick, at least about 27 μm thick, at least about 28 μm thick, at least about 29 μm thick, at least about 30 μm thick, at least about 31 μm thick, at least about 32 μm thick, at least about 33 μm thick, at least about 34 μm thick, at least about 35 μm thick, at least about 36 μm thick, at least about 37 μm thick, at least about 38 μm thick, at least about 39 μm thick, at least about 40 μm thick, at least about 41 μm thick, at least about 42 μm thick, at least about 43 μm thick, at least about 44 μm thick, at least about 45 μm thick, at least about 46 μm thick, at least about 47 μm thick, at least about 48 μm thick, at least about 49 μm thick, at least about 50 μm thick, at least about 51 μm thick, at least about 52 μm thick, at least about 53 μm thick, at least about 54 μm thick, at least about 55 μm thick, at least about 56 μm thick, at least about 57 μm thick, at least about 58 μm thick, at least about 59 μm thick, at least about 60 μm thick, at least about 61 μm thick, at least about 62 μm thick, at least about 63 μm thick, at least about 64 μm thick, at least about 65 μm thick, at least about 66 μm thick, at least about 67 μm thick, at least about 68 μm thick, at least about 69 μm thick, at least about 70 μm thick, at least about 71 μm thick, at least about 72 μm thick, at least about 73 μm thick, at least about 74 μm thick, at least about 75 μm thick, at least about 76 μm thick, at least about 77 μm thick, at least about 78 μm thick, at least about 79 μm thick, at least about 80 μm thick, at least about 81 μm thick, at least about 82 μm thick, at least about 83 μm thick, at least about 84 μm thick, at least about 85 μm thick, at least about 86 μm thick, at least about 87 μm thick, at least about 88 μm thick, at least about 89 μm thick, at least about 90 μm thick, at least about 91 μm thick, at least about 92 μm thick, at least about 93 μm thick, at least about 94 μm thick, at least about 95 μm thick, at least about 96 μm thick, at least about 97 μm thick, at least about 98 μm thick, at least about 99 μm thick, at least about 100 μm thick, at least about 105 μm thick, at least about 110 μm thick, at least about 115 μm thick, at least about 120 μm thick, at least about 125 μm thick, at least about 130 μm thick, at least about 135 μm thick, at least about 140 μm thick, at least about 145 μm thick, at least about 150 μm thick, at least about 155 μm thick, at least about 160 μm thick, at least about 165 μm thick, at least about 170 μm thick, at least about 175 μm thick, at least about 180 μm thick, at least about 185 μm thick, at least about 190 μm thick, at least about 195 μm thick, at least about 200 μm thick, at least about 210 μm thick, at least about 220 μm thick, at least about 230 μm thick, at least about 240 μm thick, at least about 250 μm thick, at least about 260 μm thick, at least about 270 μm thick, at least about 280 μm thick, at least about 290 μm thick, at least about 300 μm thick, at least about 320 μm thick, at least about 340 μm thick, at least about 360 μm thick, at least about 380 μm thick, at least about 400 μm thick, at least about 420 μm thick, at least about 440 μm thick, at least about 460 μm thick, at least about 480 μm thick, at least about 500 μm thick, or more. The tissue sample may be at most at most about 1 μm thick, at most about 2 μm thick, at most about 3 μm thick, at most about 4 μm thick, at most about 5 μm thick, at most about 6 μm thick, at most about 7 μm thick, at most about 8 μm thick, at most about 9 μm thick, at most about 10 μm thick, at most about 11 μm thick, at most about 12 μm thick, at most about 13 μm thick, at most about 14 μm thick, at most about 15 μm thick, at most about 16 μm thick, at most about 17 μm thick, at most about 18 μm thick, at most about 19 μm thick, at most about 20 μm thick, at most about 21 μm thick, at most about 22 μm thick, at most about 23 μm thick, at most about 24 μm thick, at most about 25 μm thick, at most about 26 μm thick, at most about 27 μm thick, at most about 28 μm thick, at most about 29 μm thick, at most about 30 μm thick, at most about 31 μm thick, at most about 32 μm thick, at most about 33 μm thick, at most about 34 μm thick, at most about 35 μm thick, at most about 36 μm thick, at most about 37 μm thick, at most about 38 μm thick, at most about 39 μm thick, at most about 40 μm thick, at most about 41 μm thick, at most about 42 μm thick, at most about 43 μm thick, at most about 44 μm thick, at most about 45 μm thick, at most about 46 μm thick, at most about 47 μm thick, at most about 48 μm thick, at most about 49 μm thick, at most about 50 μm thick, at most about 51 μm thick, at most about 52 μm thick, at most about 53 μm thick, at most about 54 μm thick, at most about 55 μm thick, at most about 56 μm thick, at most about 57 μm thick, at most about 58 μm thick, at most about 59 μm thick, at most about 60 μm thick, at most about 61 μm thick, at most about 62 μm thick, at most about 63 μm thick, at most about 64 μm thick, at most about 65 μm thick, at most about 66 μm thick, at most about 67 μm thick, at most about 68 μm thick, at most about 69 μm thick, at most about 70 μm thick, at most about 71 μm thick, at most about 72 μm thick, at most about 73 μm thick, at most about 74 μm thick, at most about 75 μm thick, at most about 76 μm thick, at most about 77 μm thick, at most about 78 μm thick, at most about 79 μm thick, at most about 80 μm thick, at most about 81 μm thick, at most about 82 μm thick, at most about 83 μm thick, at most about 84 μm thick, at most about 85 μm thick, at most about 86 μm thick, at most about 87 μm thick, at most about 88 μm thick, at most about 89 μm thick, at most about 90 μm thick, at most about 91 μm thick, at most about 92 μm thick, at most about 93 μm thick, at most about 94 μm thick, at most about 95 μm thick, at most about 96 μm thick, at most about 97 μm thick, at most about 98 μm thick, at most about 99 μm thick, at most about 100 μm thick, at most about 105 μm thick, at most about 110 μm thick, at most about 115 μm thick, at most about 120 μm thick, at most about 125 μm thick, at most about 130 μm thick, at most about 135 μm thick, at most about 140 μm thick, at most about 145 μm thick, at most about 150 μm thick, at most about 155 μm thick, at most about 160 μm thick, at most about 165 μm thick, at most about 170 μm thick, at most about 175 μm thick, at most about 180 μm thick, at most about 185 μm thick, at most about 190 μm thick, at most about 195 μm thick, at most about 200 μm thick, at most about 210 μm thick, at most about 220 μm thick, at most about 230 μm thick, at most about 240 μm thick, at most about 250 μm thick, at most about 260 μm thick, at most about 270 μm thick, at most about 280 μm thick, at most about 290 μm thick, at most about 300 μm thick, at most about 320 μm thick, at most about 340 μm thick, at most about 360 μm thick, at most about 380 μm thick, at most about 400 μm thick, at most about 420 μm thick, at most about 440 μm thick, at most about 460 μm thick, at most about 480 μm thick, at most about 500 μm thick, or less. The tissue sample may be about 1 to about 500 μm thick, about 2 to about 480 μm thick, about 3 to about 460 μm thick, about 4 to about 440 μm thick, about 5 to about 420 μm thick, about 6 to about 400 μm thick, about 7 to about 380 μm thick, about 8 to about 360 μm thick, about 9 to about 340 μm thick, about 10 to about 320 μm thick, about 11 to about 300 μm thick, about 12 to about 290 μm thick, about 13 to about 280 μm thick, about 14 to about 270 μm thick, about 15 to about 260 μm thick, about 16 to about 250 μm thick, about 17 to about 240 μm thick, about 18 to about 230 μm thick, about 19 to about 220 μm thick, about 20 to about 210 μm thick, about 21 to about 200 μm thick, about 22 to about 195 μm thick, about 23 to about 190 μm thick, about 24 to about 185 μm thick, about 25 to about 180 μm thick, about 26 to about 175 μm thick, about 27 to about 170 μm thick, about 28 to about 165 μm thick, about 29 to about 160 μm thick, about 30 to about 155 μm thick, about 31 to about 150 μm thick, about 32 to about 145 μm thick, about 33 to about 140 μm thick, about 34 to about 135 μm thick, about 35 to about 130 μm thick, about 36 to about 125 μm thick, about 37 to about 120 μm thick, about 38 to about 115 μm thick, about 39 to about 110 μm thick, about 40 to about 105 μm thick, about 41 to about 100 μm thick, about 42 to about 99 μm thick, about 43 to about 98 μm thick, about 44 to about 97 μm thick, about 45 to about 96 μm thick, about 46 to about 95 μm thick, about 47 to about 94 μm thick, about 48 to about 93 μm thick, about 49 to about 92 μm thick, about 50 to about 91 μm thick, about 51 to about 90 μm thick, about 52 to about 89 μm thick, about 53 to about 88 μm thick, about 54 to about 87 μm thick, about 55 to about 86 μm thick, about 56 to about 85 μm thick, about 57 to about 84 μm thick, about 58 to about 83 μm thick, about 59 to about 82 μm thick, about 60 to about 81 μm thick, about 61 to about 80 μm thick, about 62 to about 79 μm thick, about 63 to about 78 μm thick, about 64 to about 77 μm thick, about 65 to about 76 μm thick, about 66 to about 75 μm thick, about 67 to about 74 μm thick, about 68 to about 73 μm thick, about 69 to about 72 μm thick, or about 70 to about 71 μm thick. In some cases, the tissue sample is about 5 to about 250 μm thick, about 10 to about 100 μm thick, or about 25 to about 150 μm thick.

The sample may be embedded in a hydrogel. The hydrogel may be formed by polymerizing monomers in the presence of the sample. The hydrogel may comprise one or more polymers. The one or more polymers may comprise poly (vinyl alcohol) (PVA), poly (ethylene glycol) (PEG), poly (ethylene oxide) (PEO), poly (2-hydroxyethyl methacrylate) (PHEMA), poly (acrylic acid) (PAA), and poly (acrylamide) (PAAm), or a combination thereof. Hydrogel may be formed during any step of the methods described herein. For example, the hydrogel may be formed prior to contacting the sample with one or more probes. The hydrogel may be formed after contacting the sample with one or more probes. In some cases, the hydrogel may be formed after contacting the sample with one or more probes but before contacting the sample with one or more other probes. The hydrogel may be formed before an amplification step. The hydrogel may be formed after an amplification step. The hydrogel may be formed before a ligation step between one or more probes. The hydrogel may be formed after a ligation step between one or more probes. The hydrogel may be formed before a gap-filling reaction. The hydrogel may be formed after a gap-filling reaction. The hydrogel may be formed before a detection step. The hydrogel may be formed after a detection step. The sample may be embedded into a hydrogel. In some cases, the hydrogel may be formed prior to contacting the sample with one or more probe and the sample may be incubated prior to contacting the sample with one or more probes. The sample may be incubated for at least about 1 hour, at least about 12 hours, at least about a day, at least about two days, at least about three days, or longer before contacting the sample with one or more probes. The sample may be incubated for at most about 1 hour, at most about 12 hours, at most about a day, at most about two days, at most about three days, or less before contacting the sample with one or more probes.

The methods described herein also relate to detecting the proximity of analytes within a sample (e.g. determining whether an analyte is associated with or close to another analyte within a sample). One or more analytes may be analyzed to determine a proximity. In some cases, a variety of different types of analytes may be used for analysis, including but not limited to a nucleic acid, a polypeptide, a lipid, a small molecule, a cell, an intracellular feature, an extracellular feature, an exogenous feature, an endogenous feature, or a combination thereof. In some cases, one or more analytes of the same type may be analytes. For example, two different proteins may each be considered an analyte. In some cases, one or more analytes of different types of analytes may be analyzed. A protein may be analyzed for proximity to another protein. A protein may be analyzed for proximity to a nucleic acid. The nucleic acid that may be analyzed for proximity to a protein may comprise an RNA, a DNA, or a combination thereof. In some cases, a nucleic acid may be analyzed for proximity to another nucleic acid. An RNA may be analyzed for proximity to another RNA. In some cases, the other RNA may be a different region of the same RNA. For example, an RNA molecule may comprise two different portions, and each portion of the two different portions may be bound by one or more probes to assess proximity. An RNA may be analyzed for proximity to a DNA. A DNA may be analyzed for proximity to another DNA. In some cases, the DNA may be a different region of the same DNA. For example, a DNA molecule may comprise two different portions, and each of the two or more portions may be bound by one or more probes to assess proximity of the two or more portions of the DNA molecule. The proximity between the first analyte and the second analyte may be analyzed by detecting the presence of a barcode or complement thereof, which is amplified as a result of the first analyte and the second analyte being spatially localized to each other. The first analyte may comprise a variety of analyte types. The second analyte may comprise a variety of analyte types.

The first analyte may comprise a nucleic acid. The nucleic acid of the first analyte may comprise RNA, DNA, or a combination thereof. The first analyte may comprise one or more genetic aberrations. The first analyte may comprise an RNA. In cases where in the first analyte comprises an RNA and the RNA may comprise a variety of types of RNA, including but not limited to messenger RNA (mRNA), ribosomal RNA (rRNA), transfer RNA (tRNA), micro RNA (miRNA), or a combination thereof. The RNA may be endogenous to the sample or exogenous to the sample. The RNA may have been introduced to the sample using variety of means, including but not limited to use of an AAV or rAAV. In cases where the first analyte comprises an RNA, the RNA may comprise a modification. The modification may be an N 6-methyladenosine, a 5-methylcytosine, an N 1-methyladenosine, an N 7-methylguanosine, an N 4-acetylcytosine, a pseudouridine, an N1-methylpseudouridine, or a combination thereof. The RNA may comprise single-stranded regions, double-stranded regions, or a combination thereof. The RNA may comprise a plurality of nucleotides. The plurality of nucleotides may comprise A, C, U, G, or a combination thereof. In some cases, the RNA may comprise a secondary structure, a tertiary structure, or a combination thereof.

The first analyte may comprise a nucleic acid. The nucleic acid of the first analyte may comprise one or more genetic aberrations. The one or more genetic aberrations of the first analyte may comprise one or more insertions, one or more deletions, one or more copy number variations, one or more single nucleotide polymorphisms, one or more single nucleotide variants, or a combination thereof. The one or more single nucleotide polymorphisms of the nucleic acid of the first analyte may comprise one or more variants relative to a reference genome. The reference genome may comprise a human reference genome, a mouse reference genome, a synthetic reference genome or a combination thereof. The human reference genome may comprise the NCBI Build 34 sequence, the NCBI Build 35 sequence, the NCBI Build 36.1 sequence, the GRCh37 sequence, the GRCh38 sequence, the T2T-CHM13 sequence, the GRCh39 sequence, the hg16 sequence, the hg17 sequence, the hg18 sequence, the hg19 sequence, the hsl sequence, or a combination thereof. The one or more single nucleotide polymorphisms of the nucleic acid of the first analyte may comprise an A to T substitution, an A to C substitution, an A to G substitution, a T to A substitution, a T to C substitution, a T to G substitution, a C to A substitution, a C to T substitution, a C to G substitution, a G to A substitution, a G to C substitution, a G to T substitution, or a combination thereof. The one or more single nucleotide variants of the nucleic acid of the first analyte may comprise one or more variants relative to a reference genome. The reference genome may comprise a human reference genome, a mouse reference genome, a synthetic reference genome or a combination thereof. The human reference genome may comprise the NCBI Build 34 sequence, the NCBI Build 35 sequence, the NCBI Build 36.1 sequence, the GRCh37 sequence, the GRCh38 sequence, the T2T-CHM13 sequence, the GRCh39 sequence, the hg16 sequence, the hg17 sequence, the hg18 sequence, the hg19 sequence, the hs l sequence, or a combination thereof. The one or more single nucleotide variants may comprise an A to T substitution, an A to C substitution, an A to G substitution, a T to A substitution, a T to C substitution, a T to G substitution, a C to A substitution, a C to T substitution, a C to G substitution, a G to A substitution, a G to C substitution, a G to T substitution, or a combination thereof.

The first analyte may comprise a polypeptide. The polypeptide of the first analyte may comprise a protein, a peptide or a combination thereof. In cases where the polypeptide of the first analyte comprises a protein, the protein may comprise a transcription factor, a ribosomal protein, a histone, a polymerase, a helicase, a restriction enzyme, a ribonucleic acid binding protein, an enzyme, an antibody, a structural protein, a defense protein, a signaling protein, a receptor, a soluble protein, a transmembrane or a combination thereof. In cases where the protein may be a signaling protein, the signaling protein may be a cytokine, chemokine, or a combination thereof. In cases where the polypeptide of the first analyte comprises a protein, the protein may comprise a transcription factor, a ribosomal protein, a histone, a polymerase, or a combination thereof. In cases where the protein of the first analyte comprises a ribosomal protein, the ribosomal protein may comprise an S3A ribosomal protein, an SA ribosomal protein, an S3 ribosomal protein, an S9 ribosomal protein, an S4 (X, Y1, Y2) ribosomal protein, an S2 ribosomal protein, an S6 ribosomal protein, an S5 ribosomal protein, an S7 ribosomal protein, an S15A ribosomal protein, an S8 ribosomal protein, an S16 ribosomal protein, an S20 ribosomal protein, an S10 ribosomal protein, an S14 ribosomal protein, an S23 ribosomal protein, an S12 ribosomal protein, an S18 ribosomal protein, an S29 ribosomal protein, an S13 ribosomal protein, an S11 ribosomal protein, an S17 ribosomal protein, an S15 ribosomal protein, an S19 ribosomal protein, an S21 ribosomal protein, an S24 ribosomal protein, an S25 ribosomal protein, an S26 ribosomal protein, an S27 ribosomal protein, an S28 ribosomal protein, an S30 ribosomal protein, an S27A ribosomal protein, an RACK1 ribosomal protein, an L10A ribosomal protein, an L8 ribosomal protein, an L3 ribosomal protein, an L4 ribosomal protein, an L11 ribosomal protein, an L9 ribosomal protein, an L6 ribosomal protein, an L7A ribosomal protein, an P0 ribosomal protein, an L12 ribosomal protein, an L13A ribosomal protein, an L13 ribosomal protein, an L23 ribosomal protein, an L14 ribosomal protein, an L27A ribosomal protein, an L15 ribosomal protein, an L10 ribosomal protein, an L5 ribosomal protein, an L18 ribosomal protein, an L19 ribosomal protein, an L18A ribosomal protein, an L21 ribosomal protein, an L17 ribosomal protein, an L22 ribosomal protein, an L23A ribosomal protein, an L26 ribosomal protein, an L24 ribosomal protein, an L27 ribosomal protein, an L28 ribosomal protein, an L35 ribosomal protein, an L29 ribosomal protein, an L7 ribosomal protein, an L30 ribosomal protein, an L31 ribosomal protein, an L32 ribosomal protein, an L35A ribosomal protein, an L34 ribosomal protein, an L36 ribosomal protein, an L37 ribosomal protein, an L38 ribosomal protein, an L39 ribosomal protein, an L40 ribosomal protein, an L41 ribosomal protein, an L36A ribosomal protein, an L37A ribosomal protein, a P1/P2 (αβ) ribosomal protein, or a combination thereof.

The first analyte may comprise a protein and the protein may comprise one or more post translational modifications. The one or more post translational modifications of the protein of the first analyte may comprise a myristoylation, a palmitoylation, a farnesylation, a geranylgeranylation, a glypiation, a glycosylphosphatidylinositol, a lipoylation, a flavin moiety attachment, a heme C attachment, a phosphopantetheinylation, a retinylidene Schiff base formation, a modifications of translation factors, a diphthamide formation, a ethanolamine phosphoglycerol, a hypusine formation, a beta-Lysine addition on a lysine, a acylation (e.g. O-acylation, N-acylation, and S-acylation), an acetylation, a formylation, a alkylation, a amidation, a arginylation, a polyglutamylation, a polyglycylation, a butyrylation, a gamma-carboxylation, a glycosylation, a polysialylation, a malonylation, a hydroxylation, a nucleotide addition, a phosphate ester (O-linked), a phosphoramidate (N-linked) formation, a phosphorylation, a adenylylation, a uridylylation, a propionylation, a pyroglutamate formation, a S-glutathionylation, a S-nitrosylation, a S-sulfenylation, a S-sulfinylation, a S-sulfonylation, a succinylation, a sulfation, a glycation, a carbamylation, a carbonylation, a spontaneous isopeptide bond formation, a biotinylation, a carbamylation, an oxidation, a pegylation, an ubiquitination, a SUMOylation, a neddylation, an ISGylation, a citrullination, a deamidation, an eliminylation, or a combination thereof. The first analyte may comprise a chemical modification. The one or more post translational modifications of the protein of the first analyte may comprise an alkylation, a phosphorylation, or a combination thereof.

The second analyte may comprise a nucleic acid. The nucleic acid of the second analyte may comprise RNA, DNA, or a combination thereof. The second analyte may comprise one or more genetic aberrations. The second analyte may comprise an RNA. In some cases where the second analyte comprises an RNA, the RNA may comprise a variety of types of RNA, including but not limited to messenger RNA (mRNA), ribosomal RNA (rRNA), transfer RNA (tRNA), micro RNA (miRNA), or a combination thereof. The RNA of the second analyte may be endogenous to the sample or exogenous to the sample. The RNA may have been introduced to the sample using variety of means, including but not limited to use of an AAV or rAAV. In cases where the second analyte comprises an RNA, the RNA may comprise a modification. The modification may be an N 6-methyladenosine, a 5-methylcytosine, an N 1-methyladenosine, an N 7-methylguanosine, an N 4-acetylcytosine, a pseudouridine, an N1-methylpseudouridine, or a combination thereof. The RNA may comprise single-stranded regions, double-stranded regions, or a combination thereof. The RNA may comprise a plurality of nucleotides. The plurality of nucleotides may comprise A, C, U, G, or a combination thereof. In some cases, the RNA may comprise a secondary structure, a tertiary structure, or a combination thereof.

The second analyte may comprise a DNA. The DNA of the second analyte may be associated with one or more histone molecules (e.g. the DNA may form a complex with one or more histones). The DNA of the second analyte may be associated with one or more nucleosomes (e.g. the DNA may be wrapped around one or more histone molecules to form one or more nucleosomes). The DNA of the second analyte may be associated with a polymerase (e.g. the DNA may be interacting with a polymerase). The DNA of the second analyte may be associated with a DNA polymerase. The DNA of the second analyte may be endogenous to the sample (e.g. the DNA may be synthesized within one or more cells of the sample). The DNA of the second analyte may be exogenous to the sample (e.g. the DNA may be inserted into one or more cells of the sample from an external source). The DNA of the second analyte may be a combination of endogenous and exogenous DNA. The DNA of the second analyte may be single-stranded, double-stranded or a combination thereof. The DNA of the second analyte may a plurality of nucleotides. The plurality of nucleotides may comprise A, C, T, G, or a combination thereof. The DNA of the second analyte may comprise an oligonucleotide. The oligonucleotide may be single-stranded, double-stranded, or a combination thereof. In some cases, the oligonucleotide may comprise a secondary structure. In some cases, the DNA of the second analyte may comprise a secondary structure, a tertiary structure or a combination thereof. The DNA of the second analyte may comprise a modification. The Modification may comprise a methyl modification.

In cases where the second analyte comprises a nucleic acid, the nucleic acid may comprise one or more genetic aberrations. The one or more genetic aberrations of the second analyte may comprise one or more insertions, one or more deletions, one or more copy number variations, one or more single nucleotide polymorphisms, one or more single nucleotide variants, or a combination thereof. The one or more single nucleotide polymorphisms of the second analyte may comprise one or more variants relative to a reference genome. The reference genome may comprise a human reference genome, a mouse reference genome, a synthetic reference genome or a combination thereof. The human reference genome may comprise the NCBI Build 34 sequence, the NCBI Build 35 sequence, the NCBI Build 36.1 sequence, the GRCh37 sequence, the GRCh38 sequence, the T2T-CHM13 sequence, the GRCh39 sequence, the hg16 sequence, the hg17 sequence, the hg18 sequence, the hg19 sequence, the hsl sequence, or a combination thereof. The one or more single nucleotide polymorphisms of the second analyte may comprise an A to T substitution, an A to C substitution, an A to G substitution, a T to A substitution, a T to C substitution, a T to G substitution, a C to A substitution, a C to T substitution, a C to G substitution, a G to A substitution, a G to C substitution, a G to T substitution, or a combination thereof. The one or more single nucleotide variants of the second analyte may comprise one or more variants relative to a reference genome. The reference genome may comprise a human reference genome, a mouse reference genome, a synthetic reference genome or a combination thereof. The human reference genome may comprise the NCBI Build 34 sequence, the NCBI Build 35 sequence, the NCBI Build 36.1 sequence, the GRCh37 sequence, the GRCh38 sequence, the T2T-CHM13 sequence, the GRCh39 sequence, the hg16 sequence, the hg17 sequence, the hg18 sequence, the hg19 sequence, the hsl sequence, or a combination thereof. The one or more single nucleotide variants of the second analyte may comprise an A to T substitution, an A to C substitution, an A to G substitution, a T to A substitution, a T to C substitution, a T to G substitution, a C to A substitution, a C to T substitution, a C to G substitution, a G to A substitution, a G to C substitution, a G to T substitution, or a combination thereof.

The second analyte may comprise a polypeptide. The polypeptide of the second analyte may comprise a protein, a peptide or a combination thereof. In cases where the polypeptide of the second analyte comprises a protein, the protein may comprise a transcription factor, a ribosomal protein, a histone, a polymerase, a helicase, a restriction enzyme, a ribonucleic acid binding protein, an enzyme, an antibody, a structural protein, a defense protein, a signaling protein, a receptor, a soluble protein, a transmembrane or a combination thereof. In cases where the protein may be a signaling protein, the signaling protein may be a cytokine, chemokine, or a combination thereof. In cases where the polypeptide of the second analyte comprises a protein, the protein may comprise a transcription factor, a ribosomal protein, a histone, a polymerase, or a combination thereof. In cases where the protein of the second analyte comprises a ribosomal protein, the ribosomal protein may comprise an S3A ribosomal protein, an SA ribosomal protein, an S3 ribosomal protein, an S9 ribosomal protein, an S4 (X, Y1, Y2) ribosomal protein, an S2 ribosomal protein, an S6 ribosomal protein, an S5 ribosomal protein, an S7 ribosomal protein, an S15A ribosomal protein, an S8 ribosomal protein, an S16 ribosomal protein, an S20 ribosomal protein, an S10 ribosomal protein, an S14 ribosomal protein, an S23 ribosomal protein, an S12 ribosomal protein, an S18 ribosomal protein, an S29 ribosomal protein, an S13 ribosomal protein, an S11 ribosomal protein, an S17 ribosomal protein, an S15 ribosomal protein, an S19 ribosomal protein, an S21 ribosomal protein, an S24 ribosomal protein, an S25 ribosomal protein, an S26 ribosomal protein, an S27 ribosomal protein, an S28 ribosomal protein, an S30 ribosomal protein, an S27A ribosomal protein, an RACK1 ribosomal protein, an L10A ribosomal protein, an L8 ribosomal protein, an L3 ribosomal protein, an L4 ribosomal protein, an L11 ribosomal protein, an L9 ribosomal protein, an L6 ribosomal protein, an L7A ribosomal protein, an P0 ribosomal protein, an L12 ribosomal protein, an L13A ribosomal protein, an L13 ribosomal protein, an L23 ribosomal protein, an L14 ribosomal protein, an L27A ribosomal protein, an L15 ribosomal protein, an L10 ribosomal protein, an L5 ribosomal protein, an L18 ribosomal protein, an L19 ribosomal protein, an L18A ribosomal protein, an L21 ribosomal protein, an L17 ribosomal protein, an L22 ribosomal protein, an L23A ribosomal protein, an L26 ribosomal protein, an L24 ribosomal protein, an L27 ribosomal protein, an L28 ribosomal protein, an L35 ribosomal protein, an L29 ribosomal protein, an L7 ribosomal protein, an L30 ribosomal protein, an L31 ribosomal protein, an L32 ribosomal protein, an L35A ribosomal protein, an L34 ribosomal protein, an L36 ribosomal protein, an L37 ribosomal protein, an L38 ribosomal protein, an L39 ribosomal protein, an L40 ribosomal protein, an L41 ribosomal protein, an L36A ribosomal protein, an L37A ribosomal protein, a P1/P2 (αβ) ribosomal protein, or a combination thereof.

The second analyte may comprise a protein and the protein may comprise one or more post translational modifications. The one or more post translational modifications of the protein of the second analyte may comprise a myristoylation, a palmitoylation, a farnesylation, a geranylgeranylation, a glypiation, a glycosylphosphatidylinositol, a lipoylation, a flavin moiety attachment, a heme C attachment, a phosphopantetheinylation, a retinylidene Schiff base formation, a modifications of translation factors, a diphthamide formation, a ethanolamine phosphoglycerol, a hypusine formation, a beta-Lysine addition on a lysine, a acylation (e.g. O-acylation, N-acylation, and S-acylation), an acetylation, a formylation, a alkylation, a amidation, a arginylation, a polyglutamylation, a polyglycylation, a butyrylation, a gamma-carboxylation, a glycosylation, a polysialylation, a malonylation, a hydroxylation, a nucleotide addition, a phosphate ester (O-linked), a phosphoramidate (N-linked) formation, a phosphorylation, a adenylylation, a uridylylation, a propionylation, a pyroglutamate formation, a S-glutathionylation, a S-nitrosylation, a S-sulfenylation, a S-sulfinylation, a S-sulfonylation, a succinylation, a sulfation, a glycation, a carbamylation, a carbonylation, a spontaneous isopeptide bond formation, a biotinylation, a carbamylation, an oxidation, a pegylation, an ubiquitination, a SUMOylation, a neddylation, an ISGylation, a citrullination, a deamidation, an eliminylation, or a combination thereof. The second analyte may comprise a chemical modification. The one or more post translational modifications of the protein of the second analyte may comprise an alkylation, a phosphorylation, or a combination thereof.

The first probe may comprise a variety of components. In some cases, the first probe may comprise a nucleic acid. The nucleic acid of the first probe may comprise one or more deoxyribonucleic acids (DNA), ribonucleic acids (RNA), or a combination thereof. The nucleic acid of the first probe may comprise one or more modifications. In some cases, the one or more modifications of the nucleic acid of the first probe may comprise a DNA modification. In some cases, the one or more modifications may be an RNA modification. The one or more modifications may be located at one or both ends of the first probe, internal to the first probe, or a combination thereof. The one or more modifications of the first probe may be used for ligating the first probe to itself, ligating the first probe to another probe (e.g. the third probe), for detecting the first probe directly or indirectly, or a combination thereof. The one or more modifications may comprise a phosphate modification. In some cases, the phosphate modification may comprise a 5′ phosphate modification. In some cases, the phosphate modification may comprise a 3′ phosphate modification. The first probe may comprise a nucleic acid. The nucleic acid of the first probe may be an oligonucleotide. The oligonucleotide may comprise a modification. In some cases, the oligonucleotide may comprise a 5′ phosphate modification. The one or more modifications of the nucleic acid may comprise an internucleotide linkage. The internucleotide linkage of the nucleic acid of the first probe may comprise a phosphorothioate, a phosphodiester, or a combination thereof. The internucleotide linkage may comprise a locked nucleic acid (LNA). The internucleotide linkage may confer advantages to the methods described herein by increasing the melting temperature of oligonucleotide interactions, increasing stability of probe interactions, improving specificity of probe precognition, or a combination thereof.

The first probe may comprise a variety of lengths. In cases where the first probe comprises a nucleic acid, the nucleic acid may be at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 51, at least about 52, at least about 53, at least about 54, at least about 55, at least about 56, at least about 57, at least about 58, at least about 59, at least about 60, at least about 61, at least about 62, at least about 63, at least about 64, at least about 65, at least about 66, at least about 67, at least about 68, at least about 69, at least about 70, at least about 71, at least about 72, at least about 73, at least about 74, at least about 75, at least about 76, at least about 77, at least about 78, at least about 79, at least about 80, at least about 81, at least about 82, at least about 83, at least about 84, at least about 85, at least about 86, at least about 87, at least about 88, at least about 89, at least about 90, at least about 91, at least about 92, at least about 93, at least about 94, at least about 95, at least about 96, at least about 97, at least about 98, at least about 99, at least about 100, at least about 105, at least about 110, at least about 115, at least about 120, at least about 125, at least about 130, at least about 135, at least about 140, at least about 145, at least about 150, at least about 155, at least about 160, at least about 165, at least about 170, at least about 175, at least about 180, at least about 185, at least about 190, at least about 195, at least about 200, or more nucleotides in length. In cases where the first probe comprises a nucleic acid, the nucleic acid may be at most about 1, at most about 2, at most about 3, at most about 4, at most about 5, at most about 6, at most about 7, at most about 8, at most about 9, at most about 10, at most about 11, at most about 12, at most about 13, at most about 14, at most about 15, at most about 16, at most about 17, at most about 18, at most about 19, at most about 20, at most about 21, at most about 22, at most about 23, at most about 24, at most about 25, at most about 26, at most about 27, at most about 28, at most about 29, at most about 30, at most about 31, at most about 32, at most about 33, at most about 34, at most about 35, at most about 36, at most about 37, at most about 38, at most about 39, at most about 40, at most about 41, at most about 42, at most about 43, at most about 44, at most about 45, at most about 46, at most about 47, at most about 48, at most about 49, at most about 50, at most about 51, at most about 52, at most about 53, at most about 54, at most about 55, at most about 56, at most about 57, at most about 58, at most about 59, at most about 60, at most about 61, at most about 62, at most about 63, at most about 64, at most about 65, at most about 66, at most about 67, at most about 68, at most about 69, at most about 70, at most about 71, at most about 72, at most about 73, at most about 74, at most about 75, at most about 76, at most about 77, at most about 78, at most about 79, at most about 80, at most about 81, at most about 82, at most about 83, at most about 84, at most about 85, at most about 86, at most about 87, at most about 88, at most about 89, at most about 90, at most about 91, at most about 92, at most about 93, at most about 94, at most about 95, at most about 96, at most about 97, at most about 98, at most about 99, at most about 100, at most about 105, at most about 110, at most about 115, at most about 120, at most about 125, at most about 130, at most about 135, at most about 140, at most about 145, at most about 150, at most about 155, at most about 160, at most about 165, at most about 170, at most about 175, at most about 180, at most about 185, at most about 190, at most about 195, at most about 200, or less nucleotides in length. In cases where the first probe comprises a nucleic acid, the nucleic acid may be about 1 to about 200, about 2 to about 195, about 3 to about 190, about 4 to about 185, about 5 to about 180, about 6 to about 175, about 7 to about 170, about 8 to about 165, about 9 to about 160, about 10 to about 155, about 11 to about 150, about 12 to about 145, about 13 to about 140, about 14 to about 135, about 15 to about 130, about 16 to about 125, about 17 to about 120, about 18 to about 115, about 19 to about 110, about 20 to about 105, about 21 to about 100, about 22 to about 99, about 23 to about 98, about 24 to about 97, about 25 to about 96, about 26 to about 95, about 27 to about 94, about 28 to about 93, about 29 to about 92, about 30 to about 91, about 31 to about 90, about 32 to about 89, about 33 to about 88, about 34 to about 87, about 35 to about 86, about 36 to about 85, about 37 to about 84, about 38 to about 83, about 39 to about 82, about 40 to about 81, about 41 to about 80, about 42 to about 79, about 43 to about 78, about 44 to about 77, about 45 to about 76, about 46 to about 75, about 47 to about 74, about 48 to about 73, about 49 to about 72, about 50 to about 71, about 51 to about 70, about 52 to about 69, about 53 to about 68, about 54 to about 67, about 55 to about 66, about 56 to about 65, about 57 to about 64, about 58 to about 63, about 59 to about 62, or about 60 to about 61 nucleotides in length.

The first probe may comprise one or more binding sites. The one or more binding sites of the first probe may bind to one or more other probes, one or more analytes, one or more feature of the sample, or a combination there. In some cases, the first probe may comprise at least about 1, at least about 2, at least about 3, at least about 4, at least about 5, at least about 6, at least about 7, at least about 8, at least about 9, at least about 10, at least about 15, at least about 20, at least about 25, at least about 30, at least about 40, at least about 50, at least about 60, at least about 70, at least about 80, at least about 90, at least about 100, or more binding sites. In some cases, the first probe may comprise at most about 1, at most about 2, at most about 3, at most about 4, at most about 5, at most about 6, at most about 7, at most about 8, at most about 9, at most about 10, at most about 15, at most about 20, at most about 25, at most about 30, at most about 40, at most about 50, at most about 60, at most about 70, at most about 80, at most about 90, at most about 100, or less binding sites. In some cases, the first probe may comprise about 1 to about 100, about 2 to about 90, about 3 to about 80, about 4 to about 70, about 5 to about 60, about 6 to about 50, about 7 to about 40, about 8 to about 30, about 9 to about 25, or about 10 to about 20 binding sites.

The first probe may recognize, bind to, and/or couple to at least a portion of the first analyte. The first analyte may comprise a variety of analyte types and depending on the analyte type, the first probe may recognize, bind to, and/or couple to a specific feature. The first probe may recognize a ribonucleic acid. The first analyte may comprise the ribonucleic acid. The ribonucleic acid (RNA) of the first analyte may comprise a variety of types of RNA, including but not limited to messenger RNA (mRNA), ribosomal RNA (rRNA), transfer RNA (tRNA), micro RNA (miRNA), or a combination thereof. The RNA may be endogenous to the sample or exogenous to the sample. The RNA may have been introduced to the sample using variety of means, including but not limited to use of an AAV or rAAV. In cases where the second analyte comprises an RNA, the RNA may comprise a modification. The modification of the RNA of the first analyte may be an N 6-methyladenosine, a 5-methylcytosine, an N 1-methyladenosine, an N 7-methylguanosine, an N 4-acetylcytosine, a pseudouridine, an N1-methylpseudouridine, or a combination thereof. The RNA of the first analyte may comprise single-stranded regions, double-stranded regions, or a combination thereof. The RNA may comprise a plurality of nucleotides. The plurality of nucleotides may comprise A, C, U, G, or a combination thereof. In some cases, the RNA may comprise a secondary structure, a tertiary structure, or a combination thereof.

The first probe may recognize the first analyte. In cases where the first probe may recognize the first analyte, the first analyte may comprise a nucleic acid and the nucleic acid may comprise one or more single nucleotide polymorphism. The first probe may bind to the nucleic acid comprising the one or more single nucleotide polymorphisms. The binding of the first probe to the nucleic acid comprising the one or more single nucleotide polymorphisms may enable ligation of one end of the first probe to another end of the first probe or another end of another probe. In some cases, the ligation of the first probe may comprise ligation of one end of the first probe to another end of the first probe. In some cases, the ligation of the first probe may comprise ligation of one end of the first probe to one end of the third probe.

The first probe may not recognize an analyte. In some cases where first probe recognizes an analyte, the analyte may comprise a nucleic acid and the nucleic acid may comprise one or more single nucleotide polymorphism. The first probe may not bind to the nucleic acid comprising the one or more single nucleotide polymorphisms because the sequence of the one or more single nucleotide polymorphisms is different than what the first probe recognizes. The first probe may not be ligated in the presence of the analyte described herein. In some cases the first probe may not recognize an analyte comprising a nucleic acid comprising one or more single nucleotide polymorphisms and the first probe is not ligated if the analyte comprises the one or more single nucleotide polymorphism. The analyte may be the first analyte.

The first probe may recognize the first analyte. In some cases where the first probe may recognize the first analyte, the first analyte may comprise a nucleic acid and the nucleic acid may comprise one or more modifications. The one or more modifications of the first analyte may comprise an N 6-methyladenosine, a 5-methylcytosine, a N 1-methyladenosine, a N 7-methylguanosine, a N 4-acetylcytosine, a pseudouridine, a N1-methylpseudouridine, or a combination thereof. The first probe may bind to the nucleic acid comprising the one or more modifications. The binding of the first probe to the nucleic acid of the first analyte comprising the one or more modifications may enable ligation of one end of the first probe to another end of the first probe and/or another end of another probe. In some cases, the ligation of the first probe may comprise ligation of one end of the first probe to another end of the first probe. In some cases, the ligation of the first probe may comprise ligation of one end of the first probe to one end of the third probe.

The first probe may not recognize an analyte. In some cases where the first probe may not recognize an analyte, the analyte may comprise a nucleic acid and the nucleic acid may comprise one or more modifications. The first probe may not bind to the nucleic acid comprising the one or more modifications because the sequence of the one or more modifications is different than what the first probe recognizes. The first probe may not be ligated in the presence of the analyte described herein. In some cases, the first probe may not recognize an analyte comprising a nucleic acid comprising one or more modifications and the first probe is not ligated if the analyte comprises the one or more modifications. The analyte may be the first analyte.

The second probe may comprise a variety of components. In some cases, the second probe may comprise a nucleic acid. The nucleic acid of the second probe may comprise one or more deoxyribonucleic acids (DNA), ribonucleic acids (RNA), or a combination thereof. The nucleic acid of the second probe may comprise an oligonucleotide. The oligonucleotide of the nucleic acid of the second probe may comprise one or more modifications. The nucleic acid of the second probe may comprise one or more modifications. In some cases, the one or more modifications of the nucleic acid of the second probe may be a DNA modification. In some cases, the one or more modifications of the nucleic acid of the second probe may be an RNA modification. The one or more modifications of the nucleic acid of the second probe may comprise a phosphate modification. In some cases, the phosphate modification may comprise a 5′ phosphate modification. In some cases, the phosphate modification of the nucleic acid of the second probe may comprise a 3′ phosphate modification. In some cases, the oligonucleotide of the nucleic acid of the second probe may comprise a 5′ phosphate modification. The one or more modifications of the nucleic acid of the second probe may comprise an internucleotide linkage. The internucleotide linkage of the nucleic acid of the second probe may comprise a phosphorothioate, a phosphodiester, or a combination thereof. The internucleotide linkage may comprise a locked nucleic acid (LNA). The internucleotide of the nucleic acid of the second probe linkage may confer advantages to the methods described herein by increasing the melting temperature of oligonucleotide interactions, increasing stability of probe interactions, improving specificity of probe precognition, or a combination thereof.

The second probe may comprise a variety of lengths. In cases where the first probe comprises a nucleic acid, the nucleic acid may be at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 51, at least about 52, at least about 53, at least about 54, at least about 55, at least about 56, at least about 57, at least about 58, at least about 59, at least about 60, at least about 61, at least about 62, at least about 63, at least about 64, at least about 65, at least about 66, at least about 67, at least about 68, at least about 69, at least about 70, at least about 71, at least about 72, at least about 73, at least about 74, at least about 75, at least about 76, at least about 77, at least about 78, at least about 79, at least about 80, at least about 81, at least about 82, at least about 83, at least about 84, at least about 85, at least about 86, at least about 87, at least about 88, at least about 89, at least about 90, at least about 91, at least about 92, at least about 93, at least about 94, at least about 95, at least about 96, at least about 97, at least about 98, at least about 99, at least about 100, at least about 105, at least about 110, at least about 115, at least about 120, at least about 125, at least about 130, at least about 135, at least about 140, at least about 145, at least about 150, at least about 155, at least about 160, at least about 165, at least about 170, at least about 175, at least about 180, at least about 185, at least about 190, at least about 195, at least about 200, or more nucleotides in length. In cases where the second probe comprises a nucleic acid, the nucleic acid may be at most about 1, at most about 2, at most about 3, at most about 4, at most about 5, at most about 6, at most about 7, at most about 8, at most about 9, at most about 10, at most about 11, at most about 12, at most about 13, at most about 14, at most about 15, at most about 16, at most about 17, at most about 18, at most about 19, at most about 20, at most about 21, at most about 22, at most about 23, at most about 24, at most about 25, at most about 26, at most about 27, at most about 28, at most about 29, at most about 30, at most about 31, at most about 32, at most about 33, at most about 34, at most about 35, at most about 36, at most about 37, at most about 38, at most about 39, at most about 40, at most about 41, at most about 42, at most about 43, at most about 44, at most about 45, at most about 46, at most about 47, at most about 48, at most about 49, at most about 50, at most about 51, at most about 52, at most about 53, at most about 54, at most about 55, at most about 56, at most about 57, at most about 58, at most about 59, at most about 60, at most about 61, at most about 62, at most about 63, at most about 64, at most about 65, at most about 66, at most about 67, at most about 68, at most about 69, at most about 70, at most about 71, at most about 72, at most about 73, at most about 74, at most about 75, at most about 76, at most about 77, at most about 78, at most about 79, at most about 80, at most about 81, at most about 82, at most about 83, at most about 84, at most about 85, at most about 86, at most about 87, at most about 88, at most about 89, at most about 90, at most about 91, at most about 92, at most about 93, at most about 94, at most about 95, at most about 96, at most about 97, at most about 98, at most about 99, at most about 100, at most about 105, at most about 110, at most about 115, at most about 120, at most about 125, at most about 130, at most about 135, at most about 140, at most about 145, at most about 150, at most about 155, at most about 160, at most about 165, at most about 170, at most about 175, at most about 180, at most about 185, at most about 190, at most about 195, at most about 200, or less nucleotides in length. In cases where the second probe comprises a nucleic acid, the nucleic acid may be about 1 to about 200, about 2 to about 195, about 3 to about 190, about 4 to about 185, about 5 to about 180, about 6 to about 175, about 7 to about 170, about 8 to about 165, about 9 to about 160, about 10 to about 155, about 11 to about 150, about 12 to about 145, about 13 to about 140, about 14 to about 135, about 15 to about 130, about 16 to about 125, about 17 to about 120, about 18 to about 115, about 19 to about 110, about 20 to about 105, about 21 to about 100, about 22 to about 99, about 23 to about 98, about 24 to about 97, about 25 to about 96, about 26 to about 95, about 27 to about 94, about 28 to about 93, about 29 to about 92, about 30 to about 91, about 31 to about 90, about 32 to about 89, about 33 to about 88, about 34 to about 87, about 35 to about 86, about 36 to about 85, about 37 to about 84, about 38 to about 83, about 39 to about 82, about 40 to about 81, about 41 to about 80, about 42 to about 79, about 43 to about 78, about 44 to about 77, about 45 to about 76, about 46 to about 75, about 47 to about 74, about 48 to about 73, about 49 to about 72, about 50 to about 71, about 51 to about 70, about 52 to about 69, about 53 to about 68, about 54 to about 67, about 55 to about 66, about 56 to about 65, about 57 to about 64, about 58 to about 63, about 59 to about 62, or about 60 to about 61 nucleotides in length.

The second probe may comprise one or more binding sites. The one or more binding sites may bind to one or more other probes, one or more analytes, one or more feature of the sample, or a combination there. In some cases, the second probe may comprise at least about 1, at least about 2, at least about 3, at least about 4, at least about 5, at least about 6, at least about 7, at least about 8, at least about 9, at least about 10, at least about 15, at least about 20, at least about 25, at least about 30, at least about 40, at least about 50, at least about 60, at least about 70, at least about 80, at least about 90, at least about 100, or more binding sites. In some cases, the second probe may comprise at most about 1, at most about 2, at most about 3, at most about 4, at most about 5, at most about 6, at most about 7, at most about 8, at most about 9, at most about 10, at most about 15, at most about 20, at most about 25, at most about 30, at most about 40, at most about 50, at most about 60, at most about 70, at most about 80, at most about 90, at most about 100, or less binding sites. In some cases, the second probe may comprise about 1 to about 100, about 2 to about 90, about 3 to about 80, about 4 to about 70, about 5 to about 60, about 6 to about 50, about 7 to about 40, about 8 to about 30, about 9 to about 25, or about 10 to about 20 binding sites.

The second probe may comprise an aptamer. The aptamer of the second probe may comprise a nucleic acid. The aptamer of the second probe may comprise a secondary structure, a tertiary structure or a combination thereof. The aptamer may be configured to recognize, bind to, and/or couple to an analyte. For example, the aptamer may comprise a sequence and associated tertiary structure that recognizes and binds to a protein or interest. The analyte recognized by, bound to, and/or coupled to by the aptamer may be the second analyte. The analyte recognized by, bound to, and/or coupled to by the aptamer may comprise a nucleic acid, a polypeptide, or a combination thereof. For example, the analyte may comprise a DNA-binding protein comprising an epitope recognized by the aptamer. The aptamer may comprise RNA, DNA, a polypeptide, xeno nucleic acid (XNA), or a combination thereof. The aptamer may have a molecular weight of at least about 1 kilodaltons (kDa), at least about 2 kDa, at least about 3 kDa, at least about 4 kDa, at least about 5 kDa, at least about 6 kDa, at least about 7 kDa, at least about 8 kDa, at least about 9 kDa, at least about 10 kDa, at least about 11 kDa, at least about 12 kDa, at least about 13 kDa, at least about 14 kDa, at least about 15 kDa, at least about 16 kDa, at least about 17 kDa, at least about 18 kDa, at least about 19 kDa, at least about 20 kDa, at least about 21 kDa, at least about 22 kDa, at least about 23 kDa, at least about 24 kDa, at least about 25 kDa, at least about 26 kDa, at least about 27 kDa, at least about 28 kDa, at least about 29 kDa, at least about 30 kDa, at least about 31 kDa, at least about 32 kDa, at least about 33 kDa, at least about 34 kDa, at least about 35 kDa, at least about 36 kDa, at least about 37 kDa, at least about 38 kDa, at least about 39 kDa, at least about 40 kDa, at least about 41 kDa, at least about 42 kDa, at least about 43 kDa, at least about 44 kDa, at least about 45 kDa, at least about 46 kDa, at least about 47 kDa, at least about 48 kDa, at least about 49 kDa, or at least about 50 kDa. The aptamer may have a molecular weight of at most about 1 kDa, at most about 2 kDa, at most about 3 kDa, at most about 4 kDa, at most about 5 kDa, at most about 6 kDa, at most about 7 kDa, at most about 8 kDa, at most about 9 kDa, at most about 10 kDa, at most about 11 kDa, at most about 12 kDa, at most about 13 kDa, at most about 14 kDa, at most about 15 kDa, at most about 16 kDa, at most about 17 kDa, at most about 18 kDa, at most about 19 kDa, at most about 20 kDa, at most about 21 kDa, at most about 22 kDa, at most about 23 kDa, at most about 24 kDa, at most about 25 kDa, at most about 26 kDa, at most about 27 kDa, at most about 28 kDa, at most about 29 kDa, at most about 30 kDa, at most about 31 kDa, at most about 32 kDa, at most about 33 kDa, at most about 34 kDa, at most about 35 kDa, at most about 36 kDa, at most about 37 kDa, at most about 38 kDa, at most about 39 kDa, at most about 40 kDa, at most about 41 kDa, at most about 42 kDa, at most about 43 kDa, at most about 44 kDa, at most about 45 kDa, at most about 46 kDa, at most about 47 kDa, at most about 48 kDa, at most about 49 kDa, or at most about 50 kDa. The aptamer may have a molecular weight of about 1 to about 50 kDa, about 2 to about 49 kDa, about 3 to about 48 kDa, about 4 to about 47 kDa, about 5 to about 46 kDa, about 6 to about 45 kDa, about 7 to about 44 kDa, about 8 to about 43 kDa, about 9 to about 42 kDa, about 10 to about 41 kDa, about 11 to about 40 kDa, about 12 to about 39 kDa, about 13 to about 38 kDa, about 14 to about 37 kDa, about 15 to about 36 kDa, about 16 to about 35 kDa, about 17 to about 34 kDa, about 18 to about 33 kDa, about 19 to about 32 kDa, about 20 to about 31 kDa, about 21 to about 30 kDa, about 22 to about 29 kDa, about 23 to about 28 kDa, or about 24 to about 27 kDa, about 25 to about 26 kDa.

The second probe may comprise a polypeptide. The polypeptide of the second probe may comprise a protein, a peptide or a combination thereof. The protein of the polypeptide of the second probe comprises a be a protein-binding protein, a DNA-binding protein, or a combination thereof. The protein of the polypeptide of the second probe may be a nucleic acid binding protein. The protein may comprise an antibody, antibody fragment, affimer, nanobody, or a combination thereof. The antibody or antibody fragment of the second probe may comprise a variety of isotypes including but not limited to IgG, IgM, IgA, IgD, IgE, or a combination thereof. The antibody or antibody fragment of the second probe may comprise an fc domain that recognizes an analyte (e.g. the second analyte). The second probe may comprise one or more antibodies or antibody fragments. In some cases, the second probe may comprise an antibody that recognizes, binds to, and/or couples to the second analyte and an antibody that recognizes, binds to, and/or couples to the antibody that that recognizes, binds to, and/or couples to the second analyte. One or more of the antibodies of the second probe may comprise one or more modifications. The one or more modifications of the one or more antibodies of the second probe may comprise a nucleic acid modification conjugated to one or more of the antibodies or antibody fragments of the second probe either directly or indirectly. The nucleic acid modification of the second probe may comprise a binding site (e.g. the fourth binding site of the second probe). The binding site of the nucleic acid modification of the second probe may serve as a primer to initiate amplification. The binding site of the nucleic acid modification of the second probe may serve as a binding site for one or more portions of the first probe and/or third probe, and upon binding of the one or more portions of the first probe, may initiate ligation of one end of the first probe to another end of the first probe and/or another end of the third probe.

The second probe may recognize the second analyte. The second analyte may comprise a variety of analyte types and depending on the analyte type, the second probe may recognize a specific feature. For example, the second probe may recognize and bind to a ribosomal protein and a specific feature of the ribosomal protein may comprise an epitope that is recognized and bound by an anti-ribosomal antibody. The second analyte may comprise the ribonucleic acid. The ribonucleic acid (RNA) of the second analyte may comprise a variety of types of RNA, including but not limited to messenger RNA (mRNA), ribosomal RNA (rRNA), transfer RNA (tRNA), micro RNA (miRNA), or a combination thereof. The RNA may be endogenous to the sample or exogenous to the sample. The RNA of the nucleic acid of the second analyte may have been introduced to the sample using variety of means, including but not limited to use of an AAV or rAAV. In cases where the second analyte comprises an RNA, the RNA may comprise a modification. The modification may be an N 6-methyladenosine, a 5-methylcytosine, an N 1-methyladenosine, an N 7-methylguanosine, an N 4-acetylcytosine, a pseudouridine, an N1-methylpseudouridine, or a combination thereof. The RNA of the nucleic acid of the second analyte may comprise single-stranded regions, double-stranded regions, or a combination thereof. The RNA may comprise a plurality of nucleotides. The plurality of nucleotides may comprise A, C, U, G, or a combination thereof. In some cases, the RNA may comprise a secondary structure, a tertiary structure, or a combination thereof.

The second probe may recognize the second analyte. In some cases where the second probe may recognize the second analyte, the second analyte may comprise a nucleic acid and the nucleic acid may comprise one or more modifications. The nucleic acid of the second analyte may comprise an RNA. The one or more modifications of the nucleic acid of the second analyte may comprise an N 6-methyladenosine, a 5-methylcytosine, a N 1-methyladenosine, a N 7-methylguanosine, a N 4-acetylcytosine, a pseudouridine, a N1-methylpseudouridine, or a combination thereof. The second probe may bind to the nucleic acid comprising the one or more modifications.

The second probe may recognize a polypeptide. The polypeptide of the second analyte may comprise a protein, a peptide or a combination thereof. In cases where the polypeptide of the second analyte comprises a protein, the protein may comprise a transcription factor, a ribosomal protein, a histone, a polymerase, a helicase, a restriction enzyme, a ribonucleic acid binding protein, an enzyme, an antibody, a structural protein, a defense protein, a signaling protein, a receptor, a soluble protein, a transmembrane or a combination thereof. In cases where the protein may be a signaling protein, the signaling protein may be a cytokine, chemokine, or a combination thereof. In cases where the polypeptide of the second analyte comprises a protein, the protein may comprise a transcription factor, a ribosomal protein, a histone, a polymerase, or a combination thereof. In cases where the protein of the second analyte comprises a ribosomal protein, the ribosomal protein may comprise an S3A ribosomal protein, an SA ribosomal protein, an S3 ribosomal protein, an S9 ribosomal protein, an S4 (X, Y1, Y2) ribosomal protein, an S2 ribosomal protein, an S6 ribosomal protein, an S5 ribosomal protein, an S7 ribosomal protein, an S15A ribosomal protein, an S8 ribosomal protein, an S16 ribosomal protein, an S20 ribosomal protein, an S10 ribosomal protein, an S14 ribosomal protein, an S23 ribosomal protein, an S12 ribosomal protein, an S18 ribosomal protein, an S29 ribosomal protein, an S13 ribosomal protein, an S11 ribosomal protein, an S17 ribosomal protein, an S15 ribosomal protein, an S19 ribosomal protein, an S21 ribosomal protein, an S24 ribosomal protein, an S25 ribosomal protein, an S26 ribosomal protein, an S27 ribosomal protein, an S28 ribosomal protein, an S30 ribosomal protein, an S27A ribosomal protein, an RACK1 ribosomal protein, an L10A ribosomal protein, an L8 ribosomal protein, an L3 ribosomal protein, an L4 ribosomal protein, an L11 ribosomal protein, an L9 ribosomal protein, an L6 ribosomal protein, an L7A ribosomal protein, an P0 ribosomal protein, an L12 ribosomal protein, an L13A ribosomal protein, an L13 ribosomal protein, an L23 ribosomal protein, an L14 ribosomal protein, an L27A ribosomal protein, an L15 ribosomal protein, an L10 ribosomal protein, an L5 ribosomal protein, an L18 ribosomal protein, an L19 ribosomal protein, an L18A ribosomal protein, an L21 ribosomal protein, an L17 ribosomal protein, an L22 ribosomal protein, an L23A ribosomal protein, an L26 ribosomal protein, an L24 ribosomal protein, an L27 ribosomal protein, an L28 ribosomal protein, an L35 ribosomal protein, an L29 ribosomal protein, an L7 ribosomal protein, an L30 ribosomal protein, an L31 ribosomal protein, an L32 ribosomal protein, an L35A ribosomal protein, an L34 ribosomal protein, an L36 ribosomal protein, an L37 ribosomal protein, an L38 ribosomal protein, an L39 ribosomal protein, an L40 ribosomal protein, an L41 ribosomal protein, an L36A ribosomal protein, an L37A ribosomal protein, a P1/P2 (αβ) ribosomal protein, or a combination thereof.

The second probe may recognize one or more post-translational modification of a protein. The one or more post translational modifications of the protein of the second analyte may comprise a myristoylation, a palmitoylation, a farnesylation, a geranylgeranylation, a glypiation, a glycosylphosphatidylinositol, a lipoylation, a flavin moiety attachment, a heme C attachment, a phosphopantetheinylation, a retinylidene Schiff base formation, a modifications of translation factors, a diphthamide formation, a ethanolamine phosphoglycerol, a hypusine formation, a beta-Lysine addition on a lysine, a acylation (e.g. O-acylation, N-acylation, and S-acylation), an acetylation, a formylation, a alkylation, a amidation, a arginylation, a polyglutamylation, a polyglycylation, a butyrylation, a gamma-carboxylation, a glycosylation, a polysialylation, a malonylation, a hydroxylation, a nucleotide addition, a phosphate ester (O-linked), a phosphoramidate (N-linked) formation, a phosphorylation, a adenylylation, a uridylylation, a propionylation, a pyroglutamate formation, a S-glutathionylation, a S-nitrosylation, a S-sulfenylation, a S-sulfinylation, a S-sulfonylation, a succinylation, a sulfation, a glycation, a carbamylation, a carbonylation, a spontaneous isopeptide bond formation, a biotinylation, a carbamylation, an oxidation, a pegylation, an ubiquitination, a SUMOylation, a neddylation, an ISGylation, a citrullination, a deamidation, an eliminylation, or a combination thereof. The second analyte may comprise a chemical modification. The one or more post translational modifications of the protein of the second analyte may comprise an alkylation, a phosphorylation, or a combination thereof.

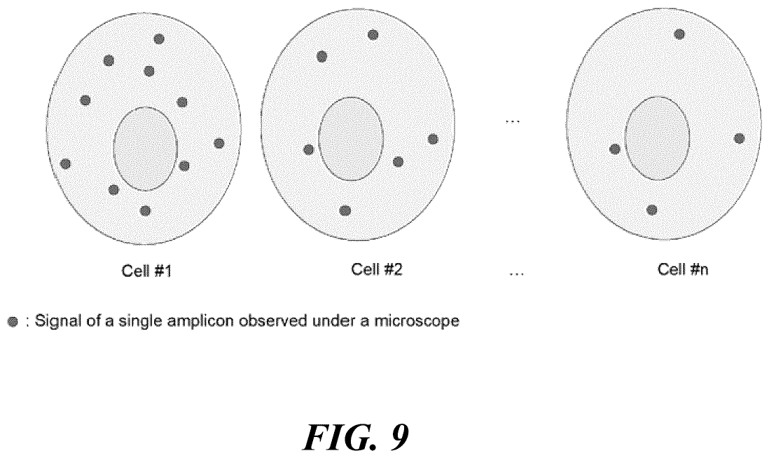

The methods described herein also relate to analyzing the proximity of one or more analytes using one or more barcodes or derivatives thereof. The barcode or derivative thereof may confer information regarding one or more analytes, the proximity of one or more analytes, or a combination thereof. The barcode or derivative thereof may indicate with the presence of an analyte. For example, detection of the barcode or derivative thereof within a tissue sample may indicate that an analyte that was recognized and bound to one or more probes comprising the barcode is present within the sample. The barcode or derivative thereof of may indicate the proximity of one analyte to another analyte. For example, detection of the barcode or derivative thereof within a tissue sample may indicate that a first analyte and a second analyte are complexed to each other within the tissue sample. The barcode or derivative thereof may be used in combination with other information to indicate the presence of an analyte and/or spatial localization of the analyte. For example, the barcode or derivative thereof may be detected as a result of the first and second probe binding to a messenger RNA sequence comprising a specific single nucleotide polymorphism. In combination, the sample may be stained with a nuclear stain, which may be detected using an imaging system. Detection of the barcode or derivative thereof and detection of the nuclear stain may provide information related to the presence of the messenger RNA comprising the single nucleotide polymorphism and its location within and/or outside of nuclei within the sample. The barcode or derivative thereof may be used in combination with other information to indicate the proximity of one analyte to another analyte. For example, the barcode or derivative thereof may be detected to determine whether a specific messenger RNA sequence is in close proximity of a ribosomal RNA and a dye-labeled antibody may be used to detect the presence of a ribosomal protein. In combination, the detection of the barcode or derivative thereof and detection of the dye-labeled antibody may provide information related to the translation status of the messenger RNA. The barcode or derivative thereof may be detected in situ. The barcode or derivative thereof may be detected using one or more rounds of detection. For example, the barcode or derivative thereof may comprise 8 nucleotides, and sequence information associated with 2 nucleotides of the 8 nucleotides may be acquired across 7 rounds of detection using detection and anchor probes and removal of the corresponding detection and anchor probes. FIG. 9 shows how data related to the barcode sequence may be collected across multiple rounds. The amplicon can be quantified under a fluorescence microscope using in situ sequencing. The number of amplicons or fluorescence signal intensities of the amplicons represent the translation efficiency of the specific mRNA in the specific cells. The amplicon from different mRNA species can be distinguished by different Barcode A sequences.

The probes described herein also, including the first probe, the second probe, the third probe and other probes, may each comprise one or more barcodes. In some cases a probe of the methods described herein may comprise at least about 1 barcode, at least about 2 barcodes, at least about 3 barcodes, at least about 4 barcodes, at least about 5 barcodes, at least about 6 barcodes, at least about 7 barcodes, at least about 8 barcodes, at least about 9 barcodes, at least about 10 barcodes, at least about 11 barcodes, at least about 12 barcodes, at least about 13 barcodes, at least about 14 barcodes, at least about 15 barcodes, at least about 16 barcodes, at least about 17 barcodes, at least about 18 barcodes, at least about 19 barcodes, at least about 20 barcodes, at least about 21 barcodes, at least about 22 barcodes, at least about 23 barcodes, at least about 24 barcodes, at least about 25 barcodes, at least about 26 barcodes, at least about 27 barcodes, at least about 28 barcodes, at least about 29 barcodes, at least about 30 barcodes, at least about 31 barcodes, at least about 32 barcodes, at least about 33 barcodes, at least about 34 barcodes, at least about 35 barcodes, at least about 36 barcodes, at least about 37 barcodes, at least about 38 barcodes, at least about 39 barcodes, at least about 40 barcodes, at least about 41 barcodes, at least about 42 barcodes, at least about 43 barcodes, at least about 44 barcodes, at least about 45 barcodes, at least about 46 barcodes, at least about 47 barcodes, at least about 48 barcodes, at least about 49 barcodes, at least about 50, or more barcodes. In some cases a probe of the methods described herein may comprise at most about 1 barcode, at most about 2 barcodes, at most about 3 barcodes, at most about 4 barcodes, at most about 5 barcodes, at most about 6 barcodes, at most about 7 barcodes, at most about 8 barcodes, at most about 9 barcodes, at most about 10 barcodes, at most about 11 barcodes, at most about 12 barcodes, at most about 13 barcodes, at most about 14 barcodes, at most about 15 barcodes, at most about 16 barcodes, at most about 17 barcodes, at most about 18 barcodes, at most about 19 barcodes, at most about 20 barcodes, at most about 21 barcodes, at most about 22 barcodes, at most about 23 barcodes, at most about 24 barcodes, at most about 25 barcodes, at most about 26 barcodes, at most about 27 barcodes, at most about 28 barcodes, at most about 29 barcodes, at most about 30 barcodes, at most about 31 barcodes, at most about 32 barcodes, at most about 33 barcodes, at most about 34 barcodes, at most about 35 barcodes, at most about 36 barcodes, at most about 37 barcodes, at most about 38 barcodes, at most about 39 barcodes, at most about 40 barcodes, at most about 41 barcodes, at most about 42 barcodes, at most about 43 barcodes, at most about 44 barcodes, at most about 45 barcodes, at most about 46 barcodes, at most about 47 barcodes, at most about 48 barcodes, at most about 49 barcodes, at most about 50, or more barcodes. In some cases a probe of the methods described herein may comprise about 1 to about 50 barcodes, about 2 to about 49 barcodes, about 3 to about 48 barcodes, about 4 to about 47 barcodes, about 5 to about 46 barcodes, about 6 to about 45 barcodes, about 7 to about 44 barcodes, about 8 to about 43 barcodes, about 9 to about 42 barcodes, about 10 to about 41 barcodes, about 11 to about 40 barcodes, about 12 to about 39 barcodes, about 13 to about 38 barcodes, about 14 to about 37 barcodes, about 15 to about 36 barcodes, about 16 to about 35 barcodes, about 17 to about 34 barcodes, about 18 to about 33 barcodes, about 19 to about 32 barcodes, about 20 to about 31 barcodes, about 21 to about 30 barcodes, about 22 to about 29 barcodes, about 23 to about 28 barcodes, or about 24 to about 27 barcodes, or about 25 to about 26 barcodes. The first probe may comprise one barcode, two barcodes, three barcodes, four barcodes, or more. The first probe may comprise two barcodes, wherein the second barcode of the two barcodes corresponds to the first analyte. The first probe may comprise two barcodes. The second barcode of the two barcodes corresponds to the second analyte. The first probe may comprise two barcodes. The second barcode of the two barcodes corresponds to the first analyte being proximal to the second analyte.

A barcode of the one or more barcodes described herein may comprise a nucleic acid. The nucleic acid may comprise a DNA, an RNA, or a combination thereof. The nucleic acid may comprise a plurality of nucleotides including but not limited to A, T, C, U, G, or a combination thereof. The barcode nucleic acid may comprise one or more non-natural nucleotides. The nucleic acid may comprise one or more nucleic acid modifications. The nucleic acid may comprise a variety of lengths, including but not limited to at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 51, at least about 52, at least about 53, at least about 54, at least about 55, at least about 56, at least about 57, at least about 58, at least about 59, at least about 60, at least about 61, at least about 62, at least about 63, at least about 64, at least about 65, at least about 66, at least about 67, at least about 68, at least about 69, at least about 70, at least about 71, at least about 72, at least about 73, at least about 74, at least about 75, at least about 76, at least about 77, at least about 78, at least about 79, at least about 80, at least about 81, at least about 82, at least about 83, at least about 84, at least about 85, at least about 86, at least about 87, at least about 88, at least about 89, at least about 90, at least about 91, at least about 92, at least about 93, at least about 94, at least about 95, at least about 96, at least about 97, at least about 98, at least about 99, at least about 100, at least about 105, at least about 110, at least about 115, at least about 120, at least about 125, at least about 130, at least about 135, at least about 140, at least about 145, at least about 150, at least about 155, at least about 160, at least about 165, at least about 170, at least about 175, at least about 180, at least about 185, at least about 190, at least about 195, at least about 200, or more nucleotides in length. The nucleic acid may have a length of at most about 1, at most about 2, at most about 3, at most about 4, at most about 5, at most about 6, at most about 7, at most about 8, at most about 9, at most about 10, at most about 11, at most about 12, at most about 13, at most about 14, at most about 15, at most about 16, at most about 17, at most about 18, at most about 19, at most about 20, at most about 21, at most about 22, at most about 23, at most about 24, at most about 25, at most about 26, at most about 27, at most about 28, at most about 29, at most about 30, at most about 31, at most about 32, at most about 33, at most about 34, at most about 35, at most about 36, at most about 37, at most about 38, at most about 39, at most about 40, at most about 41, at most about 42, at most about 43, at most about 44, at most about 45, at most about 46, at most about 47, at most about 48, at most about 49, at most about 50, at most about 51, at most about 52, at most about 53, at most about 54, at most about 55, at most about 56, at most about 57, at most about 58, at most about 59, at most about 60, at most about 61, at most about 62, at most about 63, at most about 64, at most about 65, at most about 66, at most about 67, at most about 68, at most about 69, at most about 70, at most about 71, at most about 72, at most about 73, at most about 74, at most about 75, at most about 76, at most about 77, at most about 78, at most about 79, at most about 80, at most about 81, at most about 82, at most about 83, at most about 84, at most about 85, at most about 86, at most about 87, at most about 88, at most about 89, at most about 90, at most about 91, at most about 92, at most about 93, at most about 94, at most about 95, at most about 96, at most about 97, at most about 98, at most about 99, at most about 100, at most about 105, at most about 110, at most about 115, at most about 120, at most about 125, at most about 130, at most about 135, at most about 140, at most about 145, at most about 150, at most about 155, at most about 160, at most about 165, at most about 170, at most about 175, at most about 180, at most about 185, at most about 190, at most about 195, at most about 200, or fewer nucleotides in length. The nucleic acid may have a length of at most about 1 to about 200, about 2 to about 195, about 3 to about 190, about 4 to about 185, about 5 to about 180, about 6 to about 175, about 7 to about 170, about 8 to about 165, about 9 to about 160, about 10 to about 155, about 11 to about 150, about 12 to about 145, about 13 to about 140, about 14 to about 135, about 15 to about 130, about 16 to about 125, about 17 to about 120, about 18 to about 115, about 19 to about 110, about 20 to about 105, about 21 to about 100, about 22 to about 99, about 23 to about 98, about 24 to about 97, about 25 to about 96, about 26 to about 95, about 27 to about 94, about 28 to about 93, about 29 to about 92, about 30 to about 91, about 31 to about 90, about 32 to about 89, about 33 to about 88, about 34 to about 87, about 35 to about 86, about 36 to about 85, about 37 to about 84, about 38 to about 83, about 39 to about 82, about 40 to about 81, about 41 to about 80, about 42 to about 79, about 43 to about 78, about 44 to about 77, about 45 to about 76, about 46 to about 75, about 47 to about 74, about 48 to about 73, about 49 to about 72, about 50 to about 71, about 51 to about 70, about 52 to about 69, about 53 to about 68, about 54 to about 67, about 55 to about 66, about 56 to about 65, about 57 to about 64, about 58 to about 63, about 59 to about 62, or about 60 to about 61 nucleotides in length.

A barcode of the one or more barcodes described herein also may correspond to the first analyte. For example, detection of the barcode or derivative thereof in a sample may indicate the presence of the first analyte. A barcode of the one or more barcodes described herein also may correspond to the second analyte. For example, detection of the barcode or derivative thereof in a sample may indicate the presence of the second analyte. A barcode of the one or more barcodes described herein also may correspond to one analyte being proximal to another analyte. For example, detection of the barcode or derivative thereof in a sample may indicate that a first analyte is complexed, or otherwise in close proximity to, a second analyte within the sample. In some cases, the barcode may correspond to the first analyte being proximal to the second analyte. The barcode or a derivative of the barcode may be detected to correspond to the proximity of the first analyte to the second analyte.

A derivative of the barcode may comprise a reverse complement of the barcode. The reverse complement of the barcode may comprise a sequence that is capable of hybridizing to the barcode. The derivative of the barcode may be formed from an amplification reaction. The amplification reaction may comprise a rolling circle amplification reaction where an amplicon is generated and comprises multiple copies of the derivative of the barcode. The multiple copies of the derivative of the barcode may be connected to each other within the amplicon.

The methods described herein also may comprise amplifying a circular oligonucleotide by performing an amplification reaction. The circular oligonucleotide may be formed by ligating two ends of the first probe. The circular oligonucleotide may be formed by ligating one end of the first probe to one end of the third probe and ligating a second end of the first probe to a second end of the third probe. The amplification reaction may comprise performing a rolling circle amplification reaction using the circular oligonucleotide. The amplification reaction may comprise use of a primer that binds to the circular oligonucleotide and initiates amplification. The primer that binds to the circular oligonucleotide may comprise a portion of one or more probes described herein. For example, the second probe may bind to the circular oligonucleotide and initiate a rolling circle amplification reaction. The rolling circle amplification reaction may generate one or more amplicons. The one or more amplicons generated by the rolling circle amplification reaction may comprise multiple copies of the circular oligonucleotide. The multiple copies of the circular oligonucleotide may be concatenated together to form a long strand of nucleic acid. T The circular oligonucleotide may comprise one or more copies of a barcode or derivative thereof. The one or more copies of a barcode may comprise one or more copies of the same barcode (e.g. the same nucleic acid sequence). The one or more copies of a barcode may comprise one or more copies of different barcodes. Each of the one or more amplicons generated by a rolling circle amplification may comprise at least 1 copy, at least 2 copies, at least 3 copies, at least about 4 copies, at least about 5 copies, at least about 6 copies, at least about 7 copies, at least about 8 copies, at least about 9 copies, at least about 10 copies, at least about 20 copies, at least about 30 copies, at least about 40 copies, at least about 50 copies, at least about 60 copies, at least about 70 copies, at least about 80 copies, at least about 90 copies, at least about 100 copies, at least about 135 copies, at least about 150 copies, at least about 175 copies, at least about 200 copies, at least about 300 copies, at least about 400 copies, at least about 500 copies, at least about 600 copies, at least about 700 copies, at least about 800 copies, at least about 900 copies, at least about 1000 copies, at least about 2000 copies, at least about 3000 copies, at least about 4000 copies, at least about 5000 copies, at least about 6000 copies, at least about 7000 copies, at least about 8000 copies, at least about 9000 copies, at least about 10000 copies, at least about 50000 copies, at least about 100000 copies, at least about 500000 copies, at least about 1000000, or more copies of the one or more barcodes or a reverse complement of the one or more barcodes of the circular oligonucleotide. Each of the one or more amplicons generated by a rolling circle amplification may comprise at most 1 copy, at most 2 copies, at most 3 copies, at most about 4 copies, at most about 5 copies, at most about 6 copies, at most about 7 copies, at most about 8 copies, at most about 9 copies, at most about 10 copies, at most about 20 copies, at most about 30 copies, at most about 40 copies, at most about 50 copies, at most about 60 copies, at most about 70 copies, at most about 80 copies, at most about 90 copies, at most about 100 copies, at most about 135 copies, at most about 150 copies, at most about 175 copies, at most about 200 copies, at most about 300 copies, at most about 400 copies, at most about 500 copies, at most about 600 copies, at most about 700 copies, at most about 800 copies, at most about 900 copies, at most about 1000 copies, at most about 2000 copies, at most about 3000 copies, at most about 4000 copies, at most about 5000 copies, at most about 6000 copies, at most about 7000 copies, at most about 8000 copies, at most about 9000 copies, at most about 10000 copies, at most about 50000 copies, at most about 100000 copies, at most about 500000 copies, at most about 1000000, or fewer copies of the one or more barcodes or a reverse complement of the one or more barcodes of the circular oligonucleotide. Each of the one or more amplicons generated by a rolling circle amplification may comprise about 1 to about 1000000 copies, about 2 to about 500000 copies, about 3 to about 100000 copies, about 4 to about 50000 copies, about 5 to about 10000 copies, about 6 to about 9000 copies, about 7 to about 8000 copies, about 8 to about 7000 copies, about 9 to about 6000 copies, about 10 to about 5000 copies, about 20 to about 4000 copies, about 30 to about 3000 copies, about 40 to about 2000 copies, about 50 to about 1000 copies, about 60 to about 900 copies, about 70 to about 800 copies, about 80 to about 700 copies, about 90 to about 600 copies, about 100 to about 500 copies, about 135 to about 400 copies, about 150 to about 300 copies, about 175 to about 200 copies of the one or more barcodes or a reverse complement of the one or more barcodes of the circular oligonucleotide.

The rolling circle amplification reaction may be performed using a polymerase. The polymerase may be a DNA polymerase. The DNA polymerase may comprise Q5 High-Fidelity DNA Polymerase, Q5U Hot Start High-Fidelity DNA Polymerase, Phusion High-Fidelity DNA Polymerase*, Routine PCR, OneTaq DNA Polymerase, Taq DNA Polymerase, LongAmp Taq DNA Polymerase, Hemo KlenTaq, Epimark Hot Start Taq DNA Polymerase, Isothermal Amplification and Strand Displacement, Bst DNA Polymerase, Bst DNA Polymerase, Bst 2.0 DNA Polymerase, Bst 3.0 DNA Polymerase, Bsu DNA Polymerase, Large Fragment, phi29 DNA Polymerase, phi29-XT DNA Polymerase, T7 DNA Polymerase (unmodified), Sulfolobus DNA Polymerase IV, Therminator™ DNA Polymerase, DNA Polymerase I (E. coli), DNA Polymerase I, Large (Klenow) Fragment′, Klenow Fragment (3′→5′ exo-), T4 DNA Polymerase, Legacy Polymerases, Vent DNA Polymerase, Vent (exo-) DNA Polymerase, Deep Vent DNA Polymerase, Deep Vent (exo-) DNA Polymerase, or a combination thereof. The polymerase may comprise phi29 DNA Polymerase, phi29-XT DNA Polymerase, or a combination thereof.

The amplification reaction may be performed using a buffer. The buffer may comprise a variety of components including but not limited to MgCl₂, NaCl, CaCl₂, ethylenediaminetetraacetic acid (EDTA), 2-[4-(2,4,4-trimethylpentan-2-yl)phenoxy]ethanol (Triton X-100), polysorbate 20 (Tween 20), sodium lauryl sulfate (SDS), 2-Amino-2-hydroxymethyl-propane-1,3-diol (tris), sheared DNA, water, or a combination thereof.

The amplification reaction may be performed at a temperature. The temperature may be about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., or higher. The temperature may be about 4 to about 95° C., about 5 to about 94° C., about 6 to about 93° C., about 7 to about 92° C., about 8 to about 91° C., about 9 to about 90° C., about 10 to about 89° C., about 11 to about 88° C., about 12 to about 87° C., about 13 to about 86° C., about 14 to about 85° C., about 15 to about 84° C., about 16 to about 83° C., about 17 to about 82° C., about 18 to about 81° C., about 19 to about 80° C., about 20 to about 79° C., about 21 to about 78° C., about 22 to about 77° C., about 23 to about 76° C., about 24 to about 75° C., about 25 to about 74° C., about 26 to about 73° C., about 27 to about 72° C., about 28 to about 71° C., about 29 to about 70° C., about 30 to about 69° C., about 31 to about 68° C., about 32 to about 67° C., about 33 to about 66° C., about 34 to about 65° C., about 35 to about 64° C., about 36 to about 63° C., about 37 to about 62° C., about 38 to about 61° C., about 39 to about 60° C., about 40 to about 59° C., about 41 to about 58° C., about 42 to about 57° C., about 43 to about 56° C., about 44 to about 55° C., about 45 to about 54° C., about 46 to about 53° C., about 47 to about 52° C., about 48 to about 51° C., or about 49 to about 50° C.

The amplification reaction may be performed for a length of time. The length of time may be at least about 5 minutes, at least about 10 minutes, at least about 15 minutes, at least about 20 minutes, at least about 25 minutes, at least about 30 minutes, at least about 40 minutes, at least about 45 minutes, at least about 50 minutes, at least about 55 minutes, at least about 60 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 1 day, at least about 2 days, at least about 3 days at least about 4 days or longer. The length of time may be at most about 5 minutes, at most about 10 minutes, at most about 15 minutes, at most about 20 minutes, at most about 25 minutes, at most about 30 minutes, at most about 40 minutes, at most about 45 minutes, at most about 50 minutes, at most about 55 minutes, at most about 60 minutes, at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 11 hours, at most about 12 hours, at most about 13 hours, at most about 14 hours, at most about 15 hours, at most about 16 hours, at most about 17 hours, at most about 18 hours, at most about 19 hours, at most about 20 hours, at most about 21 hours, at most about 22 hours, at most about 23 hours, at most about 24 hours, at most about 1 day, at most about 2 days, at most about 3 days at most about 4 days or less. The length of time may be about 5 minutes to about 24 hours, about 10 minutes to about 23 hours, about 15 minutes to about 22 hours, about 20 minutes to about 21 hours, about 25 minutes to about 20 hours, about 30 minutes to about 19 hours, about 40 minutes to about 18 hours, about 45 minutes to about 17 hours, about 50 minutes to about 16 hours, about 55 minutes to about 15 hours, about 60 minutes to about 14 hours, about 1 hour to about 13 hours, about 2 hours to about 12 hours, about 3 hours to about 11 hours, about 4 hours to about 10 hours, about 5 hours to about 9 hours, or about 6 hours to about 8 hours.

The second probe may be a primer for the amplification reaction. The second probe may bind to the circular oligonucleotide to initiate amplification. For example, the second probe may bind (e.g. hybridize to) the circular oligonucleotide and bind the second analyte. The second probe may bind the circular oligonucleotide to form a duplex nucleic acid with an overhang region. The duplex nucleic acid formed between the second probe and the circular oligonucleotide may comprise at least about 1 base pair, at least about 2 base pairs, at least about 3 base pairs, at least about 4 base pairs, at least about 5 base pairs, at least about 6 base pairs, at least about 7 base pairs, at least about 8 base pairs, at least about 9 base pairs, at least about 10 base pairs, at least about 11 base pairs, at least about 12 base pairs, at least about 13 base pairs, at least about 14 base pairs, at least about 15 base pairs, at least about 16 base pairs, at least about 17 base pairs, at least about 18 base pairs, at least about 19 base pairs, at least about 20 base pairs, at least about 21 base pairs, at least about 22 base pairs, at least about 23 base pairs, at least about 24 base pairs, at least about 25 base pairs, at least about 26 base pairs, at least about 27 base pairs, at least about 28 base pairs, at least about 29 base pairs, at least about 30 base pairs, or more base pairs. The duplex nucleic acid may comprise at most about 1 base pair, at most about 2 base pairs, at most about 3 base pairs, at most about 4 base pairs, at most about 5 base pairs, at most about 6 base pairs, at most about 7 base pairs, at most about 8 base pairs, at most about 9 base pairs, at most about 10 base pairs, at most about 11 base pairs, at most about 12 base pairs, at most about 13 base pairs, at most about 14 base pairs, at most about 15 base pairs, at most about 16 base pairs, at most about 17 base pairs, at most about 18 base pairs, at most about 19 base pairs, at most about 20 base pairs, at most about 21 base pairs, at most about 22 base pairs, at most about 23 base pairs, at most about 24 base pairs, at most about 25 base pairs, at most about 26 base pairs, at most about 27 base pairs, at most about 28 base pairs, at most about 29 base pairs, at most about 30 base pairs, or fewer base pairs. The duplex nucleic acid may comprise about 1 to about 30 base pairs, about 2 to about 29 base pairs, about 3 to about 28 base pairs, about 4 to about 27 base pairs, about 5 to about 26 base pairs, about 6 to about 25 base pairs, about 7 to about 24 base pairs, about 8 to about 23 base pairs, about 9 to about 22 base pairs, about 10 to about 21 base pairs, about 11 to about 20 base pairs, about 12 to about 19 base pairs, about 13 to about 18 base pairs, about 14 to about 17 base pairs, about or 15 to about 16 base pairs in length.

The barcode or derivative thereof may be detected. Detection of the barcode or derivative thereof may provide information related to the proximity of one analyte to another analyte. Detecting the barcode or derivative thereof may provide information related to the sequence of the barcode or derivative thereof. The information related to the sequence of the barcode or derivative thereof may be obtained across one or more cycles of detection. Each round of detection may provide information related to a portion of the barcode or derivative thereof. For example, a single round of detection may reveal sequence information for 2 nucleotides of a barcode or derivative thereof. Subsequent rounds of detection may reveal information for other nucleotides within the barcode or derivative thereof. The other nucleotides within the barcode or derivative thereof may be overlapping with the 2 nucleotides of the barcode or derivative thereof revealed in the single round or the other nucleotides within the barcode or derivative thereof may not be overlapping with the 2 nucleotides of the barcode or derivative thereof revealed in the single round. In some cases, a hybridization-based detection method may be used to detect the barcode or derivative thereof. For example, dye-labeled probes may be bound to the barcode or derivative thereof and reveal sequence information of the barcode or derivative thereof based on the sequence of the dye-labeled probes. In some cases, a sequencing-based detection method may be used to detect the barcode or derivative thereof.

Detecting the barcode or derivative thereof may comprise contacting the sample with materials to perform an in situ sequencing based reaction. A variety of in situ sequencing based reactions may be performed, including but not limited to sequencing by synthesis, Sequencing by Oligonucleotide Ligation and Detection (SOLiD), sequencing with error-reduction by dynamic annealing and ligation (SEDAL), or a combination thereof. Details of performing SEDAL sequencing can be found in PCT/US2019/025835, which is incorporated by reference in its entirety. In some cases, detecting the barcode or complement of the barcode may comprise in situ sequencing using a plurality of detection probes. In some embodiments, the complement of a barcode may comprise a reverse complement of the barcode.

After amplification, the sample may be contacted with a plurality of probes. The plurality of probes may comprise anchor probes and detection probes. An anchor probe may be used for detection of the barcode or derivative thereof sequence by hybridizing to a portion of the amplicon sequence that is adjacent to the binding site of a detection probe to enable a ligation event between one or more ends of one or more probes. The ligation between an anchor probe and a detection probe may increase the melting temperature associated with the ligated product as compared to the melting temperature of the detection probe alone. The increased melting temperature may provide enhanced stability of the duplex formed between the detection probe and the amplicon, and thereby enable more specific detection of the detection probe bound to the amplicon. The anchor probes may bind to the amplicon formed during amplification. An anchor probe of the anchor probes may bind to all or a portion of the barcode or derivative thereof. A detection probe may bind to all or a portion of the barcode or derivative thereof. The anchor probe of the anchor probes may bind to a sequence adjacent to the barcode or derivative thereof. The detection probe may bind to a sequence adjacent to the barcode or derivative thereof.

The detection probe may comprise a nucleic acid. The nucleic acid may comprise DNA, RNA, or a combination thereof. The nucleic acid of the detection probe may be single-stranded, double-stranded, or a combination thereof. The nucleic acid of the detection probe may comprise LNA. The detection probe may comprise a label. The label may be a detectable label, a linker, or a combination thereof. The detectable label may comprise a fluorescent molecule. The fluorescent molecule may comprise.

Detecting the barcode or reverse complement of the barcode may involve reading a signal associated with the barcode or reverse complement of the barcode. A variety of methods can use used to read a signal associated with the reverse complement of the barcode including hybridization-based detection, sequencing based detection, or a combination thereof. Reading a signal associated with the barcode or the reverse complement of the barcode may be performed in situ. One or more rounds of reading may be performed to collect one or more signals associated with the barcode or the reverse complement of the barcode. In some cases where one or more round of reading may be performed, the signal may be removed in between rounds.

In some cases, a detection probe may be added to the sample which binds to the barcode or reverse complement of the barcode. The detection probe may comprise a nucleic acid. The detection probe may comprise one or more labels. The label may comprise a one or more fluorescent molecules, one or more quantum dots, one or more proteins, one or more mass tags, one or more chromophores, or a combination thereof. In some cases, the one or more proteins may comprise an enzyme, e.g. a horseradish peroxidase. The enzyme may generate a signal indicative of the label. Examples of a fluorescent molecule include, but are not limited to AlexaFluor Texas Red, rhodamine B, rhodamine 6G, rhodamine 10, TMR-iodoacetamide, lissamine rhodamine B sulfonyl chloride, lissamine rhodamine B sulfonyl hydrazine, Texas Red sulfonyl chloride, Texas Red hydrazide, coumarin and coumarin derivatives such as AMCA, AMCA-NHS, AMCA-sulfo-NHS, AMCA-HPDP, DCIA, AMCE-hydrazide, BODIPY and derivatives such as BODIPY FL C3-SE, BODIPY 530/550 C3, BODIPY 530/550 C3-SE, BODIPY 530/550 C3 hydrazide, BODIPY 493/503 C3 hydrazide, BODIPY FL C3 hydrazide, BODIPY FL IA, BODIPY 530/551 IA, Br-BODIPY 493/503, Cascade Blue and derivatives such as Cascade Blue acetyl azide, Cascade Blue cadaverine, Cascade Blue ethylenediamine, Cascade Blue hydrazide, Lucifer Yellow, Lucifer Yellow CH, cyanine and derivatives such as indolium based cyanine dyes, benzo-indolium based cyanine dyes, pyridium based cyanine dyes, thiozolium based cyanine dyes, quinolinium based cyanine dyes, imidazolium based cyanine dyes, Cy 3, Cy5, lanthanide chelates and derivatives such as BCPDA, TBP, TMT, BHHCT, BCOT, Europium chelates, Terbium chelates, Alexa Fluor dyes, DyLight dyes, Atto dyes, LightCycler Red dyes, CAL Flour dyes, JOE and derivatives thereof, Oregon Green dyes, WellRED dyes, IRD dyes, phycoerythrin and phycobilin dyes, Malachite green, stilbene, DEG dyes, Cy3, Cy5, and Cy7, fluorescein and fluorescein derivatives such as carboxyfluorescein, tetrachlorofluorescein, hexachlorofluorescein, carboxynapthofluorescein, fluorescein isothiocyanate, NHS-fluorescein, iodoacetamidofluorescein, fluorescein maleimide, SAMSA-fluorescein, fluorescein thiosemicarbazide, carbohydrazinomethylthioacetyl-amino fluorescein, rhodamine and rhodamine derivatives such as TRITC, TMR, lissamine rhodamine, TEX 615, TYE™ 665, TYE 705, SUN, ATTO™ 425, ATTO™ 488, ATTO™ 532, ATTO™ 550, ATTO™ 565, ATTO™ Rho101, ATTO™ 590, ATTO™ 633, ATTO™ 647, ATTO™ 700, Alexa Fluor® 488 (NHS Ester), Alexa Fluor® 532 (NHS Ester), Alexa Fluor® 546 (NHS Ester), Alexa Fluor® 594 (NHS Ester), Alexa Fluor® 647 (NHS Ester), Alexa Fluor® 660 (NHS Ester), Alexa Fluor® 750 (NHS Ester), IRDye® 700, IRDye® 800, Rhodamine Red™, 5-TAMRA™, Texas Red®-X, Lightcycler® 640, Dy 750, or a combination thereof. The one or more labels may be connected to the detection probe at one or more ends, within the detection probe, or a combination hereof. In some cases, where the detection probe comprises a nucleic acid, the one or more labels may be connected to the nucleic acid at a 5′ end, at a 3′ end, internal within the nucleic acid, or a combination thereof.

The label of the detection probe may be connected to a nucleic acid with a linker. The linker may comprise a variety of chemical groups including one or more ethylene groups, one or more methylene groups, one or more poly-ethylene glycol groups, or a combination thereof. The detection probe may bind to the barcode or reverse complement of the barcode reversibly. The detection probe may bind to the barcode or reverse complement of the barcode irreversibly. The detection probe may bind to the barcode or reverse complement of the barcode through nucleic acid hybridization.

In some cases, one or more detection probes may be added to the sample. Each of the one or more detection probes may bind to the barcode or the reverse complement of the barcode. In some cases, each of the more one or more detection probes may bind to one or more barcodes or one or more reverse complements of a barcode in the sample. Each of the barcodes or reverse complements of the barcodes may relate to a different analyte or set of analytes. In some cases, each of the one or more detection probes may comprise a different label. In some cases, at least two of the detection probes may comprise a different label. In some cases, all of the one or more detection probes may comprise a different label. In some cases, the one or more detection probes may comprise the same label (e.g. the same fluorescent dye and/or the same linker).

In some cases, the one or more detection probes may be added to the sample and bind to one or more barcodes or reverse complements of one or more barcodes. A signal associated with the detection probe may be detected. Detection of the signal may be performed using an imaging system, e.g. an imaging system described herein. An imaging system may comprise a microscope, a camera, a stage, a sample holder, a computer, or a combination thereof. The imaging system may collect signal associated with the detection probe by illuminating the sample with light. The light used to illuminate the sample may comprise one or more wavelengths. The imaging system may collect light emitted by the sample at one or more wavelengths. The one or more wavelengths of illumination or collection may include, but is not limited to, light with the following wavelengths: at least about 260 nm, at least about 265 nm, at least about 270 nm, at least about 280 nm, at least about 285 nm, at least about 290 nm, at least about 295 nm, at least about 300 nm, at least about 305 nm, at least about 310 nm, at least about 315 nm, at least about 320 nm, at least about 325 nm, at least about 330 nm, at least about 335 nm, at least about 340 nm, at least about 345 nm, at least about 350 nm, at least about 355 nm, at least about 360 nm, at least about 365 nm, at least about 370 nm, at least about 375 nm, at least about 380 nm, at least about 385 nm, at least about 390 nm, at least about 395 nm, at least about 400 nm, at least about 405 nm, at least about 410 nm, at least about 415 nm, at least about 420 nm, at least about 425 nm, at least about 430 nm, at least about 435 nm, at least about 440 nm, at least about 445 nm, at least about 450 nm, at least about 455 nm, at least about 460 nm, at least about 465 nm, at least about 470 nm, at least about 475 nm, at least about 480 nm, at least about 485 nm, at least about 490 nm, at least about 495 nm, at least about 500 nm, at least about 505 nm, at least about 510 nm, at least about 515 nm, at least about 520 nm, at least about 525 nm, at least about 530 nm, at least about 535 nm, at least about 540 nm, at least about 545 nm, at least about 550 nm, at least about 555 nm, at least about 560 nm, at least about 565 nm, at least about 570 nm, at least about 575 nm, at least about 580 nm, at least about 585 nm, at least about 590 nm, at least about 595 nm, at least about 600 nm, at least about 605 nm, at least about 610 nm, at least about 615 nm, at least about 620 nm, at least about 625 nm, at least about 630 nm, at least about 635 nm, at least about 640 nm, at least about 645 nm, at least about 650 nm, at least about 655 nm, at least about 660 nm, at least about 665 nm, at least about 670 nm, at least about 675 nm, at least about 680 nm, at least about 685 nm, at least about 690 nm, at least about 695 nm, at least about 700 nm, at least about 705 nm, at least about 710 nm, at least about 715 nm, at least about 720 nm, at least about 725 nm, at least about 730 nm, at least about 735 nm, at least about 740 nm, at least about 745 nm, at least about 750 nm, at least about 755 nm, at least about 760 nm, at least about 765 nm, at least about 770 nm, at least about 775 nm, at least about 780 nm, at least about 785 nm, at least about 790 nm, at least about 795 nm, at least about 800 nm, or more nm. The one or more wavelengths of illumination or collection may comprise light with the following wavelengths: at most about 260 nm, at most about 265 nm, at most about 270 nm, at most about 280 nm, at most about 285 nm, at most about 290 nm, at most about 295 nm, at most about 300 nm, at most about 305 nm, at most about 310 nm, at most about 315 nm, at most about 320 nm, at most about 325 nm, at most about 330 nm, at most about 335 nm, at most about 340 nm, at most about 345 nm, at most about 350 nm, at most about 355 nm, at most about 360 nm, at most about 365 nm, at most about 370 nm, at most about 375 nm, at most about 380 nm, at most about 385 nm, at most about 390 nm, at most about 395 nm, at most about 400 nm, at most about 405 nm, at most about 410 nm, at most about 415 nm, at most about 420 nm, at most about 425 nm, at most about 430 nm, at most about 435 nm, at most about 440 nm, at most about 445 nm, at most about 450 nm, at most about 455 nm, at most about 460 nm, at most about 465 nm, at most about 470 nm, at most about 475 nm, at most about 480 nm, at most about 485 nm, at most about 490 nm, at most about 495 nm, at most about 500 nm, at most about 505 nm, at most about 510 nm, at most about 515 nm, at most about 520 nm, at most about 525 nm, at most about 530 nm, at most about 535 nm, at most about 540 nm, at most about 545 nm, at most about 550 nm, at most about 555 nm, at most about 560 nm, at most about 565 nm, at most about 570 nm, at most about 575 nm, at most about 580 nm, at most about 585 nm, at most about 590 nm, at most about 595 nm, at most about 600 nm, at most about 605 nm, at most about 610 nm, at most about 615 nm, at most about 620 nm, at most about 625 nm, at most about 630 nm, at most about 635 nm, at most about 640 nm, at most about 645 nm, at most about 650 nm, at most about 655 nm, at most about 660 nm, at most about 665 nm, at most about 670 nm, at most about 675 nm, at most about 680 nm, at most about 685 nm, at most about 690 nm, at most about 695 nm, at most about 700 nm, at most about 705 nm, at most about 710 nm, at most about 715 nm, at most about 720 nm, at most about 725 nm, at most about 730 nm, at most about 735 nm, at most about 740 nm, at most about 745 nm, at most about 750 nm, at most about 755 nm, at most about 760 nm, at most about 765 nm, at most about 770 nm, at most about 775 nm, at most about 780 nm, at most about 785 nm, at most about 790 nm, at most about 795 nm, at most about 800 nm, or less nm. The one or more wavelengths of illumination or collection may comprise light with the following wavelengths about 260 to about 800 nm, about 265 to about 795 nm, about 270 to about 790 nm, about 280 to about 785 nm, about 285 to about 780 nm, about 290 to about 775 nm, about 295 to about 770 nm, about 300 to about 765 nm, about 305 to about 760 nm, about 310 to about 755 nm, about 315 to about 750 nm, about 320 to about 745 nm, about 325 to about 740 nm, about 330 to about 735 nm, about 335 to about 730 nm, about 340 to about 725 nm, about 345 to about 720 nm, about 350 to about 715 nm, about 355 to about 710 nm, about 360 to about 705 nm, about 365 to about 700 nm, about 370 to about 695 nm, about 375 to about 690 nm, about 380 to about 685 nm, about 385 to about 680 nm, about 390 to about 675 nm, about 395 to about 670 nm, about 400 to about 665 nm, about 405 to about 660 nm, about 410 to about 655 nm, about 415 to about 650 nm, about 420 to about 645 nm, about 425 to about 640 nm, about 430 to about 635 nm, about 435 to about 630 nm, about 440 to about 625 nm, about 445 to about 620 nm, about 450 to about 615 nm, about 455 to about 610 nm, about 460 to about 605 nm, about 465 to about 600 nm, about 470 to about 595 nm, about 475 to about 590 nm, about 480 to about 585 nm, about 485 to about 580 nm, about 490 to about 575 nm, about 495 to about 570 nm, about 500 to about 565 nm, about 505 to about 560 nm, about 510 to about 555 nm, about 515 to about 550 nm, about 520 to about 545 nm, about 525 to about 540 nm, or about 530 to about 535 nm.

The one or more detection probes may bind to the sample and detect a sequence associated with the barcode or the reverse complement of the barcode. In some cases, the sequence associated with the barcode, or the reverse complement of the barcode, may comprise the entire sequence of a barcode ore reverse complement of the barcode. In some cases, the sequence associated with the barcode, or the reverse complement of the barcode, may comprise a portion of the sequence of a barcode ore reverse complement of the barcode. In some cases, the detection probe detects at least about 1 nucleotide, at least about 2 nucleotides, at least about 3 nucleotides, at least about 4 nucleotides, at least about 5 nucleotides, at least about 6 nucleotides, at least about 7 nucleotides, at least about 8 nucleotides, at least about 9 nucleotides, at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, at least about 20 nucleotides, at least about 21 nucleotides, at least about 22 nucleotides, at least about 23 nucleotides, at least about 24 nucleotides, at least about 25 nucleotides, at least about 26 nucleotides, at least about 27 nucleotides, at least about 28 nucleotides, at least about 29 nucleotides, at least about 30 nucleotides, at least about 31 nucleotides, at least about 32 nucleotides, at least about 33 nucleotides, at least about 34 nucleotides, at least about 35 nucleotides, at least about 36 nucleotides, at least about 37 nucleotides, at least about 38 nucleotides, at least about 39 nucleotides, at least about 40 nucleotides, or more nucleotides associated with the barcode or the reverse complement of the barcode. In some cases, the detection probe detects at most about 1 nucleotide, at most about 2 nucleotides, at most about 3 nucleotides, at most about 4 nucleotides, at most about 5 nucleotides, at most about 6 nucleotides, at most about 7 nucleotides, at most about 8 nucleotides, at most about 9 nucleotides, at most about 10 nucleotides, at most about 11 nucleotides, at most about 12 nucleotides, at most about 13 nucleotides, at most about 14 nucleotides, at most about 15 nucleotides, at most about 16 nucleotides, at most about 17 nucleotides, at most about 18 nucleotides, at most about 19 nucleotides, at most about 20 nucleotides, at most about 21 nucleotides, at most about 22 nucleotides, at most about 23 nucleotides, at most about 24 nucleotides, at most about 25 nucleotides, at most about 26 nucleotides, at most about 27 nucleotides, at most about 28 nucleotides, at most about 29 nucleotides, at most about 30 nucleotides, at most about 31 nucleotides, at most about 32 nucleotides, at most about 33 nucleotides, at most about 34 nucleotides, at most about 35 nucleotides, at most about 36 nucleotides, at most about 37 nucleotides, at most about 38 nucleotides, at most about 39 nucleotides, at most about 40 nucleotides, or fewer nucleotides associated with the barcode or the reverse complement of the barcode. In some cases, the detection probe detects about 1 to about 40 nucleotides, about 2 to about 39 nucleotides, about 3 to about 38 nucleotides, about 4 to about 37 nucleotides, about 5 to about 36 nucleotides, about 6 to about 35 nucleotides, about 7 to about 34 nucleotides, about 8 to about 33 nucleotides, about 9 to about 32 nucleotides, about 10 to about 31 nucleotides, about 11 to about 30 nucleotides, about 12 to about 29 nucleotides, about 13 to about 28 nucleotides, about 14 to about 27 nucleotides, about 15 to about 26 nucleotides, about 16 to about 25 nucleotides, about 17 to about 24 nucleotides, about 18 to about 23 nucleotides, about 19 to about 22 nucleotides, or about 20 to about 21 nucleotides associated with the barcode or the reverse complement of the barcode.

Additional probes may be added to the sample during the detection steps. In some cases, one or more anchor probes may be added to the sample. The one or more anchor probes may bind to all or a portion of the barcode or the reverse complement of the barcode. The one or more anchor probes may bind to a region adjacent to the barcode or reverse complement of the barcode. The region adjacent to the barcode or reverse complement of the barcode may be part of a nucleic acid. The nucleic acid may be an amplicon or amplification product. In some cases, the one or more anchor probe may bind to both the barcode and a sequence adjacent to the barcode. In some cases, the one or more anchor probes may bind to both the reverse complement of the barcode and a sequence adjacent to the reverse complement. The one or more anchor probes may comprise a nucleic acid. The nucleic acid may comprise RNA, DNA, or a combination thereof. The nucleic acid may comprise one or more modifications. The one or more modifications may comprise a phosphorylation modification, an LNA, or a combination thereof. The phosphorylation modification may be a 5′ phosphorylation modification. In some cases, a detection probe and an anchor probe are hybridized to an amplicon generated from rolling circle amplification. The detection probe and the anchor probe may be ligated after hybridization to the amplicon.

The one or more anchor probes may bind to the barcode or reverse complement of the barcode, or sequence adjacent to the barcode or reverse complement of the barcode such that the one or more anchor probes is adjacent to one or more detection probes. The one or more anchor probes may be directly adjacent to the one or more detection probes, such that there are no intervening nucleotides. In some cases, the one or more anchor probes may be adjacent to the one or more detection probes such that there is one or more intervening nucleotides. A gap-filling reaction may be performed to fill in the intervening nucleotides between one or more anchor probes and one or more detection probes. The one or more anchor probes may be ligated to the one or more detection probes. The ligation between an anchor probe and a detection probe may be performed using enzymatic ligation, chemical ligation, or a combination thereof. The enzymatic ligation may be performed by a ligase. The ligase may comprise one or more of the following: T4 DNA ligase, SplintR ligase, T3 DNA ligase, T7 DNA ligase, E. coli DNA ligase, Taq ligase, RtcB ligase, or a combination thereof.

Detecting a signal associated with one or more detection probes may be performed after ligation of one or more detection probes with one or more anchor probes. In some cases, the one or more detection probes may transiently bind to the sample. In some cases, the one or more detection probes may bind to the sample for a duration of time without appreciable dissociation. The one or more detection probes ligated to one or more anchor probes may bind to the sample more strongly than the one or more detection probes alone.

In some cases, after signal detection, one or more labels associated with the sample may be removed. The label may be removed from the sample with chemical or enzymatic means. In some cases, the label may be cleaved from the detection probe, using an enzyme, a chemical cleavage reagent, or a combination thereof. The enzyme may comprise a restriction enzyme, a polymerase, a ligase, a transposon, on a combination thereof. The chemical cleavage reagent may comprise a reducing reagent, a reactive oxygen species, or a combination thereof. The label may be removed from the sample by removing, digesting or disrupting the one or more detection probes from the sample. In some cases, the one or more detection probes may be ligated to one or more anchor probes. The one or more detection probes may be removed from the sample using temperature and incubating the sample at an elevated temperature for a duration of time. The one or more detection probes may be removed from the sample by incubating the sample with a chemical that disrupts nucleic acid hybridization, e.g. one or more chaotropic agent. In some cases, the one or more chaotropic agents may comprise glycine, arginine, histidine hydrochloride, sodium hydroxide, formamide, dimethyl sulfoxide (DMSO), guanidinium chloride, or a combination thereof. The one or more detection probes may be removed from the sample by incubating the sample with one or more chaotropic reagents and incubating the sample at an elevated temperature. The one or more detection probes may be removed from the sample using an enzyme, including but not limited to a DNAse, an RNAse, a restriction enzyme, or a combination thereof.

In some cases, after signal detection, one or more labels associate with the sample may be altered such that the label is no longer capable of emitting a signal. The label may be denatured, e.g. in cases where the label may comprise a polypeptide. Denaturation may be performed using heat, salt, a solvent (e.g. methanol, ethanol, or combination thereof), an acid, a base, or a combination thereof. The label may be photobleached, e.g. in cases where the label may comprise a fluorescent moiety. Photobleaching may be performed by exposing the sample to light, including ambient light, to a reactive chemical species, a reducing reagent, a base, an acid, or a combination thereof.

One or more rounds of detection may be performed. Each round of detection may comprise contacting the sample with one or more detection probes, one or more anchor probes, or a combination thereof. For example, a sample may comprise an amplicon comprising a reverse complement of a barcode sequence comprising 8 nucleotides. A round of detection may comprise adding a plurality of anchor probes and a plurality of detection probes to the sample to enable binding of anchor probes and detection probes to the amplicon, where the sequence of the detection probe provides information related to the first two nucleotides of the 8 nucleotide reverse complement of the barcode sequence. After binding, the anchor probe and detection probe may be ligated, and the sample may be washed to remove all unbound detection probes. The sample may be imaged to detect the signal associated with the detection probe. After imaging, the detection probe ligated to the anchor probe may be removed by incubation of the sample with a chaotropic reagent. The combination of binding of the detection probe, ligating the detection probe, imaging the sample, and removing the detection probe comprise a round of detection in this case. In some cases, the one or more detection probes may be the same across one or more rounds. For example, detection probes with the same sequence may be added in a first and a second cycle. In some cases, the one or more detection probes may be different across one or more rounds. For example, detection probes with difference sequences may be added in a first round as compared to a second round. In some cases, the one or more anchor probes may be the same across one or more rounds. For example, anchor probes with the same sequence may be added in a first and second cycle. In some cases, the one or more anchor probes may be different across one or more rounds. For example, anchor probes with difference sequences may be added in a first round as compared to a second round. Each round of detection may comprise adding probes to the sample, imaging the sample using an imaging system to generate one or more images, optionally, and removing the label from the sample.

One or more images generated by imaging the sample during a detection round as described may be analyzed. The images may comprise information related to the sequence of a barcode or reverse complement of a barcode. In some cases, one or images from one round of detection may comprise information related to a portion of the sequence of one or more barcodes or one or more reverse complements of a barcode. In some cases, one or more images from another round of detection may comprise information related to another portion of the sequence or one or more barcodes or one or more reverse complements of a barcode. Analyzing images generated during one or more rounds of detection may result in identification of one or more barcodes or one or more reverse complement of one or more barcodes. Identification of one or more barcodes or one or more reverse complements of one or more barcodes may enable identification of one or more targets, as described herein. A spatial map, image, display, summary, table, or combination thereof may be generated based on the analysis described herein. Information related to both the identity of a target and the spatial location, may be determined based on the analysis described herein. The spatial location may comprise information related to the location of a target in an x-direction, y-direction, z-direction, or a combination thereof.

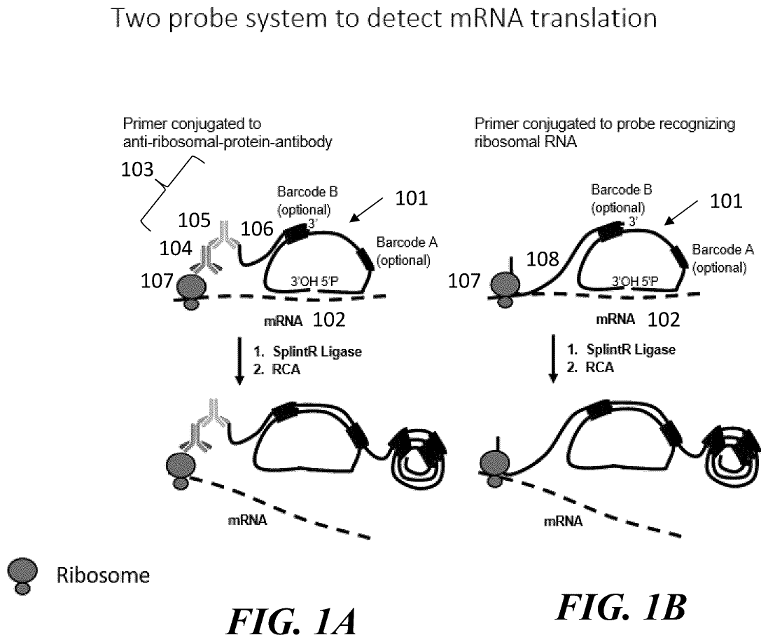

In some cases, the first probe may recognize a messenger RNA and the second probe may recognize a ribosomal protein. A proximity of the messenger RNA and the ribosomal protein may be determined. The proximity of the messenger RNA and the ribosomal protein may provide information related to the translation of a protein. For example, a barcode or derivative thereof may be detected as a result of the messenger RNA and the ribosomal protein being in close proximity to each other. The close proximity of the messenger RNA and the ribosomal protein may cause a ligation event of one end of nucleic probe to another end of the nucleic acid probe, which may lead to the generation of multiple copies of the barcode or derivative thereof. The detection of the barcode or derivative thereof my indicate that the messenger RNA is part of a complex with the ribosomal protein, which may occur during translation of a protein encoded by the messenger RNA. An example of this type of detection is shown in FIG. 1A and FIG. 1B, which depict two probe systems to detect messenger RNA (mRNA) translation. The first detection scheme, shown in FIG. 1A, comprises a first probe (101) that recognizes a messenger RNA (102) and a second probe (103), which comprises an antibody (104) that recognizes a ribosome (107). In this case, the second probe comprises a primary antibody (104), and a secondary antibody (105) conjugated to a nucleic acid (106) (e.g. a primer). The primary antibody may be an anti-ribosomal protein antibody. The nucleic acid conjugated to the secondary antibody may comprise a fourth binding site, which binds to a binding site (e.g. a third binding site of the first probe) on the first probe. The first probe may comprise a 5′-phosphorylated base and a 3′-free deoxyribose that may be complimentary to an adjacent position on the target oligo (mRNA) and may anneal to the nucleic acid conjugated to the antibody. The first probe may comprise two optional barcodes, here depicted as Barcode A and Barcode B. The second probe may comprise Barcode B. In the cases that there is a gap between the two ends, the gap may be filled by DNA bases using Reverse Transcriptase-mediated polymerization. The nick between the 5′ and 3′ end of the first probe may be ligated in the presence of the mRNA using a ligase (e.g. SplintR ligase), which may produce a circular oligonucleotide. The circular oligonucleotide may serve as the template for a rolling circle amplification (RCA) reaction using a DNA polymerase (e.g. Phi29 polymerase). In this case, the nucleic acid conjugated to the secondary antibody may serve as the primer to initiate the RCA reaction. The antibody may recognize a ribosomal component directly (e.g. when a nucleic acid is conjugated to the primary antibody that recognizes ribosomal proteins) or indirectly (e.g. when a nucleic acid is conjugated to the secondary antibody that recognizes ribosomal proteins). The scheme shown in FIG. 1B involves use of a nucleic acid probe (108) that recognizes a Ribosomal RNA. In each case depicted in FIG. 1A and FIG. 1B, The RCA product may be embedded in a hydrogel, which may preserve the spatial arrangement of nucleic acids within the sample. An optional Barcode A in the first probe can be specific to each target nucleic acid. An optional barcode B in the first probe and second probe may be specific to the protein that antibody binds to. Barcode A or B or the complement thereof may be identified through ligation-based or hybridization-based or sequencing-based in situ sequencing with fluorophore-labeled probes that may be visible under a microscope using a laser.

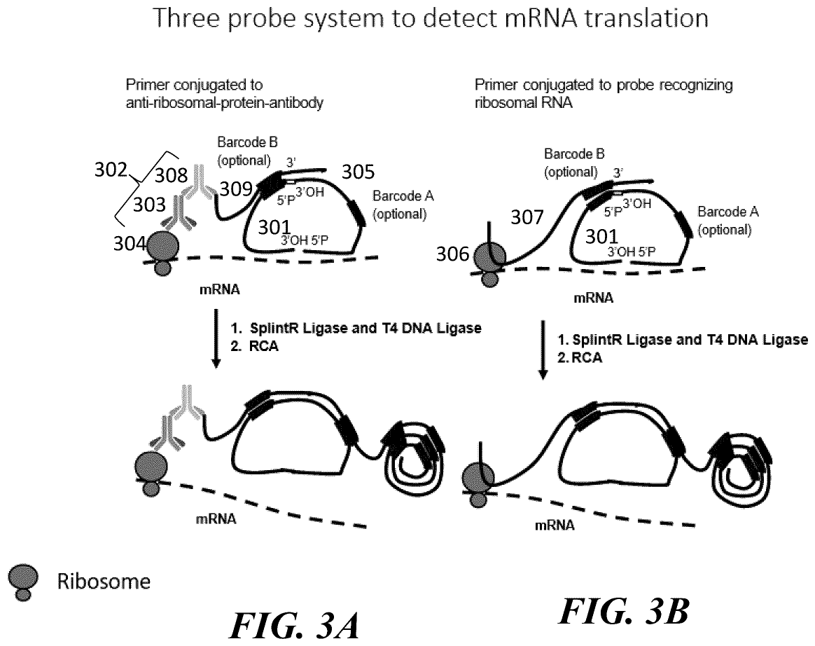

Another set of examples of detecting mRNA translation is shown in FIGS. 3A and 3B. In this case, three probes are used to detect mRNA translation by detecting the presence of a ribosome in proximity to an mRNA. The detection of a ribosome and an mRNA in FIG. 3A is similar to that of FIG. 1A, with one difference being the inclusion of an additional probe (e.g. a third probe) (301). The additional probe in this case may bind to the mRNA and a nucleic acid conjugated to a secondary probe (302), which may be bound to a primary antibody (303), which may be bound to a ribosome (304). The primary antibody may be bound to a secondary antibody (308) conjugated to an oligonucleotide (309) that binds to the first probe and the third probe. This additional probe may ligate to the first probe (305) at two locations. The additional probe may comprise a 5′ phosphorylation modification and a 3′ hydroxyl group. The 5′ phosphorylation modification my ligate to a 3′ hydroxyl group of the first probe at a binding site to the nucleic acid conjugated to the secondary antibody. The additional probe (e.g. third probe) may ligate to the first probe (305) at a second location, for example a binding site on the mRNA. In some cases, there may be a gap between the ends of the first probe the third probe and a gap-filling reaction may be performed. The first probe may comprise an optional Barcode A. The additional probe (e.g. third probe) may comprise an optional Barcode B. The nucleic acid conjugated to a secondary antibody may comprise an optional Barcode B. In some cases, there may be a gap between two ends of the probes. The gap between two ends of the probes may be filled by DNA bases through DNA polymerase-mediated polymerization. The nick between the 5′ end of the first probe and 3′ end of the third probe may bind the target (e.g. the mRNA), or detection-moiety-conjugated Nucleotide primer (e.g. the second probe) and may be ligated. The ligation between the third probe and the first probe may be performed by SplintR ligase, T4 DNA ligase, or a combination thereof. The ligation may produce a circular oligonucleotide that may serve as the template for an RCA reaction by DNA polymerase. The detection-moiety-conjugated Nucleotide primer (e.g. second probe) may serve as the primer to initiate the RCA reaction. When the detection moiety is an antibody, the antibody may recognize a ribosomal component directly (when oligo is conjugated to the primary antibody that recognizes ribosomal proteins) or indirectly (when oligo is conjugated to the secondary antibody that recognizes ribosomal proteins). The ribosome component may be a ribosomal protein. When the detection moiety is a nucleic acid probe, e.g. a ribosomal RNA (306), the nucleic acid probe (e.g. second probe) (307) may bind to the ribosomal RNA, as shown in FIG. 3B. The RCA product may be embedded in a hydrogel. An optional Barcode A in the first probe may (308) be specific to each target nucleic acid. An optional barcode B in the first probe and second probe may be specific to the protein that the antibody binds to. Barcode A may be specifically identified through ligation-based or hybridization-based in situ sequencing with fluorophore-labeled oligo that is visible under a microscope using laser.

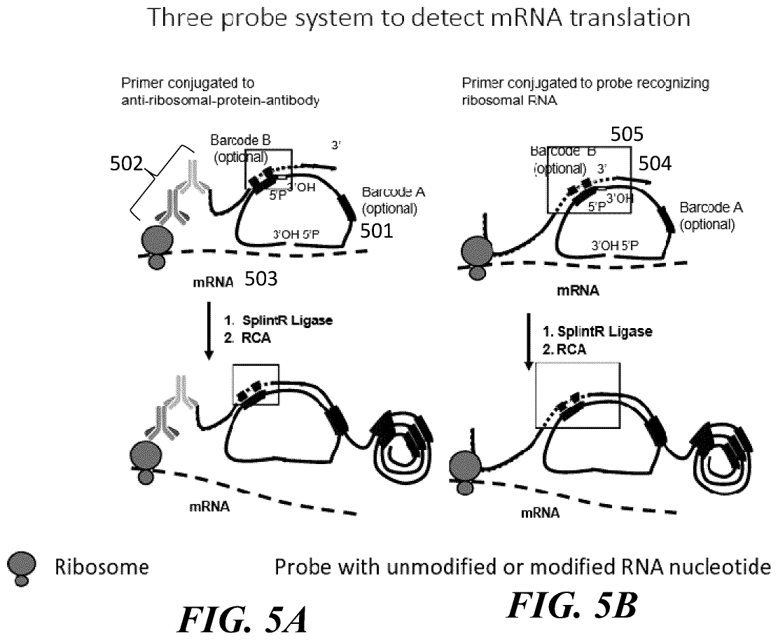

Another set of examples of detecting mRNA translation is shown in FIG. 5A and FIG. 5B. In this case, the primer probe containing an unmodified or modified RNA chimeric sequence may serve as a template for SplintR-mediated DNA ligation between the first probe (501) and second probe (502). Both the first probe and second probe may comprise a 5′-phosphorylated base and a 3′-free deoxyribose. The 5′ of the first probe and the 3′ of the second probe may be complimentary to an adjacent position on the target mRNA (503) (e.g. mRNA, “nucleotide of interest”) and may anneal to the target mRNA (503). The 5′ of a second probe (501) 2 and a 3′ of the first probe (501) may be complimentary to an adjacent position on the detection-moiety-conjugated Nucleotide primer (e.g. second probe). In the cases that there is a gap between the two ends, the gap may be filled by DNA bases through DNA polymerase-mediated polymerization. The nick between the 5′ and 3′ end of the first probe and second probe may anneal on the target oligo or antibody-conjugated Nucleotide primer and may be ligated by SplintR ligase and/or T4 DNA ligase. The ligation may produce a circular oligonucleotide. The circular oligonucleotide may serve as a template for an RCA reaction by DNA polymerase. The detection-moiety-conjugated Nucleotide primer (e.g. the second probe) may serve as the primer to initiate the RCA reaction. When the detection moiety is an antibody, the antibody may recognize a ribosomal component directly (when oligo is conjugated to the primary antibody that recognizes ribosomal proteins) or indirectly (when oligo is conjugated to the secondary antibody that recognizes ribosomal proteins). When the detection moiety is a nucleic acid probe, the nucleic acid probe may recognize Ribosomal RNA. The RCA product can be embedded in a hydrogel. An optional barcode A in the first probe may be specific to each target nucleic acid. An optional barcode B of the first probe and antibody-conjugated Nucleotide primer may be specific to the protein that antibody binds to. Barcode A may be specifically identified through ligation-based or hybridization-based in situ sequencing with fluorophore-labeled oligo that is visible under a microscope using laser. FIG. 5B depicts a similar workflow but includes an additional gap-filling reaction (505) to connect the second probe with an additional probe (504) that binds the first probe.

In some cases, the first probe may recognize a messenger RNA and the second probe may recognize a ribosomal RNA. A proximity of the messenger RNA and the ribosomal RNA may be determined. For example, a barcode or derivative thereof that may be amplified as a result of a ligation reaction between two ends of the first probe based on binding of the first probe to the messenger RNA, the second probe to the ribosomal RNA, and the first probe to the second probe may be detected (e.g. using an imaging system). The proximity of the messenger RNA and the ribosomal RNA may provide information related to the translation of a protein. For example, detecting the presence of the barcode or derivative thereof may indicate that the messenger RNA is complexed to the ribosomal RNA, which may be an indicated that the protein encoded by the messenger RNA is being translated.

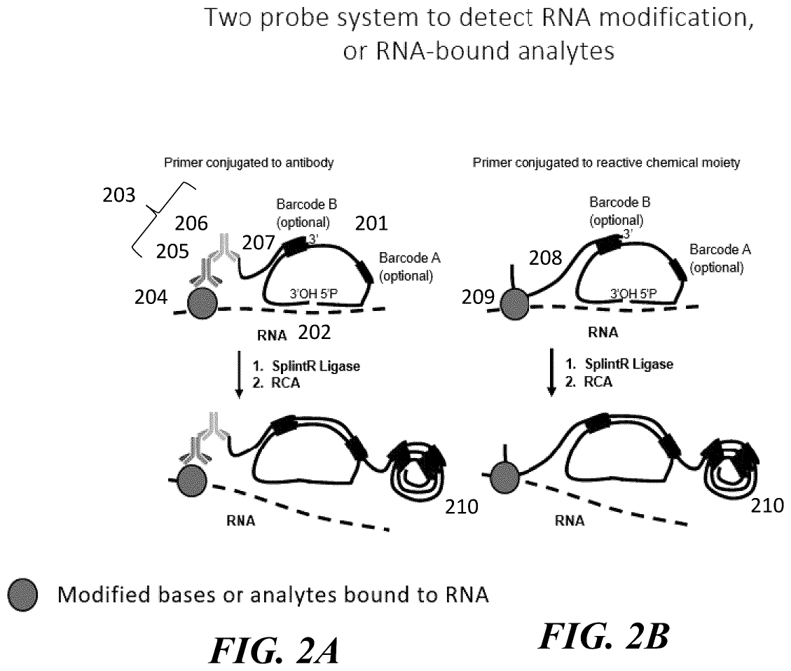

In some cases, the first probe may recognize a messenger RNA and the second probe may recognize, couple to, and/or bind a messenger RNA modification. The RNA modification may be incorporated by metabolic labeling, for example. A proximity of the messenger RNA and the messenger RNA modification may be determined. The proximity of the messenger RNA and the messenger RNA modification may provide information related to the translation of a protein, transcription of the mRNA, or a combination thereof. The second probe may comprise a reactive chemical moiety. The second probe may comprise an antibody or antibody fragment. An example of this type of detection is depicted in FIG. 2A and FIG. 2B, which shows a two-probe system to detect RNA modification or RNA-bound analytes. FIG. 2A shows the first probe (201) binding an RNA (202) (e.g. a messenger RNA), and the second probe (203) binding one or more modified bases (e.g. an RNA modification) or an analyte bound to the RNA (204). In this case, the second probe comprises an antibody (205) that recognizes the one or more modified bases (e.g. an RNA modification) or an analyte bound to the RNA (204) and a secondary antibody (206) conjugated to a nucleic acid (207), which may comprise a fourth binding site. The fourth binding site may be configured to bind to the first probe at a third binding site. The first probe (201) may bind to the RNA at a first and/or a second binding site. The first probe may comprise a 5′-phosphorylated base and/or a 3′-free deoxyribose that may be complimentary to an adjacent position on the first probe and can anneal to the first probe. The first probe may comprise one or more optional barcodes (e.g. two barcodes). In this case, two barcodes are depicted as Barcode A and Barcode B. The second probe may comprise Barcode B. In some cases, there may be a gap between two ends of the first probe. the gap may be filled by DNA bases through Reverse Transcriptase-mediated polymerization. The nick between the 5′ and 3′ end of oligo 1 may be ligated in the presence of target oligo by SplintR ligase and/or DNA ligase. The ligation may produce a circular oligonucleotide that may serve as a template for an RCA reaction by DNA polymerase. In this case, the nucleic acid conjugated to the secondary antibody may serve as the primer to initiate the RCA reaction. In the detection scheme shown in FIG. 2B, the second probe (208) comprises a nucleic acid and may bind one or more modifications or analytes bound to the RNA (209). The second probe may serve as the primer to initiate the RCA reaction. In both cases shown in FIG. 2A and FIG. 2B, one or more amplicons may be generated (210). The one or more amplicons may comprise copies of Barcode A, and/or Barcode B, or a complement thereof. The RCA product (e.g. one or more amplicons) may be embedded in a hydrogel, which may preserve the spatial arrangement of nucleic acids within the sample. Barcode A or B or the complement thereof may be identified through ligation-based or hybridization-based or sequencing-based in situ sequencing with fluorophore-labeled probes that may be visible under a microscope using a laser.

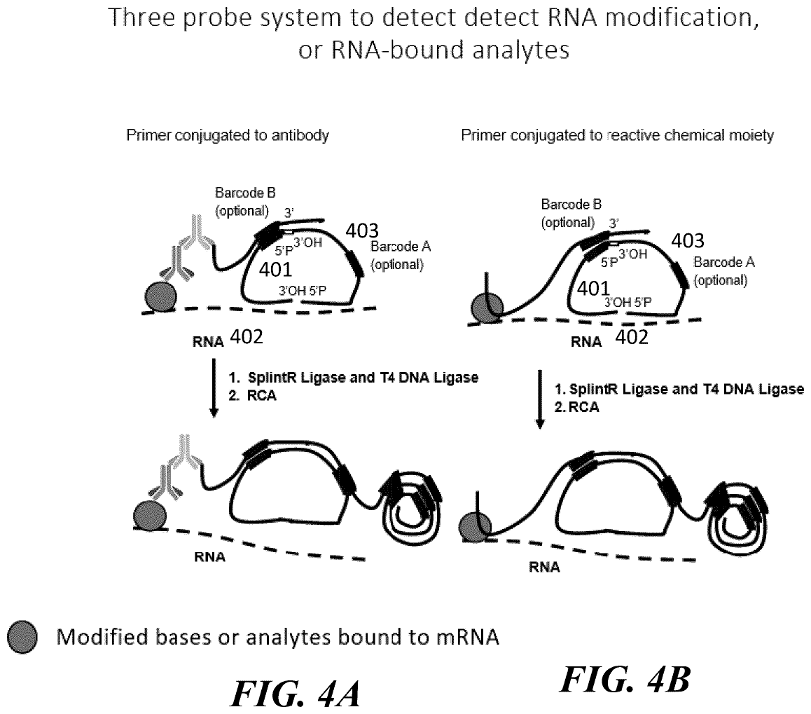

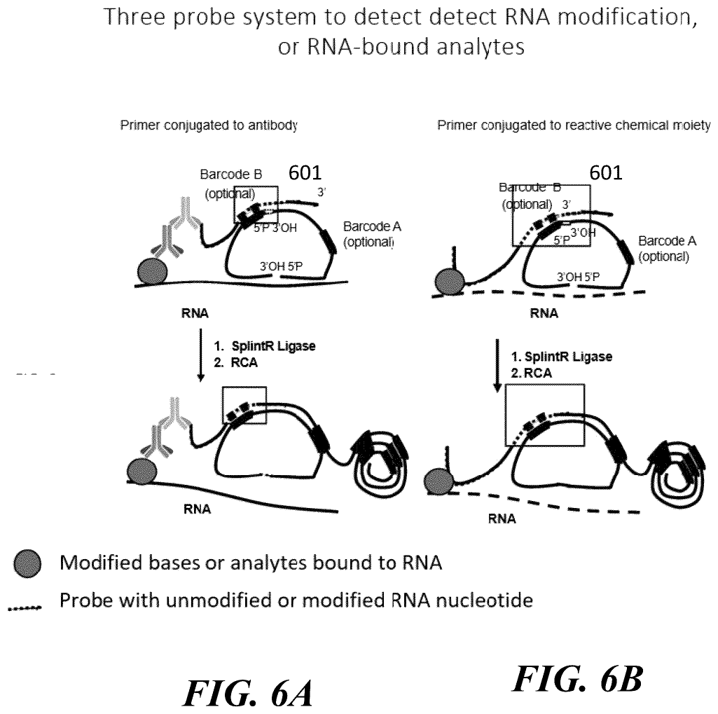

Another example of detecting the proximity of an RNA and an RNA modification is shown in FIG. 4. The detection scheme in FIG. 4A and FIG. 4B is similar to that shown in FIG. 2A and FIG. 2B, with the addition of a third probe (401) used to detect a target analyte (e.g. an RNA). In this case, the third probe may bind the analyte of interest, which may be an RNA (402). The RNA bound by the third probe and first probe (403) may comprise a modified base or analyte bound to mRNA, as described in FIG. 2A and/or FIG. 2B. The additional steps of the workflow of FIGS. 4A and 4B are similar to those shown in FIGS. 2A and 2B. Another example of detecting the proximity of an RNA and an RNA modification is shown in FIGS. 6A and 6B. The primer probe containing an unmodified or modified RNA chimeric sequence that can serve as a template for SplintR-mediated DNA ligation of the second probe (601) to another probe. The detection-moiety conjugated to the nucleotide primer may be an antibody, probe or chemical functional groups that recognize or react with the target analyte that is either part of the RNA or bind to the RNA. The workflow in FIG. 6 may be similar to that shown in FIG. 2 and/or FIG. 4, with the addition of a gap-filling reaction to generate a primer to initiate an RCA reaction.

In some cases, the first probe may recognize a DNA and the second probe may recognize a DNA modification. A proximity of the DNA and the DNA modification may be determined. The proximity of the DNA and the DNA modification may provide information related to the transcriptional state of RNA encoded by the DNA, translational state of a protein encoded by the DNA, or a combination thereof. The second probe may comprise a reactive chemical moiety. The second probe may comprise an antibody or antibody fragment.

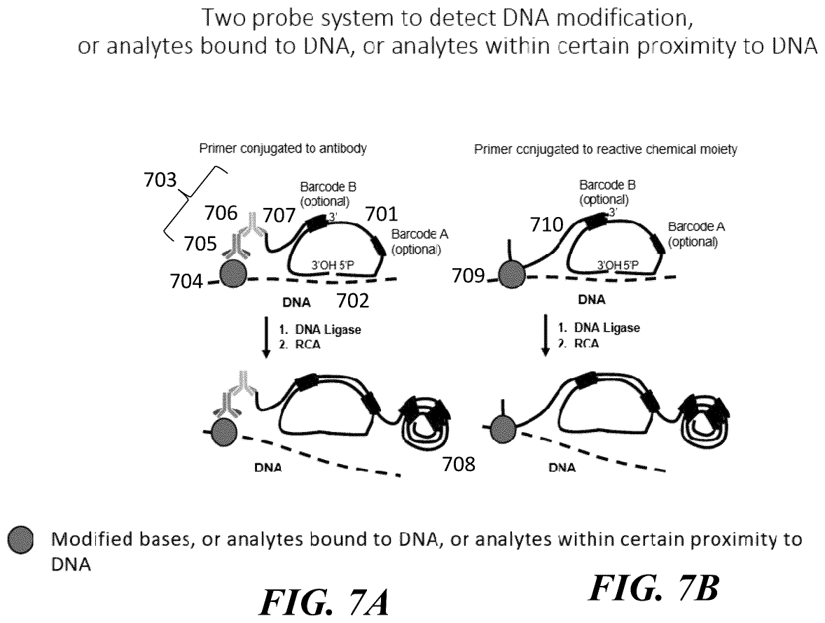

An example of detecting the proximity of a DNA modification and/or analytes bound to DNA to a DNA is shown in FIG. 7A and FIG. 7B. As shown in FIG. 7A, a first probe (701) may bind to a DNA (702). A second probe (703) may bind to a DNA modification (704). The DNA modification may be on the same DNA molecule recognized by the first probe (701). In some cases, the second probe may comprise an antibody (705) that binds to the DNA modification. In some cases, the second probe may comprise a secondary antibody (706) that binds the antibody that binds to the DNA modification. The secondary antibody may comprise a primer conjugated to it (707). The primer of the secondary antibody may serve as a primer to initiate an RCA reaction. The first probe may be ligated after binding the DNA template. (e.g. the DNA analyte of interest). The ligation may result in the generation of a circular oligonucleotide. The circular oligonucleotide may serve as the template for an RCA reaction. The RCA reaction may generate one or more amplicons (708.

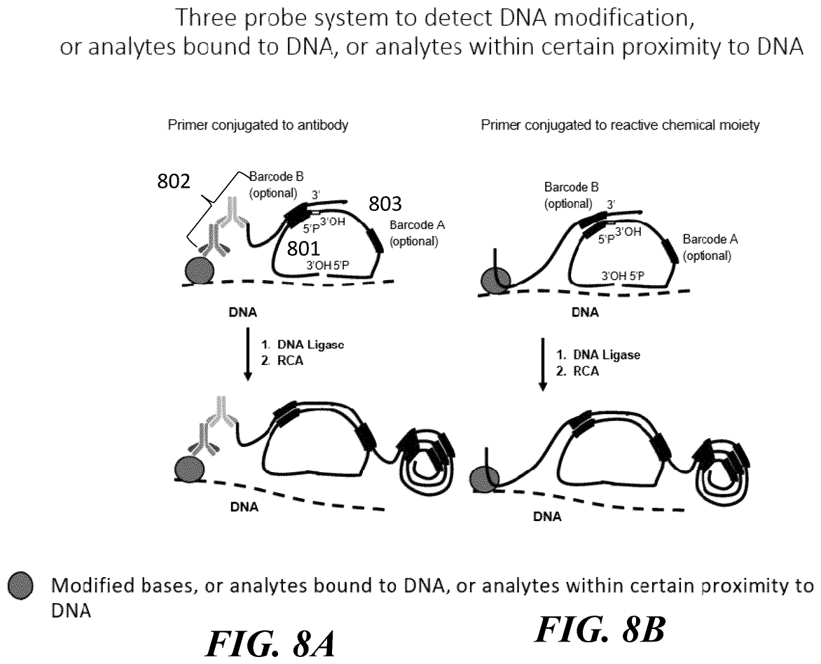

The one or more amplicons may be detected using one or more detection probes. The workflow shown in FIG. 7B is similar to that shown in FIG. 7A, but in this case the second probe (710) comprises a nucleic acid that may hybridize to a DNA modification containing sequence (709) of the same sequence as the DNA sequence bound by the first probe. Another example of detecting the proximity of a DNA modification and/or analytes bound to DNA to a DNA is shown in FIG. 8A and FIG. 8B. The detection scheme shown in FIG. 8A shows a three-probe system to detect DNA modifications and/or analytes bound to DNA in proximity to DNA. The detection scheme in this case is similar to that shown in FIG. 7A with the addition of a third probe (801). The third probe may bind to the DNA and/or second probe (802). A ligation reaction may be performed between the first probe (803) and the third probe (801) at one or more locations to generate a circular oligonucleotide. The circular oligonucleotide may serve as a template for an RCA reaction to generate one or more amplicons.

Described herein also is a detection-moiety-conjugated primer probe system, comprising one or more oligonucleotide probes (Nucleotide primer probe(s)). At least one 3′OH end and at least one 5′P end may be complementary to an mRNA upon annealing to a complementary nucleotide (mRNA) sequence. Annealing the detection moiety conjugated primer probe to the mRNA sequence may place the 3′OH and 5′P ends within 0-100 nucleobases of each other. Extension of nucleobases can be performed by reverse transcriptase to lead to an adjacent 3′OH and 5′P end annealed to mRNA. The 3′OH and the 5′P ends may ligate together to generate a circular oligonucleotide. The circular oligonucleotide may comprise at least one unique barcode region. The circular oligonucleotide may be amplified.

In some embodiments, the amplification may be accomplished by rolling circle amplification. In some embodiments, the first probe may comprise a 5′ phosphorylation modification. In some embodiments the first probe may comprise a 3′ hydroxyl group. In some cases, the second probe may comprise a 5′ phosphorylation modification. In some embodiments the second probe may comprise a 3′ hydroxyl group. In some cases, the second probe may comprise a 5′ phosphorylation (P) modification. In some embodiments the second probe may comprise a 3′ hydroxyl (OH) group. In some embodiments where there are one or more probe (e.g., three probes), a first probe may have a 3′OH end complementary to a target nucleotide sequence, with a 5′P end complementary to an antibody conjugated primer, where a second probe may have a 3′OH end complementary to an antibody conjugated primer, with a 5′P end complementary to a target nucleotide sequence, where a third probe has a 3′OH end and 5′P end complementary to one or more regions within the first and second probe. In some embodiments, the nucleotide of interest is mRNA. In some embodiments, one or more probes may comprise one or more barcodes. In some embodiments of a three probe system, the first probe may comprises an oligonucleotide portion that is complementary to the second probe, a first barcode sequence, and an oligonucleotide portion that is complementary to the third probe, the second probe comprises a portion that recognizes the ribosome and an oligonucleotide portion complementary to the first probe, and the third probe comprises an oligonucleotide portion complementary to the nucleotide of interest, an oligonucleotide portion complementary to the first probe, and a barcode sequence.

In some embodiments, described herein also is a method for assessing RNA translation, the method comprising binding a ribosome with a specific antibody, the specific antibody either bound directly, or indirectly, to one or more antibody-conjugated Nucleotide primer probe(s). The antibody-conjugated Nucleotide primer probe(s) may bind a nucleotide of interest. In some embodiments, the method may comprise ligating the antibody-conjugated Nucleotide primer probe(s) to create a circular oligonucleotide. In some embodiments, the method may comprise amplifying the analyte of interest using the circular oligonucleotide, generating one or more amplicons. In some embodiments, the method may comprise measuring a signal associated with the generation of one or more amplicons. In some embodiments, the one or more antibody-conjugated Nucleotide primer probe(s) comprise one or more reporter barcodes. In some embodiments, the amplicon may be quantified using a fluorescence microscope by in situ sequencing. The number of amplicons and/or fluorescence signal intensities of the amplicons may represent the translation efficiency of the specific mRNA in the specific cells. The amplicon from different target nucleotide species may contain unique barcodes.

The present disclosure also relates to systems that may label nucleic acids bound and/or adjacent to a ribosome, or to other analytes such as DNA/RNA or proteins via proximity ligation. This can be performed in a high-throughput manner in situ to quantitively assess and image relative localization within cells. Briefly, described herein also is a system and methods that may utilize a detection-moiety-conjugated oligonucleotide to recognize a ribosome or adjacent DNA/RNA/protein, and one or more oligo sequences that anneal to RNA/DNA and ligate via RNA- and/or DNA-templated ligation. The resulting ligation product may be a circular DNA template that can be amplified through rolling-circle amplification for subsequent detection. Unique molecular barcodes can be incorporated in the oligonucleotide sequences for oligonucleotide and/or antibody-specific detection.

Additionally, provided herein are novel designs of a primer conjugated to a detection moiety, which may serve as primer and/or ligation template to detect RNA translation and/or RNA interacting analytes in situ. Described herein also is a detection moiety conjugated primer probe system, comprising one or more oligonucleotide probes (e.g. Nucleotide primer probe(s)). At least one 3′OH end and/or at least one 5′P end of the one or more oligonucleotide probes may be complementary to an mRNA. In some cases, upon annealing to a complementary nucleotide (e.g. mRNA or DNA) sequence, the 3′OH and 5′P ends may be located within 0-100 nucleobases of each other. Extension of the ends of the one or more oligonucleotide probes may be performed by reverse transcriptase, which may lead to an adjacent 3′OH and 5′P end annealed to mRNA. The 3′OH and the 5′P ends may be ligated together. The oligonucleotide probe may be circular after ligation. The oligonucleotide probe may comprise one or more unique barcodes. The probe may be amplified after ligation.

In some embodiments, described herein also is a method for assessing RNA translation. The method may comprise binding a ribosome with a specific antibody. The specific antibody may bind directly or indirectly to one or more antibody-conjugated Nucleotide primer probe(s). The antibody-conjugated Nucleotide primer probe(s) may bind a nucleotide of interest (e.g. another probe). In some embodiments, the method may comprise ligating the antibody-conjugated Nucleotide primer probe(s) to create a circular oligonucleotide. In some embodiments, the method may comprise amplifying the nucleotide of interest using the circular oligonucleotide, generating one or more amplicons. In some embodiments, the method may comprise measuring a reporter signal associated with the one or more amplicons. In some embodiments, the one or more antibody-conjugated Nucleotide primer probe(s) may comprise one or more barcodes or derivatives thereof. In some embodiments, one or more amplicons may be quantified using a fluorescence microscope by performing in situ sequencing. Examples of in situ sequencing include sequencing by hybridization, sequencing by ligation, and/or sequencing by synthesis. The number of amplicons or fluorescence signal intensities of the amplicons may represent the translation efficiency of a specific mRNA in cells. The amplicon from different target nucleotide species may have unique barcodes (e.g. each target nucleotide may have a uniquely associated barcode).

In some embodiments, described herein also is a method for assessing RNA translation. The method may comprise binding a ribosome with a specific nucleotide probe. The specific nucleotide probe may be conjugated to a Nucleotide primer probe(s). The Nucleotide primer probe(s) may bind a nucleotide of interest (e.g. an mRNA). In some embodiments, the method may comprise ligating the nucleotide probe-conjugated Nucleotide primer probe(s) to create a circular oligonucleotide. In some embodiments, the method may comprise amplifying the nucleotide of interest using the circular oligonucleotide. One or more amplicons may be generated as a result of amplifying the nucleotide. In some embodiments, the method may further comprise measuring the reporter signal associated with the one or more amplicons.

In some embodiments, described herein also is a method for assessing RNA or DNA and its proximate analytes, such as RNA or DNA modifications and RNA or DNA-binding proteins. The method may comprise binding an analyte with a specific antibody. The specific antibody may be bound directly, or indirectly, to one or more antibody-conjugated Nucleotide primer probe(s). The antibody-conjugated Nucleotide primer probe(s) may bind a nucleotide of interest (e.g. an mRNA). In some embodiments, the method may comprise ligating the antibody-conjugated Nucleotide primer probe(s) to generate a circular oligonucleotide. In some embodiments, the method may comprise amplifying the nucleotide of interest using the circular oligonucleotide to generate one or more amplicons. In some embodiments, the method may comprise measuring a reporter signal associated with the one or more amplicons.

In some embodiments, described herein also is a method for assessing metabolically labeled RNA or RNA with a modified base, such as by ethynyl uridine, 4-thiouridine, bromouridine. The method may comprise binding one or more modified bases. The one or more modified bases may be bound using an antibody that recognizes the one or more modified bases. The one or more modified bases may be bound using chemical conjugation (e.g. using click chemistry). The specific antibody may bind to the one or more modified bases either directly or indirectly. The chemical conjugation used to bind the one or more modified bases may bind the one or more modified bases directly or indirectly. In some embodiments, the method may comprise ligating a probe associated with either the antibody and/or chemical conjugation product to generate a circular oligonucleotide. In some embodiments, the method may comprise amplifying the nucleotide of interest using the circular oligonucleotide to generate one or more amplicons. In some embodiments, the method may comprise measuring a reporter signal associated with the one or more amplicons. In some embodiments, the ligation of the nucleotide primer probe(s) may be templated by endogenous RNA (e.g. an mRNA) by SplintR Ligase. In some embodiments, the ligation of the nucleotide primer probe(s) may be templated by a probe conjugated with unmodified or modified RNA sequences that may serve as a template for SplintR Ligase. In some embodiments, the ligation of the nucleotide primer probe(s) may be templated by nucleotide probe through DNA ligase.

A variety of systems are described herein that relate to detecting the proximity of one or more analytes. Imaging systems used for detecting a signal associated with a label are disclosed herein. Also disclosed herein are computer-implemented methods of performing one or more of the methods disclosed herein.

The imaging systems of the present disclosure may be used for imaging one or more samples as described herein. The imaging systems may comprise a variety of components, including but not limited to a camera, a detector, a filter, a condenser, an objective, or a combination thereof. The objective may be an oil-immersion objective. The objective may be a water-immersion objective. The objective may be an air objective. The imaging system may also comprise a computing system. The computing system may comprise components as described below. The imaging system may also comprise a fluidic system. The fluidic system may comprise one or more pumps, one or more actuators, one or more syringes, one or more robotic units, a tube, a connector, a flow rate sensor, a valve, or a combination thereof. The imaging system may also comprise a sample holder. The sample holder may be configured to hold one or more samples as described herein. The imaging system may also comprise a fluorescent microscope. The fluorescent microscope may be a confocal microscope. The imaging system may comprise a temperature controlling system. The temperature controlling system may comprise a heating element, a cooling element, a fan, a thermometer, or a combination thereof.

Computer implemented methods are disclosed herein for performing one or more of any of the methods described herein using the computer systems described below.

### Computer Systems

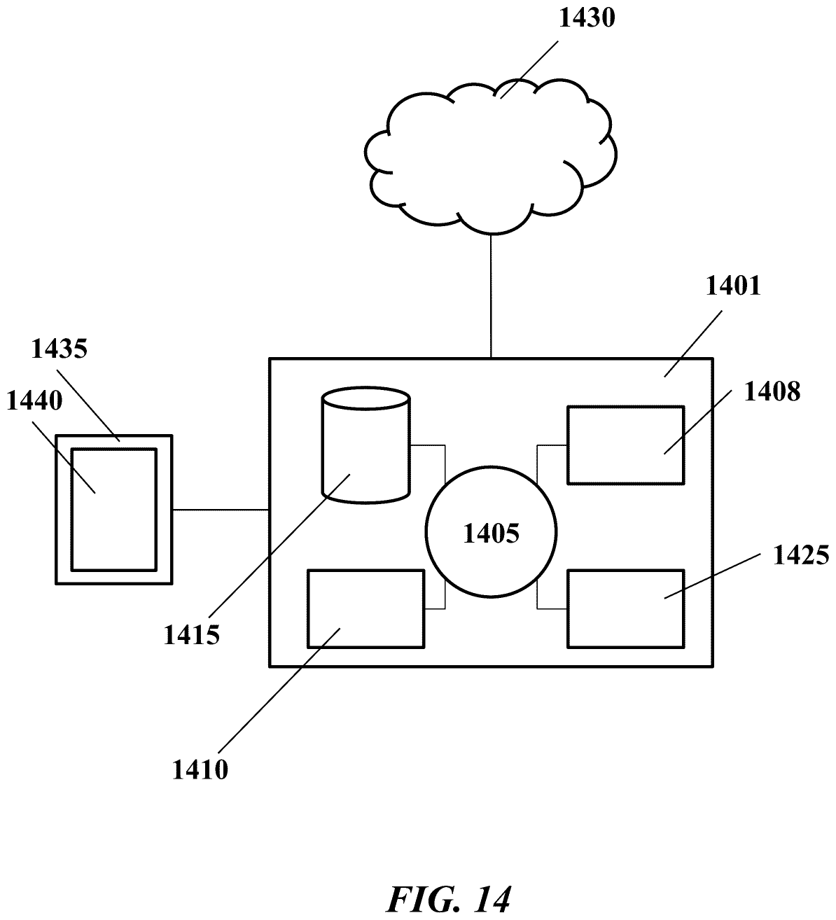

The present disclosure provides computer systems that are programmed to implement methods of the disclosure. FIG. 14 shows a computer system 1401 that is programmed or otherwise configured to control detection of analyte proximity or analysis thereof. The computer system 1401 can regulate various aspects of analyte proximity detection and analysis thereof, such as, for example, temperature control, humidity control, fluid control, camera settings, image analysis, etc. The computer system 1401 can be an electronic device of a user or a computer system that is remotely located with respect to the electronic device. The electronic device can be a mobile electronic device.

The computer system 1401 includes a central processing unit (CPU, also “processor” and “computer processor” herein) 1405, which can be a single core or multi core processor, or a plurality of processors for parallel processing. The computer system 1401 also includes memory or memory location 1410 (e.g., random-access memory, read-only memory, flash memory), electronic storage unit 1415 (e.g., hard disk), communication interface 1408 (e.g., network adapter) for communicating with one or more other systems, and peripheral devices 1425, such as cache, other memory, data storage and/or electronic display adapters. The memory 1410, storage unit 1415, interface 1408 and peripheral devices 1425 are in communication with the CPU 1405 through a communication bus (solid lines), such as a motherboard. The storage unit 1415 can be a data storage unit (or data repository) for storing data. The computer system 1401 can be operatively coupled to a computer network (“network”) 1430 with the aid of the communication interface 1408. The network 1430 can be the Internet, an internet and/or extranet, or an intranet and/or extranet that is in communication with the Internet. The network 1430 in some cases is a telecommunication and/or data network. The network 1430 can include one or more computer servers, which can enable distributed computing, such as cloud computing. The network 1430, in some cases with the aid of the computer system 1401, can implement a peer-to-peer network, which may enable devices coupled to the computer system 1401 to behave as a client or a server.

The CPU 1405 can execute a sequence of machine-readable instructions, which can be embodied in a program or software. The instructions may be stored in a memory location, such as the memory 1410. The instructions can be directed to the CPU 1405, which can subsequently program or otherwise configure the CPU 1405 to implement methods of the present disclosure.

Examples of operations performed by the CPU 1405 can include fetch, decode, execute, and writeback.

The CPU 1405 can be part of a circuit, such as an integrated circuit. One or more other components of the system 1401 can be included in the circuit. In some cases, the circuit is an application specific integrated circuit (ASIC).

The storage unit 1415 can store files, such as drivers, libraries and saved programs. The storage unit 1415 can store user data, e.g., user preferences and user programs. The computer system 1401 in some cases can include one or more additional data storage units that are external to the computer system 1401, such as located on a remote server that is in communication with the computer system 1401 through an intranet or the Internet.

The computer system 1401 can communicate with one or more remote computer systems through the network 1430. For instance, the computer system 1401 can communicate with a remote computer system of a user (e.g., an automated greenhouse). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1401 via the network 1430.

Methods as described herein also can be implemented by way of machine (e.g., computer processor) executable code stored on an electronic storage location of the computer system 1401, such as, for example, on the memory 1410 or electronic storage unit 1415. The machine executable or machine-readable code can be provided in the form of software. During use, the code can be executed by the processor 1405. In some cases, the code can be retrieved from the storage unit 1415 and stored on the memory 1410 for ready access by the processor 1405. In some situations, the electronic storage unit 1415 can be precluded, and machine-executable instructions are stored on memory 1410.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 1401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1401 can include or be in communication with an electronic display 1435 that comprises a user interface (UI) 1440 for providing, for example, operating parameters and conditions, options to control the temperature, humidity level or flow rate, volume dispensed, process progress, etc. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1405. The algorithm can, for example, calculate values, measure variances, analyze image data, analyze tabulated data, measure minimum values, measure maximum values, analyze mass spectrometry data, or calculate flow rates.

### EXAMPLES

### Example 1: In Situ Profiling of DNA-Binding Protein Such as Transcription Factors Binding to DNA

Cells or tissues may be fixed by paraformaldehyde, methanol or a combination thereof. The cells or tissues may be treated with an oligonucleotide-conjugated antibody, for example an anti-STAT3 antibody. A pool of probes, including a first probe comprising a nucleic acid may be added to the cells or tissues. The first probe may comprise a barcode. The first probe comprising the nucleic acid that may be added to the cells or tissues may bind to one or more specific DNA sequences within the cells or tissues. The first probe may bind to the oligonucleotide-conjugated antibody. The first probe may be ligated after hybridizing to the one or more DNA sequences within the cells or tissues using a ligase to form a circular oligonucleotide. The circular oligonucleotide may be amplified using an RCA reaction. The oligonucleotide-conjugated antibody may serve as a primer to initiate the RCA amplification reaction. The RCA reaction may generate one or more amplicons. The one or more amplicons may comprise one or more copies of a reverse complement of the barcode. The reverse complement of the barcode may be detected using sequencing-by-ligation (SBL), for example SEDAL in situ sequencing, sequencing-by-hybridization (SBH), or sequencing-by-synthesis (SBS). The sequencing-by-ligation (SBL), for example SEDAL in situ sequencing, sequencing-by-hybridization (SBH), or sequencing-by-synthesis (SBS) may generate one or more fluorescence spots in the cells or tissues and the number of spots or overall intensity of detected signal of the resulting spots representative of the one or more amplicons may represent the binding status of the specific DNA region of the DNA-binding protein (e.g. STAT3) in a specific cell or tissue. The use of hydrogel-embedding may also be performed to stabilize the position of amplicons.

### Example 2: In Situ Profiling of Histone Modifications

Cells or tissues may be fixed by paraformaldehyde, methanol or a combination thereof. The cells or tissues may be treated with an oligonucleotide-conjugated antibody, for example an anti-histone modification antibody. A pool of probes, including a first probe comprising a nucleic acid may be added to the cells or tissues. The first probe may comprise a barcode. The first probe comprising the nucleic acid that may be added to the cells or tissues may bind to one or more specific DNA sequences within the cells or tissues. The first probe may bind to the oligonucleotide-conjugated antibody. The first probe may be ligated after hybridizing to the one or more DNA sequences within the cells or tissues using a ligase to form a circular oligonucleotide. The circular oligonucleotide may be amplified using an RCA reaction. The oligonucleotide-conjugated antibody may serve as a primer to initiate the RCA amplification reaction. The RCA reaction may generate one or more amplicons. The one or more amplicons may comprise one or more copies of a reverse complement of the barcode. The reverse complement of the barcode may be detected using sequencing-by-ligation (SBL), for example SEDAL in situ sequencing, sequencing-by-hybridization (SBH), or sequencing-by-synthesis (SBS). The sequencing-by-ligation (SBL), for example SEDAL in situ sequencing, sequencing-by-hybridization (SBH), or sequencing-by-synthesis (SBS) may generate one or more fluorescence spots in the cells or tissues and the number of spots or overall intensity of detected signal of the resulting spots representative of the one or more amplicons may represent the binding status of the specific DNA region of the histone modification in a specific cell or tissue. The use of hydrogel-embedding may also be performed to stabilize the position of amplicons.

### Example 3: In Situ Profiling of 5mC DNA Modifications

Cells or tissues may be fixed by paraformaldehyde, methanol or a combination thereof. The cells or tissues may be treated with an oligonucleotide-conjugated antibody, for example an anti-5mC (5-methylcytosine) antibody. A pool of probes, including a first probe comprising a nucleic acid may be added to the cells or tissues. The first probe may comprise a barcode. The first probe comprising the nucleic acid that may be added to the cells or tissues may bind to one or more specific DNA sequences within the cells or tissues. The first probe may bind to the oligonucleotide-conjugated antibody. The first probe may be ligated after hybridizing to the one or more DNA sequences within the cells or tissues using a ligase to form a circular oligonucleotide. The circular oligonucleotide may be amplified using an RCA reaction. The oligonucleotide-conjugated antibody may serve as a primer to initiate the RCA amplification reaction. The RCA reaction may generate one or more amplicons. The one or more amplicons may comprise one or more copies of a reverse complement of the barcode. The reverse complement of the barcode may be detected using sequencing-by-ligation (SBL), for example SEDAL in situ sequencing, sequencing-by-hybridization (SBH), or sequencing-by-synthesis (SBS). The sequencing-by-ligation (SBL), for example SEDAL in situ sequencing, sequencing-by-hybridization (SBH), or sequencing-by-synthesis (SBS) may generate one or more fluorescence spots in the cells or tissues and the number of spots or overall intensity of detected signal of the resulting spots representative of the one or more amplicons may represent the 5mC status of the specific DNA region in a specific cell or tissue. The use of hydrogel-embedding may also be performed to stabilize the position of amplicons.

### Example 4: In Situ Profiling of RNA-Protein Interactions

Cells or tissues may be fixed by paraformaldehyde, methanol or a combination thereof. The cells or tissues may be treated with an oligonucleotide-conjugated antibody, for example an antibody that recognizes polypyrimidine tract binding proteins (PTBPs). A pool of probes, including a first probe comprising a nucleic acid may be added to the cells or tissues. The first probe may comprise a barcode. The first probe comprising the nucleic acid that may be added to the cells or tissues may bind to one or more specific mRNA sequences within the cells or tissues. The first probe may bind to the oligonucleotide-conjugated antibody. The first probe may be ligated after hybridizing to the one or more mRNA sequences within the cells or tissues using a ligase to form a circular oligonucleotide. The circular oligonucleotide may be amplified using an RCA reaction. The oligonucleotide-conjugated antibody may serve as a primer to initiate the RCA amplification reaction. The RCA reaction may generate one or more amplicons. The one or more amplicons may comprise one or more copies of a reverse complement of the barcode. The reverse complement of the barcode may be detected using sequencing-by-ligation (SBL), for example SEDAL in situ sequencing, sequencing-by-hybridization (SBH), or sequencing-by-synthesis (SBS). The sequencing-by-ligation (SBL), for example SEDAL in situ sequencing, sequencing-by-hybridization (SBH), or sequencing-by-synthesis (SBS) may generate one or more fluorescence spots in the cells or tissues and the number of spots or overall intensity of detected signal of the resulting spots representative of the one or more amplicons may represent the protein binding status of the specific mRNA region to PTBPs in a specific cell or tissue. The use of hydrogel-embedding may also be performed to stabilize the position of amplicons.

### Example 5: In Situ Profiling of Metabolically Labeled RNA

Cells or tissues may be treated with 5-ethylyl uridine (5EU) at different time points to metabolically modify RNA. The metabolically modified RNA may be fixed using paraformaldehyde, methanol, or a combination thereof. The cells or tissues may be treated with an oligonucleotide-conjugated to an azide moiety which may be conjugated to 5EU using click chemistry (e.g. Cu-catalyzed click chemistry or copper-free click chemistry). The A pool of probes, including a first probe comprising a nucleic acid may be added to the cells or tissues. The first probe may comprise a barcode. The first probe comprising the nucleic acid that may be added to the cells or tissues may bind to one or more specific mRNA sequences within the cells or tissues. The first probe may be ligated after hybridizing to the one or more mRNA sequences within the cells or tissues using a ligase to form a circular oligonucleotide. The circular oligonucleotide may be amplified using an RCA reaction. The oligonucleotide-conjugated antibody may serve as a primer to initiate the RCA amplification reaction. The RCA reaction may generate one or more amplicons. The one or more amplicons may comprise one or more copies of a reverse complement of the barcode. The reverse complement of the barcode may be detected using sequencing-by-ligation (SBL), for example SEDAL in situ sequencing, sequencing-by-hybridization (SBH), or sequencing-by-synthesis (SBS). The sequencing-by-ligation (SBL), for example SEDAL in situ sequencing, sequencing-by-hybridization (SBH), or sequencing-by-synthesis (SBS) may generate one or more fluorescence spots in the cells or tissues and the number of spots or overall intensity of detected signal of the resulting spots representative of the one or more amplicons may represent the metabolic labeling status of the specific mRNA in a specific cell or tissue. The use of hydrogel-embedding may also be performed to stabilize the position of amplicons.

### Example 6: In Situ Profiling of m6A RNA Modifications

Cells or tissues may be fixed by paraformaldehyde, methanol or a combination thereof. The cells or tissues may be treated with an oligonucleotide-conjugated antibody, for example an anti-N⁶-Methyladenosine (m6A) antibody. A pool of probes, including a first probe comprising a nucleic acid may be added to the cells or tissues. The first probe may comprise a barcode. The first probe comprising the nucleic acid that may be added to the cells or tissues may bind to one or more specific mRNA sequences within the cells or tissues. The first probe may bind to the oligonucleotide-conjugated antibody. The first probe may be ligated after hybridizing to the one or more mRNA sequences within the cells or tissues using a ligase to form a circular oligonucleotide. The circular oligonucleotide may be amplified using an RCA reaction. The oligonucleotide-conjugated antibody may serve as a primer to initiate the RCA amplification reaction. The RCA reaction may generate one or more amplicons. The one or more amplicons may comprise one or more copies of a reverse complement of the barcode. The reverse complement of the barcode may be detected using sequencing-by-ligation (SBL), for example SEDAL in situ sequencing, sequencing-by-hybridization (SBH), or sequencing-by-synthesis (SBS). The sequencing-by-ligation (SBL), for example SEDAL in situ sequencing, sequencing-by-hybridization (SBH), or sequencing-by-synthesis (SBS) may generate one or more fluorescence spots in the cells or tissues and the number of spots or overall intensity of detected signal of the resulting spots representative of the one or more amplicons may represent the m6A status of the specific DNA region in a specific cell or tissue. The use of hydrogel-embedding may also be performed to stabilize the position of amplicons.

### Example 7: In Situ Profiling of RNA Translation Status

Cells or tissues may be fixed by paraformaldehyde, methanol or a combination thereof. The cells or tissues may be treated with an oligonucleotide-conjugated antibody, for example an antibody recognizing a ribosomal protein, or an antisense probe recognizing a ribosomal RNA. A pool of probes, including a first probe comprising a nucleic acid may be added to the cells or tissues. The first probe may comprise a barcode. The first probe comprising the nucleic acid that may be added to the cells or tissues may bind to one or more specific mRNA sequences within the cells or tissues. The first probe may bind to the oligonucleotide-conjugated antibody or the antisense probe recognizing a ribosomal RNA. The first probe may be ligated after hybridizing to the one or more mRNA sequences within the cells or tissues using a ligase to form a circular oligonucleotide. The circular oligonucleotide may be amplified using an RCA reaction. The oligonucleotide-conjugated antibody may serve as a primer to initiate the RCA amplification reaction. The RCA reaction may generate one or more amplicons. The one or more amplicons may comprise one or more copies of a reverse complement of the barcode. The reverse complement of the barcode may be detected using sequencing-by-ligation (SBL), for example SEDAL in situ sequencing, sequencing-by-hybridization (SBH), or sequencing-by-synthesis (SBS). The sequencing-by-ligation (SBL), for example SEDAL in situ sequencing, sequencing-by-hybridization (SBH), or sequencing-by-synthesis (SBS) may generate one or more fluorescence spots in the cells or tissues and the number of spots or overall intensity of detected signal of the resulting spots representative of the one or more amplicons may represent the translation status of the specific DNA region in a specific cell or tissue. The use of hydrogel-embedding may also be performed to stabilize the position of amplicons.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method of detecting analytes in a sample, said method comprising:
(a) providing a first probe and a second probe,
wherein said first probe comprises:
(i) a first binding site configured to couple to a first analyte at a first portion;
(ii) a second binding site configured to couple to said first analyte at a second portion, wherein said first portion is adjacent to said second portion;
(iii) a third binding site configured to couple to said second probe;
(iv) a barcode;
(v) a first end; and
(vi) a second end;
wherein said second probe comprises:
(vii) a fourth binding site configured to couple to said first probe; and
(viii) a fifth binding site configured to couple to said second analyte;
(b) contacting a sample comprising a plurality of analytes comprising said first analyte and said second analyte with said first probe and said second probe, such that:
(i) said first probe is coupled to said first analyte;
(ii) said second probe is coupled to said second analyte; and
(iii) said first probe is coupled to said second probe; and
(c) detecting said barcode or a derivative thereof using a plurality of detection probes, thereby determining a proximity of said first analyte to said second analyte.

2. The method of claim 1, wherein said sample is a tissue sample.

3. The method of claim 1, wherein said first analyte comprises a nucleic acid.

4. The method of claim 3, wherein said nucleic acid comprises a ribonucleic acid.

5. The method of claim 3, wherein said nucleic acid comprises a single nucleotide polymorphism.

6. The method of claim 5, wherein said first probe recognizes said single nucleotide polymorphism

7. The method of claim 1, wherein said first analyte comprises a polypeptide.

8. The method of claim 1, wherein said second analyte comprises a polypeptide.

9. The method of claim 1, wherein said second analyte comprises a nucleic acid.

10. The method of claim 1, wherein said first probe comprises a nucleic acid.

11. The method of claim 10, wherein said nucleic acid comprises one or more modifications.

12. The method of claim 1, wherein said second probe comprises a nucleic acid.

13. The method of claim 12, wherein said nucleic acid comprises one or more modifications.

14. The method of claim 1, wherein said second probe comprises a polypeptide.

15. The method of claim 14, wherein said polypeptide comprises an antibody or antibody fragment.

16. The method of claim 1, wherein said second probe recognizes a polypeptide.

17. The method of claim 16, wherein said polypeptide is a protein.

18. The method of claim 17, wherein said protein is a transcription factor.

19. The method of claim 17, wherein said protein is a ribosomal protein.

20. The method of claim 1, further comprising ligating said first end and said second end to form a circular oligonucleotide.

21. The method of claim 20, wherein said ligating comprises contacting said sample with a ligase.

22. The method of claim 20, further comprising amplifying said circular oligonucleotide to generate an amplicon.

23. The method of claim 22, wherein said amplifying comprises performing a rolling circle amplification reaction.

24. The method of claim 1, wherein said barcode comprises a nucleic acid.

25. The method of claim 1, wherein (c) comprises hybridizing a detection probe and an anchor probe of said plurality of detection probes to said amplicon.

26. The method of claim 25, wherein said detection probe comprises a label.

27. The method of claim 26, wherein said label comprises a fluorescent molecule.

28. The method of claim 1, wherein said amplicon comprises a derivative of said barcode, and said derivative of said barcode comprises a reverse complement of said barcode.

29. The method of claim 1, wherein (c) comprises imaging said sample.

30. The method of claim 1, wherein said sample is embedded in a hydrogel.
